(19) United States
(12) Patent Application Publication
(10) Pub. No.: US 2024/0109939 A1
(43) Pub. Date: Apr. 4, 2024

(54) POOLS OF MICROBIAL PROTEIN FRAGMENTS

(71) Applicant: Oxford Immunotec Limited, Abingdon (GB)

(72) Inventor: Daniel Cochrane, Oxford (GB)

(73) Assignee: Oxford Immunotec Limited, Abingdon (GB)

(21) Appl. No.: 18/272,779

(22) PCT Filed: Jan. 26, 2022
(86) PCT No.: PCT/GB2022/050199

§ 371 Date: Jul. 17, 2023

(30) Foreign Application Priority Data
Jan. 27, 2021 (GB) 2101078.0

(51) Int. Cl. C07K 14/005; G01N 33/50

(52) U.S. Cl. CPC C07K 14/005; G01N 33/5047; G01N 2333/165

(57) ABSTRACT

The disclosure concerns a method for producing a pool of fragments derived from a microbial protein. The disclosure also concerns a pool of fragments derived from a microbial protein, and a method for determining the presence or absence of immune cells targeting a microbe.

### FIELD OF THE DISCLOSURE

The disclosure concerns a method for producing a pool of fragments derived from a microbial protein. The disclosure also concerns a pool of fragments derived from a microbial protein, and a method for determining the presence or absence of immune cells targeting a microbe.

### BACKGROUND

Microbes, such as viruses, bacteria, fungi and protozoa, are a common cause of disease in humans and animals. Some microbial infections may cause mild disease symptoms, and others severe disease or even death.

Immune protection to microbial disease may be elicited in both humans and animals. One mechanism of immune protection involves antibody generation. Another mechanism involves the generation and priming of T cells responsive to the microbe. In either case, an initial encounter with a first microbe may elicit immune protection against a further encounter with that microbe. An initial encounter with a first microbe may also elicit immune protection against a second microbe that is different from the first microbe. In other words, the immune protection elicited in response to the first microbe may be cross-protective against infection with a second microbe.

Cross-protective immunity may exist between related microbes, such as microbes belonging to the same family. For example, cross-protective immunity is thought to exist between different human coronaviruses. Animal data and limited human epidemiological data indicate that T cell mediated immune protection to severe acute respiratory syndrome coronavirus 2 (SARS-CoV-2) mediated disease can be elicited. SARS-CoV-2 responsive T cells may be generated in individuals symptomatically or asymptomatically infected with SARS-CoV-2. Additionally, SARS-CoV-2 responsive T cells have been described in a proportion of the SARS-CoV-2 naive population. These cells are likely primed by infection with the endemic common cold Coronaviridae (CCCs). That is, an initial encounter with an endemic common cold coronavirus may provide cross-protection against a subsequent encounter with SARS-CoV-2.

Microbe-specific immune responses may be characterised using a number of methods known in the art. For example, cell mediated immunity to a microbe may be characterised by contacting a sample containing immune cells with one or more antigens from the microbe, and detecting the presence, absence or characteristics of an immune response to the one or more antigens. Each antigen may, for example, comprise one or more peptides or proteins from the microbe. While cross-protection may be beneficial to the individual encountering the microbe(s), it can complicate the characterisation of microbe-specific immune responses such as cell mediated immune responses. This can pose challenges to research into, and diagnosis of, microbial diseases. There is therefore a need for a toolkit that enables cell mediated immune responses elicited by a microbe of interest to be distinguished from cross-reactive cell mediated immune responses elicited by a different (e.g. related) microbe.

### SUMMARY

Some assays for cell mediated immunity to a microbe of interest detect the presence, absence or characteristics of an immune response of immune cells in a sample to a pool of fragments from a protein from the microbe (i.e. a microbial protein). The pool of fragments is essentially used as the test antigen in the assay. Providing the antigen as a pool of fragments may help to account for variations in immune repertoire between individuals, because the number of potential epitopes with which the immune cells are contacted is maximised. In certain cases, the fragments comprised in the pool form a protein fragment library that encompasses some or all of the sequence of the microbial protein. The present inventors have developed a method for producing such a pool of fragments, which pool is optimised for use in an assay for cell mediated immunity.

In more detail, the present inventors have developed a method of producing a pool of fragments that is optimised for assaying (I) cell mediated immunity that is cross-reactive for the microbe of interest, or (II) cell mediated immunity that is specific for the microbe of interest. This allows the nature of cell mediated immunity for a microbe of interest to be better characterised. This may be beneficial in a research or diagnostic context, where it is desirable to distinguish true microbe-specific immunity from immunity that is elicited from a different but related microbe. For example, it may be advantageous to distinguish cell mediated immunity elicited by exposure to the emerging pathogen SARS-CoV-2 from that elicited by exposure to endemic common cold Coronaviridae, as this may improve the specificity of diagnosis and disease surveillance. The same may apply to other emerging and endemic pathogens.

Accordingly, the disclosure provides a method for producing a pool of fragments derived from a microbial protein, comprising: (a) identifying fragments of the microbial protein that are comprised in a protein fragment library encompassing at least 80% of the sequence of the microbial protein; (b) determining for each fragment identified in step (a) whether or not a homolog exists, wherein the homolog is an amino acid sequence that has at least 60% sequence identity to the fragment and is expressed by one or more microbes in the same family as the microbe from which the microbial protein is derived; and (c) preparing a pool of fragments in which: (i) each fragment is a fragment identified in step (a) for which step (b) determines the existence of a homolog; or (ii) each fragment is a fragment identified in step (a) for which step (b) does not determine the existence of a homolog, and the fragments form a protein fragment library encompassing at least 80% of the sequence of the microbial protein.

The invention also provides:
a pool of fragments derived from a microbial protein, wherein: (I) each fragment is comprised in a protein fragment library encompassing at least 80% of the sequence of the microbial protein, and has a homolog that is an amino acid sequence that has at least 60% sequence identity to the fragment and is expressed by one or more microbes in the same family as the microbe from which the microbial protein is derived; or (II) the fragments form a protein fragment library encompassing at least 80% of the sequence of the microbial protein, and each fragment does not have a homolog that is an amino acid sequence that has at least 60% sequence identity to the fragment and is expressed by one or more microbes in the same family as the microbe from which the microbial protein is derived;
a consolidated pool of fragments which comprises two or more pools of the invention, wherein each of the two or more pools comprises fragments derived from a different microbial protein, optionally wherein the microbial protein is selected from SARS-CoV-2 S1 spike domain, SARS-CoV-2 S2 spike domain, SARS-CoV-2 nucleocapsid protein, SARS-CoV-2 membrane protein, and SARS-CoV-2 envelope protein; and
a method for determining the presence or absence of immune cells targeting a microbe, the method comprising contacting a sample comprising immune cells with one or more pools of the invention, and detecting in vitro the presence or absence of an immune response to the pool.

### BRIEF DESCRIPTION OF THE FIGURES

FIG. 1: graphical representation of P1-4, P13 and P7-10.

### DETAILED DESCRIPTION

It is to be understood that different applications of the disclosed methods and products may be tailored to the specific needs in the art. It is also to be understood that the terminology used herein is for the purpose of describing particular embodiments of the disclosure only, and is not intended to be limiting.

In addition, as used in this specification and the appended claims, the singular forms “a”, “an”, and “the” include plural referents unless the content clearly dictates otherwise. Thus, for example, reference to “a cell” includes “cells”, reference to “an image” includes two or more such images, reference to “an antigen” includes two or more such antigens, and the like.

All publications, patents and patent applications cited herein, whether supra or infra, are hereby incorporated by reference in their entirety.

### Method for Producing a Pool of Fragments

Disclosed herein is a method for producing a pool of fragments derived from a microbial protein, comprising: (a) identifying fragments of the microbial protein that are comprised in a protein fragment library encompassing at least 80% of the sequence of the microbial protein; (b) determining for each fragment identified in step (a) whether or not a homolog exists, wherein the homolog is an amino acid sequence that has at least 60% sequence identity to the fragment and is expressed by one or more microbes in the same family as the microbe from which the microbial protein is derived; and (c) preparing a pool of fragments in which: (i) each fragment is a fragment identified in step (a) for which step (b) determines the existence of a homolog; or (ii) each fragment is a fragment identified in step (a) for which step (b) does not determine the existence of a homolog, and the fragments form a protein fragment library encompassing at least 80% of the sequence of the microbial protein.

The features and advantages of the method are described in detail below.

### Fragments and Fragment Pools

The method produces a pool of fragments derived from a microbial protein. The pool of fragments is a pool in which (i) each fragment is a fragment identified in step (a) for which step (b) determines the existence of a homolog; or (ii) each fragment is a fragment identified in step (a) for which step (b) does not determine the existence of a homolog, and the fragments form a protein fragment library encompassing at least 80% of the sequence of the microbial protein.

In more detail, each fragment comprised in the pool of fragments (i) is a fragment that is identified as being comprised in a protein fragment library encompassing at least 80% of the sequence of the microbial protein. The fragments comprised in the pool (i) need not themselves form such a protein fragment library. Rather, each fragment comprised in the pool of fragments (i) is a fragment that is notionally comprised in a protein fragment library that encompasses at least 80% of the sequence of the microbial protein. In other words, each fragment comprised in the pool of fragments (i) is a fragment that is or would be found in a protein fragment library that encompasses at least 80% of the sequence of the microbial protein. Each fragment comprised in the pool of fragments (i) is also a fragment that has a homolog which is an amino acid sequence that has at least 60% sequence identity to the fragment and is expressed by one or more microbes in the same family as the microbe from which the microbial protein is derived. Homologs are described in detail below.

Accordingly, the pool of fragments (i) essentially comprises fragments that are not unique to the microbe from which the microbial protein is derived. The pool of fragments (i) may thus comprise fragments that may be recognised by a cross-reactive immune response. That is, the pool of fragments (i) may comprise fragments that are recognised by (e.g. bind to antigen receptors on and/or trigger a response by) immune cells that are generated by contact with a microbe other that the microbe from which the microbial protein is derived.

Each fragment comprised in the pool of fragments (ii) is a fragment that is identified as being comprised in a protein fragment library encompassing at least 80% of the sequence of the microbial protein. In other words, each fragment comprised in the pool of fragments (ii) is a fragment that is notionally comprised in a protein fragment library that encompasses at least 80% of the sequence of the microbial protein. That is, each fragment comprised in the pool of fragments (ii) is a fragment that is or would be found in a protein fragment library that encompasses at least 80% of the sequence of the microbial protein. In addition, the fragments comprised in the pool (ii) themselves form protein fragment library encompassing at least 80% of the sequence of the microbial protein. Furthermore, each fragment comprised in the pool of fragments (ii) is a fragment that does not have a homolog which is an amino acid sequence that has at least 60% sequence identity to the fragment and is expressed by one or more microbes in the same family as the microbe from which the microbial protein is derived. Homologs and protein fragment libraries are described in detail below.

Accordingly, the pool of fragments (ii) essentially comprises fragments that are unique to the microbe from which the microbial protein is derived. In other words, the pool of fragments (ii) essentially comprises only fragments that do not have a homolog in another microbe belonging to the same family as the microbe from which the microbial protein is derived. Thus, the pool of fragments (ii) may exclude fragments that may be recognised by a cross-reactive immune response. That is, the pool of fragments (ii) may exclude fragments that are recognised by (e.g. bind to antigen receptors on and/or trigger a response by) immune cells generated by contact with a microbe other that the microbe from which the microbial protein is derived.

In either case, a fragment derived from a microbial protein may be an amino acid sequence, or a peptide. For example, a fragment derived from a microbial protein may be a sequence comprising five or more amino acids that is derived by truncation at the N-terminus and/or C-terminus of the sequence of the microbial protein (“the parent sequence”). For instance, the fragment may comprise about 5 or more, about 6 or more, about 7 or more, about 8 or more, about 9 or more, about 10 or more, about 11 or more, about 12 or more, about 13 or more, about 14 or more, about 15 or more, about 16 or more, about 17 or more, about 18 or more, about 19 or more, about 20 or more, about 21 or more, about 22 or more, about 23 or more, about 24 or more, about 25 or more, about 26 or more, about 27 or more, about 28 or more, about 29 or more or about 30 or more amino acids. The fragment may be from about 5 to about 30, from about 6 to about 29, from about 7 to about 28, from about 8 to about 27, from about 9 to about 26, from about 10 to about 25, from about 11 to about 24, from about 12 to about 23, from about 13 to about 22, from about 14 to about 21, from about 15 to about 20, from about 16 to about 19, or from about 17 to about 18 amino acids in length. The fragment may, for example, be from about 9 to about 20, about 10 to about 19, about 11 to about 18, about 12 to about 17, about 13 to about 16, or about 15 amino acids in length. Preferably, the fragment is about 15 amino acids in length.

The term “fragment” includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol. 159, 3230-3237. This approach involves making pseudopeptides containing changes involving the backbone, and not the orientation of side chains. Meziere et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the fragment may conveniently be blocked at its N- or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. One or more additional amino acid residues may also be added at the N-terminus and/or C-terminus of the fragment, for example to increase the stability of the fragment. Other examples of modifications include glycosylation and phosphorylation. Another potential modification is that hydrogens on the side chain amines of R or K may be replaced with methylene groups (—NH₂→—NH(Me) or —N(Me)₂).

Fragments of the microbial protein may include variants of fragments that increase or decrease the fragments' longevity in vitro or in vivo. Examples of variants capable of increasing the longevity of fragments according to the invention include peptoid analogues of the fragments, D-amino acid derivatives of the fragments, and peptide-peptoid hybrids. The fragment may also comprise D-amino acid forms of the fragment. The preparation of polypeptides using D-amino acids rather than L-amino acids greatly decreases any unwanted breakdown of such an agent by normal metabolic processes, decreasing the amounts of agent which needs to be administered, along with the frequency of its administration. D-amino acid forms of the parent protein may also be used.

The fragments may be derived from splice variants of the parent protein encoded by mRNA generated by alternative splicing of the primary transcripts encoding the parent protein chains. The fragments may also be derived from amino acid mutants, glycosylation variants and other covalent derivatives of the parent proteins which retain at least an MHC-binding or antibody-binding property of the parent protein. Exemplary derivatives include molecules wherein the fragments of the invention are covalently modified by substitution, chemical, enzymatic, or other appropriate means with a moiety other than a naturally occurring amino acid.

A pool of fragments derived from a microbial protein comprises two or more fragments of the microbial protein. Fragments are described above. A pool may, for example, comprise three or more, four or more, five or more, six or more, seven or more, eight or more, nine of more, 10 or more, 15 or more, 20 or more, 25 or more, 50 or more, 75 or more, 100 or more, 200 or more, or 250 or more, fragments of the microbial protein.

The fragments comprised in a pool may form a protein fragment library. A protein fragment library comprises a plurality of fragments derived from a parent protein (in the present disclosure, the microbial protein), that together encompass at least 10%, such as at least 20%, at least 30%, at least 40%, at least 50%, at least 60%, at least 70%, at least 75%, at least 80%, at least 85%, at least 90%, at least 95%, at least 98%, at least 99%, or 100%, of the sequence of the parent protein. In the pool of fragments (ii), the fragments form a protein fragment library encompassing at least 80% of the sequence of the parent protein. For example, the fragments may form a protein fragment library encompassing the entire sequence of the parent protein. In a protein fragment library in which the fragments together encompass at least 10% (such as at least 20%, at least 30%, at least 40%, at least 50%, at least 60%, at least 70%, at least 75%, at least 80%, at least 85%, at least 90%, at least 95%, at least 98%, at least 99%, or 100%) of the sequence of the parent protein, the fragments are diverse enough that the pool contains epitopes capable of binding to many different MHC alleles. This allows the pool to be used in assays for cell mediated immunity across the global population, despite variation in MHC alleles between subjects.

The protein fragment library may comprise fragments that are capable of stimulating CD4+ and/or CD8+ T cells. The protein fragment library may comprise fragments that are capable of stimulating both CD8+ T cells and CD4+ T cells. It is known in the art that the optimal fragment size for stimulation is different for CD4+ and CD8+ T-cells. Fragments consisting of about 9 amino acids (9mers) typically stimulate CD8+ T-cells only, and fragments consisting of about 20 amino acids (20mers) typically stimulate CD4+ T-cells only. Broadly speaking, this is because CD8+ T-cells tend to recognise their antigen based on its sequence, whereas CD4+ T-cells tend to recognise their antigen based on its higher-level structure. However, fragments consisting of about 15 amino acids (15mers) may stimulate both CD4+ and CD8+ T cells. The protein fragment library preferably comprises fragments that are about 15 amino acids, such as about 12 amino acids, about 13 amino acids, about 14 amino acids, about 16 amino acids, about 17 amino acids or about 18 amino acids in length.

All of the fragments in the protein fragment library may be the same length. Alternatively, the protein fragment library may comprise fragments of different lengths. Fragment lengths are discussed above.

The protein fragment library may comprise fragments whose sequences overlap. The sequences may overlap by one or more, such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, or 20 or more, amino acids. Preferably, the sequences overlap by 9 or more amino acids, such as 10 or more, 11 or more or 12 or more amino acids, as this maximises the number of fragments that comprise 9mers capable of stimulating CD8+ T cells. More preferably, the sequences overlap by 11 amino acids. All of the overlapping fragments in the protein fragment library may overlap by the same number of amino acids. Alternatively, the protein fragment library may comprise fragments whose sequences overlap by different numbers of amino acids.

The protein fragment library may, for example, comprise fragments of 12 to 18 (such as 12 to 15, 15 to 18, 13 to 17, or 14 to 16) amino acids in length that overlap by 9 to 12 (such as 9 to 11 or 10 to 12) amino acids. For instance, the protein fragment library may comprise fragments of (a) 14 amino acids in length that overlap by 9, 10, or 11 amino acids, (b) 15 amino acids in length that overlap by 9, 10, or 11 amino acids, or (c) 16 amino acids in length that overlap by 9, 10, or 11 amino acids. The protein fragment library preferably comprises fragments of 15 amino acids in length that overlap by 11 amino acids.

### Microbial Protein

The fragments comprised in the pool produced by the method of the disclosure are derived from a microbial protein. A microbial protein is a protein that is expressed by a microbe.

Microbes are well-known in the art and include viruses, bacteria, fungi and protozoa. Accordingly, the microbial protein may be expressed by a virus. In this case, the microbial protein is a viral protein. The microbial protein may be expressed by a bacterium. In this case, the microbial protein is a bacterial protein. The microbial protein may be expressed by a fungus. In this case, the microbial protein is a fungal protein. The microbial protein may be expressed by a protozoa. In this case, the microbial protein is a protozoal protein.

The microbe from which the microbial protein is derived may be a pathogenic microbe. That is, the microbe may be capable of causing disease. The microbe from which the microbial protein is derived may be a non-pathogenic microbe. That is, the microbe may be one that does not typically cause disease. For instance, the microbe may be a commensal microbe.

In one aspect of the disclosure, the microbe from which the microbial protein is derived is an emerging pathogen. An emerging pathogen may be defined as the causative microbe of an infectious disease whose incidence is increasing following its appearance in a new host population or whose incidence is increasing in an existing population as a result of long-term changes in its underlying epidemiology. Typically, an emerging pathogen is a virus, a bacterium or a protozoa. Emerging diseases have, in recent years, included respiratory, central nervous system, and enteric infections, viral hemorrhagic fevers, hepatitides, systemic bacterial infections, and human retroviral and novel herpes viral infections. Emerging viruses have included HIV, hepatitis C virus, ebola virus, nipah virus, lassa virus, and West Nile virus, for example. Emerging bacteria have included E. coli O157, Vibrio cholerae O139, Clostridium difficile, Legionella pneumophila, and Campylobacter jejuni/coli, for example. Emerging pathogens of particular note include novel human coronavirues such as SARS-CoV-2, which is responsible for an ongoing global pandemic.

In a preferred aspect of the disclosure, the microbe is a virus. Preferably, the virus is a virus of the realm Riboviria. Preferably, the virus is a virus of the kingdom Orthornavirae. Preferably, the virus is a virus of the phylum Pisuviricota. Preferably, the virus is a virus of the class Pisoniviricetes. Preferably, the virus is a virus of the order Nidovirales. Preferably, the virus is a virus of the family Coronaviridae. Thus, the microbe is preferably a coronavirus. The coronavirus may, for example, be SARS-CoV-2.

The protein may be expressed on the surface of the microbe. That is, the microbial protein may be a surface microbial protein. The microbial protein may be expressed internally within the microbe. That is, the microbial protein may be an internal microbial protein. If the microbe is a bacterium, fungus, or protozoa, the internal protein may be an intracellular protein. If the microbe is a virus, the internal protein may be an intraviral protein.

The protein may be any type of protein. For example, the protein may be a structural protein. The protein may, for example, be an enzyme. The protein may, for example, be a receptor. The protein may, for example, be a transport molecule. The protein may, for example, be a transcription factor.

The protein may be an antigenic protein. An antigenic protein is a protein that may function as an antigen. In other words, an antigenic protein is a protein that comprises a peptide that is capable of binding to an immune receptor. For instance, an antigenic protein may comprise a peptide that is capable of binding to an antibody. An antigenic protein may comprise a peptide that is capable of binding to an B cell receptor. An antigenic protein may comprise a peptide that is capable of binding to a T cell receptor, such as an alpha-beta T cell receptor or a gamma-delta T cell receptor. In the present disclosure, the antigenic protein is preferably capable of binding to a T cell receptor.

As set out above, the microbe from which the microbial protein is derived is preferably a coronavirus, such as SARS-CoV-2. Accordingly, the microbial protein is preferably a coronavirus protein. The coronavirus protein may, for example, be a SARS-CoV-2 protein. Preferably, the SARS-CoV-2 protein is a structural protein. SARS-CoV-2 structural proteins include SARS-CoV-2 S1 spike glycoprotein (which comprises SARS-CoV-2 S1 spike domain (S1) and SARS-CoV-2 S2 spike domain (S2)), SARS-CoV-2 nucleocapsid protein (N), SARS-CoV-2 membrane protein (M), and SARS-CoV-2 envelope protein (E).

Step (a)—Identifying Fragments Comprised in a Protein Fragment Library

Step (a) of the method comprises identifying fragments of the microbial protein that are comprised in a protein fragment library encompassing at least 80% of the sequence of the microbial protein. The protein fragment library comprises a plurality of fragments derived from the microbial protein, that together encompass at least 80% (such as at least 85%, at least 90%, at least 95%, at least 98%, at least 99%, or 100%) of the sequence of the microbial protein.

The protein fragment library may comprise fragments that are capable of stimulating CD4+ and/or CD8+ T cells. The protein fragment library may comprise fragments that are capable of stimulating both CD8+ T cells and CD4+ T cells. As explained above, it is known in the art that the optimal fragment size for stimulation is different for CD4+ and CD8+ T-cells. Fragments consisting of about 9 amino acids (9mers) typically stimulate CD8+ T-cells only, and fragments consisting of about 20 amino acids (20mers) typically stimulate CD4+ T-cells only. Fragments consisting of about 15 amino acids (15mers) may stimulate both CD4+ and CD8+ T cells. The protein fragment library may therefore comprise fragments that are from about 9 to about 20 (such as about 10 to about 19, about 11 to about 18, about 12 to about 17, about 13 to about 16, or about 15) amino acids in length. The protein fragment library preferably comprises fragments that are about 15 amino acids, such as about 12 amino acids, about 13 amino acids, about 14 amino acids, about 16 amino acids, about 17 amino acids or about 18 amino acids in length. All of the fragments in the protein fragment library may be the same length. Alternatively, the protein fragment library may comprise fragments of different lengths. Fragment lengths are discussed above.

The protein fragment library may comprise fragments whose sequences overlap. The sequences may overlap by one or more, such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, or 20 or more, amino acids. Preferably, the sequences overlap by 9 or more amino acids, such as 10 or more, 11 or more or 12 or more amino. More preferably, the sequences overlap by 11 amino acids. All of the overlapping fragments in the protein fragment library may overlap by the same number of amino acids. Alternatively, the protein fragment library may comprise fragments whose sequences overlap by different numbers of amino acids.

The protein fragment library may, for example, comprise fragments of 12 to 18 (such as 12 to 15, 15 to 18, 13 to 17, or 14 to 16) amino acids in length that overlap by 9 to 12 (such as 9 to 11 or 10 to 12) amino acids. For instance, the protein fragment library may comprise fragments of (a) 14 amino acids in length that overlap by 9, 10, or 11 amino acids, (b) 15 amino acids in length that overlap by 9, 10, or 11 amino acids, or (c) 16 amino acids in length that overlap by 9, 10, or 11 amino acids. The protein fragment library preferably comprises fragments of 15 amino acids in length that overlap by 11 amino acids.

Methods for identifying fragments of the microbial protein that are comprised in the protein fragment library are known in the art. For example, the amino acid sequence of the microbial protein may be processed to an algorithm that returns a list of fragments comprised in a protein fragment library that encompasses an inputted percentage of the amino acid sequence of the microbial protein, and comprises fragments of an inputted length and overlap. A similar exercise could be performed manually.

Step (b)—Determining the Existence of a Homolog

Step (b) of the method comprises determining for each fragment identified in step (a) whether or not a homolog exists. In this context, a homolog is defined as an amino acid sequence that has at least 60% sequence identity to the fragment and is expressed by one or more microbes in the same family as the microbe from which the microbial protein is derived. As set out above, the pool of fragments (i) produced in step (c) contains only fragments having such a homolog. The pool of fragments (ii) produced in step (c) excludes fragments having such a homolog.

The homolog may, for example, have at least 60%, at least 65%, at least 70%, at least 75%, at least 80%, at least 85%, at least 90%, at least 95%, at least 96%, at least 97%, at least 98%, at least 99% or 100% sequence identity to the respective fragment. For the purpose of this disclosure, in order to determine the percent identity of two sequences (such as two amino acid sequences), the sequences are aligned for optimal comparison purposes (e.g., gaps can be introduced in a first sequence for optimal alignment with a second sequence). The nucleotide residues at nucleotide positions are then compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 60% identical to SEQ ID NO: X (an example of a reference sequence), the skilled person would carry out an alignment over the length of SEQ ID NO: X, and identify how many positions in the test sequence were identical to those of SEQ ID NO: X. If at least 60% of the positions are identical, the test sequence is at least 60% identical to SEQ ID NO: X. If the sequence is shorter than SEQ ID NO: X, the gaps or missing positions should be considered to be non-identical positions. SEQ ID NO: X may be taken to represent a fragment identified in step (a) of the method. The “test sequence” may be taken to represent a potential homolog.

The skilled person is aware of different computer programs that are available to determine the homology or identity between two sequences. For instance, a comparison of sequences and determination of percent identity between two sequences can be accomplished using a mathematical algorithm.

As set out above, the fragments identified in step (a) of the method are preferably 15 amino acids in length. An amino acid sequence having at least 60% sequence identity to a 15 amino acid fragment may comprise 9 or more (such as 10 or more, 11 or more, 12 or more, 13 or more, 14 or more, or 15) positions that are identical to those in the 15 amino acid fragment. For example, an amino acid sequence having at least 60% sequence identity to a 15 amino acid fragment may comprise 9 to 15 (such as 10 to 14, or 12 to 13) positions that are identical to those in the 15 amino acid fragment.

An amino acid sequence having at least 60% sequence identity to a 15 amino acid fragment may comprise one or more amino acid substitutions with respect to the 15 amino acid fragment. For example, the amino acid sequence may comprise one, two, three, four, five or six amino acid substitutions with respect to the 15 amino acid fragment, providing that the amino acid sequence comprises 9 or more positions that are identical to those in the 15 amino acid fragment. An amino acid sequence having at least 60% sequence identity to a 15 amino acid fragment may comprise one or more amino acid deletions with respect to the 15 amino acid fragment. For example, the amino acid sequence may comprise one, two, three, four, five or six amino acid deletions with respect to the 15 amino acid fragment, providing that the amino acid sequence comprises 9 or more positions that are identical to those in the 15 amino acid fragment. An amino acid sequence having at least 60% sequence identity to a 15 amino acid fragment may comprise any number and combination of amino acid substitutions and amino acid deletions, providing that the amino acid sequence comprises 9 or more positions that are identical to those in the 15 amino acid fragment.

The homolog is expressed by one or more microbes in the same family as the microbe from which the microbial protein is derived. For example, the homolog may be expressed by two or more, three or more, four or more, five or more, six or more, seven or more, eight or more, nine or more, or 10 or more microbes in the same family as the microbe from which the microbial protein is derived. In this context, the term “family” refers to a taxonomic family. By way of non-limiting example, the microbial protein may be expressed by a first virus in the Coroniviridae family, and the homolog may be expressed by a second virus in the Coroniviridae family. That is, the family may be Coroniviridae. The microbe expressing the microbial protein may be a coronavirus. One or more of the microbes expressing the homolog may be a coronavirus. All of the microbes expressing the homolog may be a coronavirus. The microbe expressing the microbial protein may be a coronavirus and one or more of microbes expressing the homolog may be a coronavirus. The microbe expressing the microbial protein may be a coronavirus and all of microbes expressing the homolog may be a coronavirus.

The microbe from which the microbial protein is derived and one or more microbes expressing the homolog may be different microbes. That is, the microbe from which the microbial protein is derived may be of a different genus from the one or more microbes expressing the homolog. The microbe from which the microbial protein is derived may be of a different species from the one or more microbes expressing the homolog. The microbe from which the microbial protein is derived may be of a different strain from the one or more microbes expressing the homolog. By way of non-limiting example, the microbial protein may be expressed by SARS-CoV-2 and the homolog may be expressed by one or more non-SARS-CoV-2 coronavirus(es). The non-SARS-CoV-2 coronavirus may, for example, be SARS-CoV-1 or a common cold coronavirus such as HKU1, OC43, 229E and/or NL63.

One or more of the microbes that express the homolog may be endemic within a population. Preferably, each of the one or more microbes that express the homolog is endemic within a population. A pathogen may be defined as endemic in a population when infection with the pathogen is constantly maintained at a baseline level in the population without external inputs. For example, chickenpox is endemic in the United Kingdom population, but malaria is not. The population may be a geographical population. In other words, the population may be defined in terms of the area (e.g. region, country, continent) in which its members reside. The population may be defined in terms of attributes of its members, such as health status, vaccination status, age and so on.

The microbe from which the microbial protein is derived and the microbe expressing the homolog may each be capable of infecting the same species. That is, both the microbe from which the microbial protein is derived and the microbe expressing the homolog may be capable of infecting an individual belonging to a given species. The microbe from which the microbial protein is derived and the microbe expressing the homolog may be capable of infecting the same individual. The microbe from which the microbial protein is derived and the microbe expressing the homolog may be capable of infecting the different individuals belonging to the same species. The species may, for example, be canine, feline, avian, bovine, ovine, equine, porcine, murine or primate. Preferably, the species is human.

One or more (such as two or more, three or more, or four or more) of the microbes expressing the homolog may be an endemic common cold coronavirus. All of the microbes expressing the homolog may be an endemic common cold coronaviruses. For example, the one or more microbes expressing the homolog may comprise (A) HKU1, (B) OC43, (C) 229E and/or (D) NL63. The one or more microbes expressing the homolog may, for example, comprise (A); (B); (C); (D); (A) and (B); (A) and (C); (A) and (D); (B) and (C); (B) and (D); (C) and (D); (A), (B) and (C); (A), (B) and (D); (A), (C) and (D); (B), (C) and (D); or (A), (B), (C) and (D). In any of these cases, the microbe from which the microbial protein is derived may be SARS-CoV-2.

### Step (c) Preparing a Pool of Fragments

Step (c) comprises preparing a pool of fragments in which: (i) each fragment is a fragment identified in step (a) for which step (b) determines the existence of a homolog; or (ii) each fragment is a fragment identified in step (a) for which step (b) does not determine the existence of a homolog, and the fragments form a protein fragment library encompassing at least 80% of the sequence of the microbial protein. Pool of fragments (i) and pool of fragments (ii) are each described in detail in the “Fragments and fragment pools” section above.

Methods for preparing a pool of fragments are well known in the art. In essence, each fragment to be included in the pool is obtained, and the pool is produced by combining each fragment into a single composition. A fragment comprised in the pool may be chemically derived from the parent protein, for example by proteolytic cleavage. A fragment comprised in the pool may be derived in an intellectual sense from the parent protein, for example by making use of the amino acid sequence of the parent protein and synthesising fragments based on the sequence. Fragments may be synthesised using methods well known in the art.

### Pool of Fragments

Disclosed herein is a pool of fragments derived from a microbial protein, wherein: (I) each fragment is comprised in a protein fragment library encompassing at least 80% of the sequence of the microbial protein, and has a homolog that is an amino acid sequence that has at least 60% sequence identity to the fragment and is expressed by one or more microbes in the same family as the microbe from which the microbial protein is derived; or (II) the fragments form a protein fragment library encompassing at least 80% of the sequence of the microbial protein, and each fragment does not have a homolog that is an amino acid sequence that has at least 60% sequence identity to the fragment and is expressed by one or more microbes in the same family as the microbe from which the microbial protein is derived. The pool may, for example, be produced according to the method described above.

Fragments and pools of fragments are described in detail in the section “Fragments and fragment pools” above. Any of the aspects described in that section may apply to the pool of fragments disclosed herein. Microbial proteins are described in detail in the section “Microbial protein” above. Any of the aspects described in that section may apply to the pool of fragments disclosed herein. Further features of pool of fragments (I) and pool of fragments (II) are set out below.

### Pool of Fragments (I)

Each fragment comprised in the pool of fragments (I) is a fragment that is comprised in a protein fragment library encompassing at least 80% of the sequence of the microbial protein. The fragments comprised in the pool of fragments (I) need not themselves form such a protein fragment library. Rather, each fragment comprised in the pool of fragments (I) is a fragment that is notionally comprised in a protein fragment library that encompasses at least 80% of the sequence of the microbial protein. In other words, each fragment comprised in the pool of fragments (I) is a fragment that is or would be found in a protein fragment library that encompasses at least 80% of the sequence of the microbial protein. Protein fragment libraries that encompasses at least 80% of the sequence of the microbial protein are described in detail in the section “—step (a)—identifying fragments comprised in a protein fragment library” above. Any of the aspects described in that section may apply to the pool of fragments (I).

Each fragment comprised in the pool of fragments (I) is also a fragment that has a homolog which is an amino acid sequence that has at least 60% sequence identity to the fragment and is expressed by one or more microbes in the same family as the microbe from which the microbial protein is derived. Such homologs are described in detail in the section “Step (b)—determining the existence of a homolog” above. Any of the aspects described in that section may apply to the pool of fragments (I).

The pool of fragments (I) essentially comprises fragments that are not unique to the microbe from which the microbial protein is derived. The pool of fragments (I) may thus comprise fragments that may be recognised by a cross-reactive immune response. That is, the pool of fragments (I) may comprise fragments that are recognised by (e.g. bind to antigen receptors on and/or trigger a response by) immune cells that are generated by contact with a microbe other that the microbe from which the microbial protein is derived.

### Pool of Fragments (II)

Each fragment comprised in the pool of fragments (II) is a fragment that is identified as being comprised in a protein fragment library encompassing at least 80% of the sequence of the microbial protein. In other words, each fragment comprised in the pool of fragments (II) is a fragment that is notionally comprised in a protein fragment library that encompasses at least 80% of the sequence of the microbial protein. That is, each fragment comprised in the pool of fragments (II) is a fragment that is or would be found in a protein fragment library that encompasses at least 80% of the sequence of the microbial protein. Protein fragment libraries that encompasses at least 80% of the sequence of the microbial protein are described in detail in the section “Step (a)—identifying fragments comprised in a protein fragment library” above. Any of the aspects described in that section may apply to the pool of fragments (II).

In addition, the fragments comprised in the pool (II) themselves form protein fragment library encompassing at least 80% of the sequence of the microbial protein. For example, the fragments comprised in the pool (II) may form a protein fragment library encompassing at least 85%, at least 90%, at least 95%, at least 98%, at least 99% of the sequence of the microbial protein. The fragments comprised in the pool (II) may form a protein fragment library encompassing the entire sequence of the microbial protein. Protein fragment libraries that encompasses at least 80% of the sequence of the microbial protein are described in detail in the section “Step (a)—identifying fragments comprised in a protein fragment library” above. Any of the aspects described in that section may apply to the pool of fragments (II). As explained above, in a protein fragment library in which the fragments together encompass at least 80% (such as at least 80%, at least 85%, at least 90%, at least 95%, at least 98%, at least 99%, or 100%) of the sequence of the microbial protein, the fragments are diverse enough that the pool contains epitopes capable of binding to many different WIC alleles. This allows the pool to be used in assays for cell mediated immunity across the global population, despite variation in WIC alleles between subjects.

In addition, each fragment comprised in the pool of fragments (II) is a fragment that does not have a homolog which is an amino acid sequence that has at least 60% sequence identity to the fragment and is expressed by one or more microbes in the same family as the microbe from which the microbial protein is derived. Such homologs are described in detail in the section “Step (b)—determining the existence of a homolog” above. Any of the aspects described in that section may apply to the pool of fragments (II).

The pool of fragments (II) essentially comprises fragments that are unique to the microbe from which the microbial protein is derived. In other words, the pool of fragments (II) essentially comprises only fragments that do not have a homolog in another microbe belonging to the same family as the microbe from which the microbial protein is derived. Thus, the pool of fragments (II) may exclude fragments that may be recognised by a cross-reactive immune response. That is, the pool of fragments (II) may exclude fragments that are recognised by (e.g. bind to antigen receptors on and/or trigger a response by) immune cells generated by contact with a microbe other that the microbe from which the microbial protein is derived.

### Consolidated Pool of Fragments

Disclosed herein is a consolidated pool of fragments which comprises two or more pools of the present disclosure. Each of the two or more pools comprises fragments derived from a different microbial protein. Each of the two or more pools may be produced according to a method of the present disclosure.

Fragments and pools of fragments are described in detail in the section “Fragments and fragment pools” above. Any of the aspects described in that section may apply to the consolidated pool of fragments disclosed herein. Microbial proteins are described in detail in the section “Microbial protein” above. Any of the aspects described in that section may apply to the consolidated pool of fragments disclosed herein. Further features of the consolidated pool of fragments are set out below.

Each of the two or more pools comprised in the consolidated pool of fragments may be selected from: (I) a pool of fragments derived from a microbial protein, wherein each fragment is comprised in a protein fragment library encompassing at least 80% of the sequence of the microbial protein, and has a homolog that is an amino acid sequence that has at least 60% sequence identity to the fragment and is expressed by one or more microbes in the same family as the microbe from which the microbial protein is derived; and (II) a pool of fragments derived from a microbial protein, wherein the fragments form a protein fragment library encompassing at least 80% of the sequence of the microbial protein, and each fragment does not have a homolog that is an amino acid sequence that has at least 60% sequence identity to the fragment and is expressed by one or more microbes in the same family as the microbe from which the microbial protein is derived.

The consolidated pool may comprise both: (I) a pool of fragments derived from a microbial protein, wherein each fragment is comprised in a protein fragment library encompassing at least 80% of the sequence of the microbial protein, and has a homolog that is an amino acid sequence that has at least 60% sequence identity to the fragment and is expressed by one or more microbes in the same family as the microbe from which the microbial protein is derived; and (II) a pool of fragments derived from a microbial protein, wherein the fragments form a protein fragment library encompassing at least 80% of the sequence of the microbial protein, and each fragment does not have a homolog that is an amino acid sequence that has at least 60% sequence identity to the fragment and is expressed by one or more microbes in the same family as the microbe from which the microbial protein is derived.

The consolidated pool may comprise either: (I) a pool of fragments derived from a microbial protein, wherein each fragment is comprised in a protein fragment library encompassing at least 80% of the sequence of the microbial protein, and has a homolog that is an amino acid sequence that has at least 60% sequence identity to the fragment and is expressed by one or more microbes in the same family as the microbe from which the microbial protein is derived; or (II) a pool of fragments derived from a microbial protein, wherein the fragments form a protein fragment library encompassing at least 80% of the sequence of the microbial protein, and each fragment does not have a homolog that is an amino acid sequence that has at least 60% sequence identity to the fragment and is expressed by one or more microbes in the same family as the microbe from which the microbial protein is derived. Thus, the consolidated pool may comprise two or more pools according to (I) and no pools according to (II). The consolidated pool may comprise two or more pools according to (II) and no pools according to (I).

Each of the two or more pools comprised in the consolidated pool comprises fragments derived from a different microbial protein. Inclusion of pools comprising fragments derived from a different microbial protein increases the likelihood of eliciting a cell mediated immune response when the consolidated pool is used in an assay for cell mediated immunity. Preferably, each of the two or more pools comprises fragments derived from a different microbial protein expressed by the same microbe. For example, each of the two or more pools may comprise fragments derived from a different microbial protein expressed by the same coronavirus. Each of the two or more pools may comprise fragments derived from a different microbial protein expressed by SARS-CoV-2. For instance, each of the two or more pools may comprise fragments derived from a different microbial protein selected from (A) SARS-CoV-2 S1 spike domain, (B) SARS-CoV-2 S2 spike domain, (C) SARS-CoV-2 nucleocapsid protein, (D) SARS-CoV-2 membrane protein/or (E) SARS-CoV-2 envelope protein. The consolidated pool may, for example, comprise pools of fragments derived from (A) and (B); (A) and (C); (A) and (D) (A) and (E); (B) and (C); (B) and D); (B) and (E); (C) and (D); (C) and (E); (D) and (E); (A), (B) and (C); (A), (B and (D); (A), (B) and (E); (A), (C)) and (D); (A) (C) and (E); (A), (D) and (E); (B), (C) and (D); (B), (C) and (E); (B), (D) and (E); (C), (D) and (E); (A), (B), (C) and (D); (A), (B), (C) and (E); (A), (B), (D) and (E); (A), (C), (D) and (E); (B), (C), (D) and (E); (A), (B), (C), (D) and (E).

For example, the pool may comprise or consist panel 13 (P13) of the Examples. The fragments comprised in P13 are set out in Table 3 in Example 2. P13 is a consolidated pool that comprises four pools that are each (I) a pool of fragments derived from a microbial protein, wherein each fragment is comprised in a protein fragment library encompassing at least 80% of the sequence of the microbial protein, and has a homolog that is an amino acid sequence that has at least 60% sequence identity to the fragment and is expressed by one or more microbes in the same family as the microbe from which the microbial protein is derived. The four pools are derived from (A) SARS-CoV-2 S1 spike domain, (B) SARS-CoV-2 S2 spike domain, (C) SARS-CoV-2 nucleocapsid protein and (D) SARS-CoV-2 membrane protein respectively.

### Method for Determining the Presence or Absence of Immune Cells

Disclosed herein is a method for determining the presence or absence of immune cells targeting a microbe. The method comprises contacting a sample comprising immune cells with one or more fragment pools disclosed herein, and detecting in vitro the presence or absence of an immune response to the one or more pools. The method may comprise an assay for cell-mediated immunity, such as T cell-mediated immunity.

### Sample

The method comprises contacting a sample comprising immune cells with one or more fragment pools disclosed herein. The sample may be a sample that has been obtained from a subject. The subject may be canine, feline, avian, bovine, ovine, equine, porcine, murine or primate. Preferably, the subject is human.

The sample may, for example, comprise whole blood. The sample may comprise immune cells isolated from whole blood. For example, the sample may comprise peripheral blood mononuclear cells (PBMCs) isolated from whole blood. The sample may, for example, comprise T cells. The T cells may comprise CD8+ T cells and/or CD4+ T cells.

Accordingly, the immune cells comprised in the sample may comprise PBMCs. The immune cells comprised in the sample may comprise T cells. The immune cells comprised in the sample may comprise CD8+ T cells. The immune cells comprised in the sample may comprise CD4+ T cells. The immune cells comprised in the sample may comprise CD4+ T cells and CD8+ T cells.

### Fragment Pools

The method comprises contacting a sample comprising immune cells with one or more fragment pools disclosed herein. Such fragment pools are described in detail above.

The sample may, for example, be contacted with two or more fragment pools disclosed herein. For instance, the sample may be contacted with three or more, four or more, or five or more fragment pools disclosed herein.

The one or more fragment pools contacted with the sample may comprise (a) one or more pools of fragments according to pool of fragments (I) described above. For example, the one or more pools contacted with the sample may comprise two or more, three or more, four or more, or five or more pools of fragments according to pool of fragments (I) described above. The one or more pools contacted with the sample may comprise (b) one or more pools of fragments according to pool of fragments (II) described above. For example, the one or more pools contacted with the sample may comprise two or more, three or more, four or more, or five or more pools of fragments according to pool of fragments (II) described above. The one or more pools contacted with the sample may comprise (c) one or more pools of fragments according to the consolidated pool of fragments described above. For example, the one or more pools contacted with the sample may comprise two or more, three or more, four or more, or five or more pools of fragments according to the consolidated pool of fragments described above. The one or more pools contacted with the sample may comprise: (a); (b); (c); (a) and (b); (a) and (c); (b) and (c); or (a), (b) and (c).

When the one or more fragment pools comprises two or more fragment pools, each of the two or more pools may comprise fragments derived from a different microbial protein. That is, the microbial protein from which the fragments in one of the two or more pools are derived may be different from the microbial protein(s) from which the fragments in the other pool(s) are derived. Use of multiple pools each comprising fragments derived from a different microbial protein increases the likelihood of eliciting an immune response by the immune cells comprised in the sample.

Preferably, each of the two or more pools comprises fragments derived from a different microbial protein expressed by the same microbe. For example, each of the two or more pools may comprise fragments derived from a different microbial protein expressed by the same coronavirus. Each of the two or more pools may comprise fragments derived from a different microbial protein expressed by SARS-CoV-2. For instance, each of the two or more pools may comprise fragments derived from a different microbial protein selected from (A) SARS-CoV-2 surface glycoprotein, (B) SARS-CoV-2 nucleocapsid protein, (C) SARS-CoV-2 membrane protein and/or (D) SARS-CoV-2 envelope protein. The two or more pools may, for example, comprise pools of fragments derived from (A) and (B); (A) and (C); (A) and (D); (B) and (C); (B) and (D); (C) and (D); (A), (B) and (C); (A), (B) and (D); (A), (C) and (D); (B), (C) and (D); or (A), (B), (C) and (D). Each of the two or more pools may be contacted with the sample in a separate reaction.

The method may further comprise contacting the sample with a pool of fragments derived from a protein from the microbe, and detecting in vitro the presence or absence of an immune response to the pool, wherein the fragments in the pool form a protein fragment library encompassing at least 80% of the sequence of the protein. Protein fragment libraries that encompasses at least 80% of the sequence of the microbial protein are described—in detail in the section “Step (a)—identifying fragments comprised in a protein fragment library” above. Any of the aspects described in that section may apply to this further pool of fragments. This further pool may comprise fragments capable of stimulating both cell mediated immunity that is cross-reactive for the microbe of interest, and cell mediated immunity that is specific for the microbe of interest. Essentially, this further pool is not specially optimised for use in an assay for cell mediated immunity, and may be used in combination with a pool described herein that is optimised for assaying (I) cell mediated immunity that is cross-reactive for the microbe of interest, or (II) cell mediated immunity that is specific for the microbe of interest. This further contacting step may be conducted in a separate reaction.

The further pool and the one or more pools contacted with the sample may comprise fragments derived from a different microbial protein. Preferably, the further pool and the one or more pools contacted with the sample comprise fragments derived from a different microbial protein expressed by the same microbe. For example, the further pool and the one or more pools contacted with the sample may comprise fragments derived from a different microbial protein expressed by the same coronavirus. Each of the further pool and the one or more pools contacted with the sample may comprise fragments derived from a different microbial protein expressed by SARS-CoV-2. For instance, each of the further pool and the one or more pools contacted with the sample may comprise fragments derived from a different microbial protein selected from (A) SARS-CoV-2 surface glycoprotein, (B) SARS-CoV-2 nucleocapsid protein, (C) SARS-CoV-2 membrane protein and/or (D) SARS-CoV-2 envelope protein. The further pool and the one or more pools contacted with the sample may, for example, comprise pools of fragments derived from (A) and (B); (A) and (C); (A) and (D); (B) and (C); (B) and (D); (C) and (D); (A), (B) and (C); (A), (B) and (D); (A), (C) and (D); (B), (C) and (D); or (A), (B), (C) and (D).

The method may further comprise contacting the sample with a pool of fragments derived from a protein from a microbe in the same family as the microbe from which the microbial protein is derived and detecting in vitro the presence or absence of an immune response to the pool, wherein the fragments in the pool form a protein fragment library encompassing at least 80% of the sequence of the protein. Protein fragment libraries that encompasses at least 80% of the sequence of the microbial protein are described—in detail in the section “Step (a)—identifying fragments comprised in a protein fragment library” above. This further contacting step is conducted in a separate reaction. Preferably, the microbe from which the microbial protein is derived is an emerging pathogen, and the microbe in the same family is endemic within a population. In this case, the further contacting and detecting step provides information about prior exposure to endemic pathogens. This information may aid in the interpretation of an immune response detected in connection with the emerging pathogen. For example, absence of an immune response to the endemic pathogen may help to demonstrate that an immune response detected to the emerging pathogen is specific for that emerging pathogen and not the result of cross-protective immunity conferred by prior exposure to the endemic pathogen.

### Detecting In Vitro the Presence or Absence of an Immune Response

The method comprises detecting in vitro the presence or absence of an immune response to the one or more pools. Mechanisms for detecting in vitro the presence or absence of an immune response are well known in the art.

Detecting the presence or absence of an immune response may, for example, comprise one or more of the following, in any combination:
Determining the number or proportion of cells comprised in the cell sample or an aliquot thereof that are responsive to the one or more pools.
Determining the expression or secretion of one or more cytokines by immune cell comprised in the sample in response to the one or more pools. The one or more cytokines may, for example, comprise interferon gamma (IFNγ).
Determining the number or proportion of immune cells comprised in the sample or an aliquot thereof that secrete one or more cytokines in response to the one or more pools. The one or more cytokines may, for example, comprise interferon gamma (IFNγ).
Determining the expression of one or more markers by immune cells comprised in the sample in response to the one or more pools. The immune cells may comprise T cells. The one or more markers may, for example, comprised markers of activation, degranulation, or other T cell functions. T cell markers and their associated functions are well known in the art.
   Methods for such determination are known in the art.

Detecting in vitro the presence or absence of an immune response may, for example, comprise determining the number or proportion of immune cells comprised in the cell sample or an aliquot thereof that are responsive to the one or more pools. This may comprise determining the number or proportion of immune cells comprised in the cell sample or an aliquot thereof that secrete one or more cytokines in response to the one or more pools. The cytokine may, for example, be interferon gamma (IFNγ). Methods for such determination are well known in the art and include, for example, flow cytometry and ELISpot assays. Preferably, such determination is by enzyme-linked immunospot (ELISpot) assay.

The method may, for example, comprise an interferon gamma release assay (IGRA). Assays for interferon gamma release are well-known in the art and include, for example, ELISpot assays and enzyme linked immunosorbent assays (ELISA), such as in-tube ELISAs.

Preferably, the method comprises an ELISpot assay. Preferably, the ELISpot assay is an interferon gamma release assay (IGRA). Preferably, the ELISpot assay is an interferon gamma release assay (IGRA) and the immune cells comprise T cells, such as CD8+ T cells and/or CD4+ T cells.

ELISpot assays are well-known in the art. The ELISpot is an immunoassay that measures the frequency of protein secreting cells in a sample at the single-cell level. Cells from the cell sample are cultured in one or more wells of an assay plate. Cells may be cultured at a density of, for example, 100,000 to 500,000 cells per well. For instance, cells may be cultured at a density of 150,000 to 450,000 cells per well; 200,000 to 400,000 cells per well; 250,000 to 350,000 cells per well. For example, cells may be cultured at a density of about 100,000, about 150,000, about 200,000, about 250,000, about 300,000, about 350,000, about 400,000, about 450,000 or about 500,000 cells per well. Cells are preferably cultured at a density of about 250,000 cells per well. Each well comprises a surface coated with a capture antibody specific for the secreted protein of interest. A different stimulus regime may be applied to each of the one or more well, for example to provide test wells and control wells. Proteins that are secreted by the cells are captured by the capture antibody. After an appropriate incubation time, cells are removed and the secreted protein is detected using a detection antibody that is directly or indirectly conjugated with an enzyme. Upon contact of the enzyme with a substrate forming precipitating product, visible spots from on the surface. Each spot corresponds to an individual protein-secreting cell. The assay is interpreted based on number of spots formed in each well. Spot count may be expressed as <number of spots> per <number of cultured cells>, or a multiple thereof. For example, if 250,000 cells are cultured in each well, spot count may be expressed as spots per 250,000 cells or a multiple thereof (e.g. spots per million cells).

The method may comprise conducting one or more separate reactions in order to contact each pool with a different aliquot of the cell sample. Preferably, each of the different aliquots has substantially the same composition. An aliquot is essentially a divided portion of the cell sample. Contacting each pool with a different aliquot of the cell sample allows the sample to be contacted with each of the pools separately. In other words, the sample can be contacted with each pool in a physically separate reaction. A plurality of physically separate reactions may be performed in order to contact each of a plurality of aliquots with a different pool. The physically separate reactions are preferably performed at the same time. When the method comprises an ELISpot assay, the physically separate reactions may, for example, be performed in different wells of an ELISpot plate.

In addition to the separate reactions conducted to contact each pool with a different aliquot of the cell sample, the method may comprise conducting one or more separate reactions in order to provide a negative control reaction or a positive control reaction. A negative control reaction may, for example, comprise an aliquot of the cell sample in the absence of a pool of fragments or other antigen. A positive control reaction may, for example, comprise an aliquot of the cell sample and a known stimulator of cells comprised in the cell sample. When the cell sample comprises T cells, the known stimulator may for example be phytohaemagglutinin (PHA).

It is readily apparent to the skilled person how the presence or absence of an immune response to the one or more pools may be detected based on the various determinations described above. For example:
The presence of cells in the sample that are responsive to the one or more pools may indicate the presence of an immune response to the one or more pools. The absence of cells in the sample that are responsive to the one or more pools may, for example, indicate the absence of an immune response to the one or more pools.
Expression or secretion of one or more cytokines by immune cells comprised in the sample in response to the one or more pools may, for example, indicate the presence of an immune response to the one or more pools. The absence of expression or secretion of one or more cytokines by immune cells comprised in the sample in response to the one or more pools may, for example, indicate the absence of an immune response to the one or more pools.
The number or proportion of immune cells comprised in the sample or an aliquot thereof that secrete one or more cytokines in response to one or more pools may, for example, indicate the presence or absence of an immune response to the one or more pools. That is, the presence or absence of an immune response to the one or more pools may be determined based on the number of immune cells comprised in the sample or an aliquot thereof that secrete one or more cytokines in response to the one or more pools. The presence or absence of an immune response may be determined based on the proportion of immune cells comprised in the sample or an aliquot thereof that secrete one or more cytokines in response to the one or more pools.
The expression of one or more markers by one or more immune cells comprised in the sample in response to one or more pools may indicate the presence of an immune response to the one or more pools. The absence of expression of one or more markers by one or more immune cells comprised in the sample in response to the one or more pools may indicate the absence of an immune response to the one or more pools.

When the method comprises an ELISpot assay, detecting the presence or absence of an immune response to the one or more pools may comprise determining the number of spots formed in each well. Detecting the presence or absence of an immune response to the one or more pools may comprise processing mathematically the number of spots formed in each well (for example by calculating the square root of the number of spots, the cubic root of the number of spots, and/or log(<number of spots>+1)). A cut-off may be applied to the number of spots formed in each well (or the mathematically processed equivalent thereof) in order to determine the presence or absence of an immune response to the one or more pools.

In one aspect disclosed herein, the method may further comprise the step of diagnosing the presence or absence of infection with the microbe in a subject from which the sample is obtained. That is, the method for determining the presence of absence of immune cells targeting a microbe may be a method for determining the presence or absence of infection with the microbe. The method for determining the presence or absence of immune cells targeting a microbe may be a method for diagnosing infection with the microbe. The presence of an immune response to the one or more pools may indicate the presence of infection with the microbe in the subject. The absence of an immune response to the one or more pools may indicate the absence of infection with the microbe in the subject.

The following Examples illustrate the invention.

### Example 1—SARS-CoV-2 Peptide Pool Bioinformatics Homology Search

### Objectives

Analyse peptide sequences generated from the main structural proteins of SARS-CoV-2 for homology to any common human pathogen using a bioinformatics approach.

### Summary

Significant homology was detected between SARS-CoV-2 peptides and various human coronaviruses, including SARS-CoV-1 and the endemic common cold coronaviruses. Modified peptide lists can be generated by removing peptide with detected homology.

### 1. Introduction/Background

T-SPOT Discovery SARS-CoV-2 is an assay kit for studying the immune response to SARS-CoV-2, the causative agent of COVID-19. T-SPOT Discovery SARS-CoV-2 consists of pools of overlapping 15-mer peptides which scan the full length of the four major structural proteins of SARS-CoV-2. These proteins are the spike surface glycoprotein (S or spike; which comprises S1 spike domain and S2 spike domain), the nucleocapsid phosphoprotein (N or nuc), the membrane glycoprotein (M or memb) and the envelope protein (env or E).

As SARS-CoV-2 is an emerging human pathogen, the immune response to the virus has not been fully characterised. SARS-CoV-2-specific CD4 and CD8 T-cells have been identified in recovered patients. In these studies, SARS-CoV-2 T-cell responses were also detected in donor samples isolated before the emergence of the virus. This suggests that there is some level of cross-reactive immune response, possibly originally targeting the endemic common cold human coronaviruses.

This study utilised a bioinformatics approach to characterise overlapping peptide panels generated from the main structural proteins of SARS-CoV-2. Homology to other human pathogens was assessed by homology alignment search using the BLAST search engine.

### 2. Results

### 2.1. Overlapping Peptide Generation

The following Genbank accession numbers were used for the reference sequences of the SARS-CoV-2 proteins: surface glycoprotein—qhd43416.1, nucleocapsid—qhd43423.2, membrane—qhd43419.1, and envelope—yp_009724392.1. See appendix 1 for full protein sequences. Amino acids 1 to 643 of qhd43416.1 (SEQ ID NO: 741) represent S1 spike domain. Amino acids 633 to 1273 of qhd43416.1 (SEQ ID NO: 741) represent S2 spike domain.

Four lists of 15-mer peptide with 11-aa overlap sequences were generated (appendix 2).

### 2.2. Homology Search

The 487 peptide sequences generated in section 2.1 were searched for homology using the BLAST search tool. Approximately 50,000 results were retrieved from the searches.

Results were filtered by number of matching amino acids between the peptide sequence and the result sequence, with greater than or equal to 9 matches considered high homology. This method fails to filter out matches consisting of multiple small alignments (e.g. three separate alignments of three residues) but does capture all high homology matches.

Five main categories of homology matches were detected:
1. SARS-CoV-2. These results were expected and confirm the correct sequences were used for the search terms
2. SARS-CoV-1. SARS-CoV-2 shares a very high level of homology with SARS-CoV-1. Approximately 400 peptides from the 487 peptides on the list have detectable homology to SARS-CoV-1.
3. Non-coronavirus human pathogens. No major human pathogens or antigens were detected in the homology search. Several low quality hits (E values>1) were detected against pathogens such as E. coli and Campylobacter proteins, however these are unlikely have cross-reactive immune responses as the homology is quite low.
4. Animal coronaviruses. There were over 1000 matches to 130 unique proteins from more than 50 different animal coronaviruses. Table 1 lists the animal coronaviruses detected. Despite the high homology detected between SARS-CoV-2 and the animal coronaviruses these sequences are unlikely to cause cross-reactive immune responses as it is very unlikely that humans would have been exposed to these viruses.

TABLE 1
Animal coronaviruses with significant homology to SARS-CoV-2 peptides

| | | |
|---|---|---|
| Betacoronavirus | Pipistrellus bat | Mink coronavirus strain |
| Erinaceus/VMC/DEU/2012 | coronavirus HKU5 | WD1127 |
| Bat coronavirus BM48- | Rousettus bat coronavirus | Munia coronavirus |
| 31/BGR/2008 | HKU9 | HKU13-3514 |
| Bat Hp- | Tylonycteris bat | Rat coronavirus Parker |
| betacoronavirus/Zhejiang | coronavirus HKU4 | |
| 2013 | | |
| Magpie-robin coronavirus | Bat coronavirus 1A | Rodent coronavirus |
| HKU18 | | |
| Rabbit coronavirus | Betacoronavirus HKU24 | Rousettus bat coronavirus |
| HKU14 | | HKU10 |
| White-eye coronavirus | Canada goose coronavirus | Rousettus bat coronavirus |
| HKU16 | | |
| Wigeon coronavirus | Coronavirus AcCoV- | Shrew coronavirus |
| HKU20 | JC34 | |
| Bovine coronavirus | Ferret coronavirus | Swine enteric coronavirus |
| Scotophilus bat | Lucheng Rn rat | Thrush coronavirus |
| coronavirus 512 | coronavirus | HKU12-600 |
| Turkey coronavirus | Camel alphacoronavirus | Bulbul coronavirus |
| | | HKU11-934 |
| Betacoronavirus | Feline infectious | Porcine coronavirus |
| England 1 | peritonitis virus | HKU15 |
| NL63-related bat | Infectious bronchitis virus | Sparrow coronavirus |
| coronavirus | | HKU17 |
| Rhinolophus bat | Murine hepatitis virus | Alphacoronavirus . . . |
| coronavirus HKU2 | | |
| Murine hepatitis virus | Porcine epidemic diarrhea | Beluga whale coronavirus |
| strain JHM | virus | SW1 |
| Alphacoronavirus . . . | Wencheng Sm shrew | Miniopterus bat |
| | coronavirus | coronavirus HKU8 |
| BtMr- | Middle East respiratory | Bat coronavirus |
| AlphaCoV/SAX2011 | syndrome-related . . . | CDPHE15/USA/2006 |
| BtNv-AlphaCoV/SC2013 | Common moorhen | BtRf-AlphaCoV/YN2012 |
| | coronavirus HKU21 | |
| BtRf- | Night heron coronavirus | Transmissible |
| AlphaCoV/HuB2013 | HKU19 | gastroenteritis virus |

5. Endemic human coronaviruses. Multiple matches to all four endemic human coronaviruses (HKU1, OC43, 229E, NL63) were detected. Table 2 lists the proteins and viruses where homology was detected. Homology was detected in 26 peptides from the spike, membrane and nucleocapsid pools. Homology was not detected in any peptides from the envelope pool. Appendix 3(a) lists the sequences of the peptides with high homology to the endemic human coronaviruses. The endemic human coronaviruses are a likely source of any cross reactive immune response as infection with these viruses are very common. To ensure that all homology with the endemic human coronaviruses was captured the filtering criteria was removed and all human coronavirus hits were selected from the BLAST results. This gave a list of 46 peptides with homology to the human coronavirus. Appendix 3(b) list these sequences.

TABLE 2
Human coronaviruses and proteins with significant
homology to SARS-CoV-2 peptides

Membrane glycoprotein [Human coronavirus HKU1]
Membrane protein [Human coronavirus OC43]
Nucleocapsid phosphoprotein [Human coronavirus HKU1]
Nucleocapsid protein [Human coronavirus 229E]
Nucleocapsid protein [Human coronavirus OC43]
Spike glycoprotein [Human coronavirus HKU1]
Spike protein [Human coronavirus NL63]
Spike surface glycoprotein [Human coronavirus OC43]
Surface glycoprotein [Human coronavirus 229E]

### 3. Conclusion

Sequences for 487 overlapping peptides were generated from the spike, membrane, nucleocapsid and envelop proteins of SARS-CoV-2. Homology to common human pathogens was detected by performing a BLAST search on the sequences. The pathogens with the highest homology to the SARS-CoV-2 peptides were SARS-CoV-1 and the endemic human coronaviruses. The potential for peptide pools to provoke a cross-reactive immune responses could be reduced by removing the identified peptides from the antigen pools used in a SARS-CoV-2 assay, such as an assay for cell mediated immunity to SARS-CoV-2.

### APPENDIX 1—FULL PROTEIN SEQUENCES

### Full Protein Sequence of SARS-CoV-2 Surface Glycoprotein (Spike Glycoprotein) [QHD43416.1]

(SEQ ID NO: 741)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

### Full Protein Sequence of SARS-CoV-2 Membrane Glycoprotein [QHD43419.1]

(SEQ ID NO: 742)
MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ

### Full Protein Sequence of SARS-CoV-2 Nucleocapsid Phosphoprotein [QHD43423.2]

(SEQ ID NO: 743)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVILLPAADL
DDFSKQLQQSMSSADSTQA

### Full Protein Sequence of SARS-CoV-2 Envelope Protein [YP_009724392.1]

(SEQ ID NO: 744)
MYSFVSEETGTLIVNSVLLFLAFVVFLLVTLAILTALRLCAYCCNIVNVS
LVKPSFYVYSRVKNLNSSRVPDLLV

### APPENDIX 2—OVERLAPPING PEPTIDE SEQUENCES

Overlapping Peptide Sequences Derived from SARS-CoV-2 Surface Glycoprotein (Spike Glycoprotein) [Qhd43416.1]

| | SEQ ID | | SEQ ID | | SEQ ID |
|---|---|---|---|---|---|
| Fragment | NO: | Fragment | NO: | Fragment | NO: |
| MFVFLVLLPLVSSQC | 1 | YNYKLPDDFTGCVIA | 106 | LGDIAARDLICAQKF | 211 |
| LVLLPLVSSQCVNLT | 2 | LPDDFTGCVIAWNSN | 107 | AARDLICAQKFNGLT | 212 |
| PLVSSQCVNLTTRTQ | 3 | FTGCVIAWNSNNLDS | 108 | LICAQKFNGLTVLPP | 213 |
| SQCVNLTTRTQLPPA | 4 | VIAWNSNNLDSKVGG | 109 | QKFNGLTVLPPLLTD | 214 |
| NLTTRTQLPPAYTNS | 5 | NSNNLDSKVGGNYNY | 110 | GLTVLPPLLTDEMIA | 215 |
| RTQLPPAYTNSFTRG | 6 | LDSKVGGNYNYLYRL | 111 | LPPLLTDEMIAQYTS | 216 |
| PPAYTNSFTRGVYYP | 7 | VGGNYNYLYRLFRKS | 112 | LTDEMIAQYTSALLA | 217 |
| TNSFTRGVYYPDKVF | 8 | YNYLYRLFRKSNLKP | 113 | MIAQYTSALLAGTIT | 218 |
| TRGVYYPDKVFRSSV | 9 | YRLFRKSNLKPFERD | 114 | YTSALLAGTITSGWT | 219 |
| YYPDKVFRSSVLHST | 10 | RKSNLKPFERDISTE | 115 | LLAGTITSGWTFGAG | 220 |
| KVFRSSVLHSTQDLF | 11 | LKPFERDISTEIYQA | 116 | TITSGWTFGAGAALQ | 221 |
| SSVLHSTQDLFLPFF | 12 | ERDISTEIYQAGSTP | 117 | GWTFGAGAALQIPFA | 222 |
| HSTQDLFLPFFSNVT | 13 | STEIYQAGSTPCNGV | 118 | GAGAALQIPFAMQMA | 223 |
| DLFLPFFSNVTWFHA | 14 | YQAGSTPCNGVEGEN | 119 | ALQIPFAMQMAYRFN | 224 |
| PFFSNVTWFHAIHVS | 15 | STPCNGVEGFNCYFP | 120 | PFAMQMAYRFNGIGV | 225 |
| NVTWFHAIHVSGTNG | 16 | NGVEGFNCYFPLQSY | 121 | QMAYRFNGIGVTQNV | 226 |
| FHAIHVSGTNGTKRF | 17 | GFNCYFPLQSYGFQP | 122 | RFNGIGVTQNVLYEN | 227 |
| HVSGTNGTKRFDNPV | 18 | YFPLQSYGFQPTNGV | 123 | IGVTQNVLYENQKLI | 228 |
| TNGTKRFDNPVLPFN | 19 | QSYGFQPTNGVGYQP | 124 | QNVLYENQKLIANQF | 229 |
| KRFDNPVLPFNDGVY | 20 | FQPTNGVGYQPYRVV | 125 | YENQKLIANQFNSAI | 230 |
| NPVLPFNDGVYFAST | 21 | NGVGYQPYRVVVLSF | 126 | KLIANQFNSAIGKIQ | 231 |
| PFNDGVYFASTEKSN | 22 | YQPYRVVVLSFELLH | 127 | NQFNSAIGKIQDSLS | 232 |
| GVYFASTEKSNIIRG | 23 | RVVVLSFELLHAPAT | 128 | SAIGKIQDSLSSTAS | 233 |
| ASTEKSNIIRGWIFG | 24 | LSFELLHAPATVCGP | 129 | KIQDSLSSTASALGK | 234 |
| KSNIIRGWIFGTTLD | 25 | LLHAPATVCGPKKST | 130 | SLSSTASALGKLQDV | 235 |
| IRGWIFGTTLDSKTQ | 26 | PATVCGPKKSTNLVK | 131 | TASALGKLQDVVNQN | 236 |
| IFGTTLDSKTQSLLI | 27 | CGPKKSTNLVKNKCV | 132 | LGKLQDVVNQNAQAL | 237 |
| TLDSKTQSLLIVNNA | 28 | KSTNLVKNKCVNFNF | 133 | QDVVNQNAQALNTLV | 238 |
| KTQSLLIVNNATNVV | 29 | LVKNKCVNFNFNGLT | 134 | NQNAQALNTLVKQLS | 239 |
| LLIVNNATNVVIKVC | 30 | KCVNFNFNGLTGTGV | 135 | QALNTLVKQLSSNFG | 240 |
| NNATNVVIKVCEFQF | 31 | FNFNGLTGTGVLTES | 136 | TLVKQLSSNFGAISS | 241 |
| NVVIKVCEFQFCNDP | 32 | GLTGTGVLTESNKKF | 137 | QLSSNFGAISSVLND | 242 |
| KVCEFQFCNDPFLGV | 33 | TGVLTESNKKFLPFQ | 138 | NFGAISSVLNDILSR | 243 |
| FQFCNDPFLGVYYHK | 34 | TESNKKFLPFQQFGR | 139 | ISSVLNDILSRLDKV | 244 |
| NDPFLGVYYHKNNKS | 35 | KKFLPFQQFGRDIAD | 140 | LNDILSRLDKVEAEV | 245 |
| LGVYYHKNNKSWMES | 36 | PFQQFGRDIADTTDA | 141 | LSRLDKVEAEVQIDR | 246 |
| YHKNNKSWMESEFRV | 37 | FGRDIADTTDAVRDP | 142 | DKVEAEVQIDRLITG | 247 |
| NKSWMESEFRVYSSA | 38 | IADTTDAVRDPQTLE | 143 | AEVQIDRLITGRLQS | 248 |
| MESEFRVYSSANNCT | 39 | TDAVRDPQTLEILDI | 144 | IDRLITGRLQSLQTY | 249 |
| FRVYSSANNCTFEYV | 40 | RDPQTLEILDITPCS | 145 | ITGRLQSLQTYVTQQ | 250 |
| SSANNCTFEYVSQPF | 41 | TLEILDITPCSFGGV | 146 | LQSLQTYVTQQLIRA | 251 |
| NCTFEYVSQPFLMDL | 42 | LDITPCSFGGVSVIT | 147 | QTYVTQQLIRAAEIR | 252 |
| EYVSQPFLMDLEGKQ | 43 | PCSFGGVSVITPGTN | 148 | TQQLIRAAEIRASAN | 253 |
| QPFLMDLEGKQGNFK | 44 | GGVSVITPGTNTSNQ | 149 | IRAAEIRASANLAAT | 254 |
| MDLEGKQGNFKNLRE | 45 | VITPGTNTSNQVAVL | 150 | EIRASANLAATKMSE | 255 |
| GKQGNFKNLREFVFK | 46 | GTNTSNQVAVLYQDV | 151 | SANLAATKMSECVLG | 256 |
| NFKNLREFVFKNIDG | 47 | SNQVAVLYQDVNCTE | 152 | AATKMSECVLGQSKR | 257 |
| LREFVFKNIDGYFKI | 48 | AVLYQDVNCTEVPVA | 153 | MSECVLGQSKRVDFC | 258 |
| VFKNIDGYFKIYSKH | 49 | QDVNCTEVPVAIHAD | 154 | VLGQSKRVDFCGKGY | 259 |
| IDGYFKIYSKHTPIN | 50 | CTEVPVAIHADQLTP | 155 | SKRVDFCGKGYHLMS | 260 |
| FKIYSKHTPINLVRD | 51 | PVAIHADQLTPTWRV | 156 | DFCGKGYHLMSFPQS | 261 |
| SKHTPINLVRDLPQG | 52 | HADQLTPTWRVYSTG | 157 | KGYHLMSFPQSAPHG | 262 |
| PINLVRDLPQGFSAL | 53 | LTPTWRVYSTGSNVF | 158 | LMSFPQSAPHGVVFL | 263 |
| VRDLPQGFSALEPLV | 54 | WRVYSTGSNVFQTRA | 159 | PQSAPHGVVFLHVTY | 264 |
| PQGFSALEPLVDLPI | 55 | STGSNVFQTRAGCLI | 160 | PHGVVFLHVTYVPAQ | 265 |
| SALEPLVDLPIGINI | 56 | NVFQTRAGCLIGAEH | 161 | VFLHVTYVPAQEKNF | 266 |
| PLVDLPIGINITRFQ | 57 | TRAGCLIGAEHVNNS | 162 | VTYVPAQEKNFTTAP | 267 |
| LPIGINITRFQTLLA | 58 | CLIGAEHVNNSYECD | 163 | PAQEKNFTTAPAICH | 268 |
| INITRFQTLLALHRS | 59 | AEHVNNSYECDIPIG | 164 | KNFTTAPAICHDGKA | 269 |
| RFQTLLALHRSYLTP | 60 | NNSYECDIPIGAGIC | 165 | TAPAICHDGKAHFPR | 270 |
| LLALHRSYLTPGDSS | 61 | ECDIPIGAGICASYQ | 166 | ICHDGKAHFPREGVF | 271 |
| HRSYLTPGDSSSGWT | 62 | PIGAGICASYQTQTN | 167 | GKAHFPREGVFVSNG | 272 |
| LTPGDSSSGWTAGAA | 63 | GICASYQTQTNSPRR | 168 | FPREGVFVSNGTHWF | 273 |
| DSSSGWTAGAAAYYV | 64 | SYQTQTNSPRRARSV | 169 | GVFVSNGTHWFVTQR | 274 |
| GWTAGAAAYYVGYLQ | 65 | QTNSPRRARSVASQS | 170 | SNGTHWFVTQRNFYE | 275 |
| GAAAYYVGYLQPRTF | 66 | PRRARSVASQSIIAY | 171 | HWFVTQRNFYEPQII | 276 |
| YYVGYLQPRTFLLKY | 67 | RSVASQSIIAYTMSL | 172 | TQRNFYEPQIITTDN | 277 |
| YLQPRTFLLKYNENG | 68 | SQSIIAYTMSLGAEN | 173 | FYEPQIITTDNTFVS | 278 |
| RTFLLKYNENGTITD | 69 | IAYTMSLGAENSVAY | 174 | QIITTDNTFVSGNCD | 279 |
| LKYNENGTITDAVDC | 70 | MSLGAENSVAYSNNS | 175 | TDNTFVSGNCDVVIG | 280 |
| ENGTITDAVDCALDP | 71 | AENSVAYSNNSIAIP | 176 | FVSGNCDVVIGIVNN | 281 |
| ITDAVDCALDPLSET | 72 | VAYSNNSIAIPTNFT | 177 | NCDWVIGIVNNTVYD | 282 |
| VDCALDPLSETKCTL | 73 | NNSIAIPTNFTISVT | 178 | VIGIVNNTVYDPLQP | 283 |
| LDPLSETKCTLKSFT | 74 | AIPTNFTISVTTEIL | 179 | VNNTVYDPLQPELDS | 284 |
| SETKCTLKSFTVEKG | 75 | NFTISVTTEILPVSM | 180 | VYDPLQPELDSFKEE | 285 |
| CTLKSFTVEKGIYQT | 76 | SVTTEILPVSMTKTS | 181 | LQPELDSFKEELDKY | 286 |
| SFTVEKGIYQTSNFR | 77 | EILPVSMTKTSVDCT | 182 | LDSFKEELDKYFKNH | 287 |
| EKGIYQTSNFRVQPT | 78 | VSMTKTSVDCTMYIC | 183 | KEELDKYFKNHTSPD | 288 |
| YQTSNFRVQPTESIV | 79 | KTSVDCTMYICGDST | 184 | DKYFKNHTSPDVDLG | 289 |
| NFRVQPTESIVRFPN | 80 | DCTMYICGDSTECSN | 185 | KNHTSPDVDLGDISG | 290 |
| QPTESIVRFPNITNL | 81 | YICGDSTECSNLLLQ | 186 | SPDVDLGDISGINAS | 291 |
| SIVRFPNITNLCPFG | 82 | DSTECSNLLLQYGSF | 187 | DLGDISGINASVVNI | 292 |
| FPNITNLCPFGEVEN | 83 | CSNLLLQYGSFCTQL | 188 | ISGINASVVNIQKEI | 293 |
| TNLCPFGEVFNATRF | 84 | LLQYGSFCTQLNRAL | 189 | NASVVNIQKEIDRLN | 294 |
| PFGEVFNATRFASVY | 85 | GSFCTQLNRALTGIA | 190 | VNIQKEIDRLNEVAK | 295 |
| VFNATRFASVYAWNR | 86 | TQLNRALTGIAVEQD | 191 | KEIDRLNEVAKNLNE | 296 |
| TRFASVYAWNRKRIS | 87 | RALTGIAVEQDKNTQ | 192 | RLNEVAKNLNESLID | 297 |
| SVYAWNRKRISNCVA | 88 | GIAVEQDKNTQEVFA | 193 | VAKNLNESLIDLQEL | 298 |
| WNRKRISNCVADYSV | 89 | EQDKNTQEVFAQVKQ | 194 | LNESLIDLQELGKYE | 299 |
| RISNCVADYSVLYNS | 90 | NTQEVFAQVKQIYKT | 195 | LIDLQELGKYEQYIK | 300 |
| CVADYSVLYNSASFS | 91 | VFAQVKQIYKTPPIK | 196 | QELGKYEQYIKWPWY | 301 |
| YSVLYNSASFSTFKC | 92 | VKQIYKTPPIKDFGG | 197 | KYEQYIKWPWYIWLG | 302 |
| YNSASFSTFKCYGVS | 93 | YKTPPIKDFGGFNFS | 198 | YIKWPWYIWLGFIAG | 303 |
| SFSTFKCYGVSPTKL | 94 | PIKDFGGFNFSQILP | 199 | PWYIWLGFIAGLIAI | 304 |
| FKCYGVSPTKLNDLC | 95 | FGGFNFSQILPDPSK | 200 | WLGFIAGLIAIVMVT | 305 |
| GVSPTKLNDLCFTNV | 96 | NFSQILPDPSKPSKR | 201 | IAGLIAIVMVTIMLC | 306 |
| TKLNDLCFTNVYADS | 97 | ILPDPSKPSKRSFIE | 202 | IAIVMVTIMLCCMTS | 307 |
| DLCFTNVYADSFVIR | 98 | PSKPSKRSFIEDLLF | 203 | MVTIMLCCMTSCCSC | 308 |
| TNVYADSFVIRGDEV | 99 | SKRSFIEDLLFNKVT | 204 | MLCCMTSCCSCLKGC | 309 |
| ADSFVIRGDEVRQIA | 100 | FIEDLLFNKVTLADA | 205 | MTSCCSCLKGCCSCG | 310 |
| VIRGDEVRQIAPGQT | 101 | LLFNKVTLADAGFIK | 206 | CSCLKGCCSCGSCCK | 311 |
| DEVRQIAPGQTGKIA | 102 | KVTLADAGFIKQYGD | 207 | KGCCSCGSCCKFDED | 312 |
| QIAPGQTGKIADYNY | 103 | ADAGFIKQYGDCLGD | 208 | SCGSCCKFDEDDSEP | 313 |
| GQTGKIADYNYKLPD | 104 | FIKQYGDCLGDIAAR | 209 | CCKFDEDDSEPVLKG | 314 |
| KIADYNYKLPDDFTG | 105 | YGDCLGDIAARDLIC | 210 | DEDDSEPVLKGVKLH | 315 |

Overlapping Peptide Sequences Derived from SARS-CoV-2 Membrane Protein [QHD43419.1]

| | SEQ ID | | SEQ ID | | SEQ ID |
|---|---|---|---|---|---|
| Fragment | NO: | Fragment | NO: | Fragment | NO: |
| MADSNGTITVEELK | 316 | INWITGGIAIAMACL | 334 | LRGHLRIAGHHLGR | 352 |
| K | | | | C | |
| NGTITVEELKKLLEQ | 317 | TGGIAIAMACLVGLM | 335 | LRIAGHHLGRCDIKD | 353 |
| TVEELKKLLEQWNL | 318 | AIAMACLVGLMWLS | 336 | GHHLGRCDIKDLPKE | 354 |
| V | | Y | | | |
| LKKLLEQWNLVIGF | 319 | ACLVGLMWLSYFIAS | 337 | GRCDIKDLPKEITVA | 355 |
| L | | | | | |
| LEQWNLVIGFLFLT | 320 | GLMWLSYFIASFRLF | 338 | IKDLPKEITVATSRT | 356 |
| W | | | | | |
| NLVIGFLFLTWICLL | 321 | LSYFIASFRLFARTR | 339 | PKEITVATSRTLSYY | 357 |
| GFLFLTWICLLQFA | 322 | IASFRLFARTRSMW | 340 | TVATSRTLSYYKLGA | 358 |
| Y | | S | | | |
| LTWICLLQFAYANR | 323 | RLFARTRSMWSFNP | 341 | SRTLSYYKLGASQR | 359 |
| N | | E | | V | |
| CLLQFAYANRNRFL | 324 | RTRSMWSFNPETNI | 342 | SYYKLGASQRVAGD | 360 |
| Y | | L | | S | |
| FAYANRNRFLYIIKL | 325 | MWSFNPETNILLNVP | 343 | LGASQRVAGDSGFA | 361 |
| | | | | A | |
| NRNRFLYIIKLIFLW | 326 | NPETNILLNVPLHGT | 344 | QRVAGDSGFAAYSR | 362 |
| | | | | Y | |
| FLYIIKLIFLWLLWP | 327 | NILLNVPLHGTILTR | 345 | GDSGFAAYSRYRIG | 363 |
| | | | | N | |
| IKLIFLWLLWPVTLA | 328 | NVPLHGTILTRPLLE | 346 | FAAYSRYRIGNYKLN | 364 |
| FLWLLWPVTLACFV | 329 | HGTILTRPLLESELV | 347 | SRYRIGNYKLNTDHS | 365 |
| L | | | | | |
| LWPVTLACFVLAAV | 330 | LTRPLLESELVIGAV | 348 | IGNYKLNTDHSSSSD | 366 |
| Y | | | | | |
| TLACFVLAAVYRIN | 331 | LLESELVIGAVILRG | 349 | KLNTDHSSSSDNIAL | 367 |
| W | | | | | |
| FVLAAVYRINWITG | 332 | ELVIGAVILRGHLRI | 350 | TDHSSSSDNIALLVQ | 368 |
| G | | | | | |
| AVYRINWITGGIAIA | 333 | GAVILRGHLRIAGHH | 351 | | |

Overlapping Peptide Sequences Derived from SARS-CoV-2 Nucleoprotein [QHD43423.2]

| | SEQ ID | | SEQ ID | | SEQ ID |
|---|---|---|---|---|---|
| Fragment | NO: | Fragment | NO: | Fragment | NO: |
| MSDNGPQNQRNAPR | 369 | GALNTPKDHIGTRNP | 403 | AFGRRGPEQTQGNF | 437 |
| | | | | G | |
| GPQNQRNAPRITFGG | 370 | TPKDHIGTRNPANNA | 404 | RGPEQTQGNFGDQE | 438 |
| | | | | L | |
| QRNAPRITFGGPSDS | 371 | HIGTRNPANNAAIVL | 405 | QTQGNFGDQELIRQ | 439 |
| | | | | G | |
| PRITFGGPSDSTGSN | 372 | RNPANNAAIVLQLPQ | 406 | NFGDQELIRQGTDYK | 440 |
| FGGPSDSTGSNQNG | 373 | NNAAIVLQLPQGTTL | 407 | QELIRQGTDYKHWP | 441 |
| E | | | | Q | |
| SDSTGSNQNGERSG | 374 | IVLQLPQGTTLPKGF | 408 | RQGTDYKHWPQIAQ | 442 |
| A | | | | F | |
| GSNQNGERSGARSK | 375 | LPQGTTLPKGFYAEG | 409 | DYKHWPQIAQFAPSA | 443 |
| Q | | | | | |
| NGERSGARSKQRRP | 376 | TTLPKGFYAEGSRGG | 410 | WPQIAQFAPSASAFF | 444 |
| Q | | | | | |
| SGARSKQRRPQGLP | 377 | KGFYAEGSRGGSQA | 411 | AQFAPSASAFFGMS | 445 |
| N | | S | | R | |
| SKQRRPQGLPNNTA | 378 | AEGSRGGSQASSRS | 412 | PSASAFFGMSRIGME | 446 |
| S | | S | | | |
| RPQGLPNNTASWFT | 379 | RGGSQASSRSSSRS | 413 | AFFGMSRIGMEVTPS | 447 |
| A | | R | | | |
| LPNNTASWFTALTQH | 380 | QASSRSSSRSRNSSR | 414 | MSRIGMEVTPSGTW | 448 |
| | | | | L | |
| TASWFTALTQHGKED | 381 | RSSSRSRNSSRNSTP | 415 | GMEVTPSGTWLTYT | 449 |
| | | | | G | |
| FTALTQHGKEDLKFP | 382 | RSRNSSRNSTPGSSR | 416 | TPSGTWLTYTGAIKL | 450 |
| TQHGKEDLKFPRGQ | 383 | SSRNSTPGSSRGTSP | 417 | TWLTYTGAIKLDDKD | 451 |
| G | | | | | |
| KEDLKFPRGQGVPIN | 384 | STPGSSRGTSPARMA | 418 | YTGAIKLDDKDPNFK | 452 |
| KFPRGQGVPINTNSS | 385 | SSRGTSPARMAGNG | 419 | IKLDDKDPNFKDQVI | 453 |
| | | G | | | |
| GQGVPINTNSSPDDQ | 386 | TSPARMAGNGGDAAL | 420 | DKDPNFKDQVILLNK | 454 |
| PINTNSSPDDQIGYY | 387 | RMAGNGGDAALALLL | 421 | NFKDQVILLNKHIDA | 455 |
| NSSPDDQIGYYRRAT | 388 | NGGDAALALLLLDRL | 422 | QVILLNKHIDAYKTF | 456 |
| DDQIGYYRRATRRIR | 389 | AALALLLLDRLNQLE | 423 | LNKHIDAYKTFPPTE | 457 |
| GYYRRATRRIRGGDG | 390 | LLLLDRLNQLESKMS | 424 | IDAYKTFPPTEPKKD | 458 |
| RATRRIRGGDGKMK | 391 | DRLNQLESKMSGKG | 425 | KTFPPTEPKKDKKKK | 459 |
| D | | Q | | | |
| RIRGGDGKMKDLSPR | 392 | QLESKMSGKGQQQQ | 426 | PTEPKKDKKKKADET | 460 |
| | | G | | | |
| GDGKMKDLSPRWYF | 393 | KMSGKGQQQQGQTV | 427 | KKDKKKKADETQALP | 461 |
| Y | | T | | | |
| MKDLSPRWYFYYLGT | 394 | KGQQQQGQTVTKKS | 428 | KKKADETQALPQRQ | 462 |
| | | A | | K | |
| SPRWYFYYLGTGPEA | 395 | QQGQTVTKKSAAEAS | 429 | DETQALPQRQKKQQ | 463 |
| | | | | T | |
| YFYYLGTGPEAGLPY | 396 | TVTKKSAAEASKKPR | 430 | ALPQRQKKQQTVTLL | 464 |
| LGTGPEAGLPYGANK | 397 | KSAAEASKKPRQKRT | 431 | RQKKQQTVTLLPAAD | 465 |
| PEAGLPYGANKDGII | 398 | EASKKPRQKRTATKA | 432 | QQTVTLLPAADLDDF | 466 |
| LPYGANKDGIIWVAT | 399 | KPRQKRTATKAYNVT | 433 | TLLPAADLDDFSKQL | 467 |
| ANKDGIIWVATEGAL | 400 | KRTATKAYNVTQAFG | 434 | AADLDDFSKQLQQS | 468 |
| | | | | M | |
| GIIWVATEGALNTPK | 401 | TKAYNVTQAFGRRGP | 435 | DDFSKQLQQSMSSA | 469 |
| | | | | D | |
| VATEGALNTPKDHIG | 402 | NVTQAFGRRGPEQT | 436 | KQLQQSMSSADSTQ | 470 |
| | | Q | | A | |

Overlapping peptide sequences derived from SARS-CoV-2 envelope protein [YP_009724392.1]

| | SEQ ID |
|---|---|
| Fragment | NO: |
| MYSFVSEETGTLIVN | 471 |
| VSEETGTLIVNSVLL | 472 |
| TGTLIVNSVLLFLAF | 473 |
| IVNSVLLFLAFVVFL | 474 |
| VLLFLAFVVFLLVTL | 475 |
| LAFWVFLLVTLAILT | 476 |
| VFLLVTLAILTALRL | 477 |
| VTLAILTALRLCAYC | 478 |
| ILTALRLCAYCCNIV | 479 |
| LRLCAYCCNIVNVSL | 480 |
| AYCCNIVNVSLVKPS | 481 |
| NIVNVSLVKPSFYVY | 482 |
| VSLVKPSFYVYSRVK | 483 |
| KPSFYVYSRVKNLNS | 484 |
| YVYSRVKNLNSSRVP | 485 |
| RVKNLNSSRVPDLLV | 486 |

### APPENDIX 3—PEPTIDES SEQUENCES WITH IDENTIFIED HOMOLOGY TO ENDEMIC HUMAN CORONAVIRUSES

### a) High Homology Cut Off

| Spike | Membrane | Nucleoprotein |
|---|---|---|
| PSKPSKRSFIEDLLF | FLYIIKLIFLWLLWP | GDGKMKDLSPRWYFY |
| SKRSFIEDLLFNKVT | RLFARTRSMWSFNPE | MKDLSPRWYFYYLGT |
| FIEDLLFNKVTLADA | RTRSMWSFNPETNIL | SPRWYFYYLGTGPEA |
| LICAQKFNGLTVLPP | | YFYYLGTGPEAGLPY |
| IGVTQNVLYENQKLI | | KPRQKRTATKAYNVT |
| QNVLYENQKLIANQF | | |
| YENQKLIANQFNSAI | | |
| TASALGKLQDVVNQN | | |
| LGKLQDVVNQNAQAL | | |
| QDVVNQNAQALNTLV | | |
| NQNAQALNTLVKQLS | | |
| NFGAISSVLNDILSR | | |
| LSRLDKVEAEVQIDR | | |
| DKVEAEVQIDRLITG | | |
| AEVQIDRLITGRLQS | | |
| IDRLITGRLQSLQTY | | |
| KEELDKYFKNHTSPD | | |
| KYEQYIKWPWYIWLG | | |

### b) Homology Detected (No Cut Off)

| Spike | Membrane | Nucleoprotein |
|---|---|---|
| TDAVRDPQTLEILDI | NRNRFLYIIKLIFLW | GDGKMKDLSPRWYFY |
| RDPQTLEILDITPCS | FLYIIKLIFLWLLWP | MKDLSPRWYFYYLGT |
| AIPTNFTISVTTEIL | IKLIFLWLLWPVTLA | SPRWYFYYLGTGPEA |
| ILPDPSKPSKRSFIE | GLMWLSYFIASFRLF | YFYYLGTGPEAGLPY |
| PSKPSKRSFIEDLLF | LSYFIASFRLFARTR | KPRQKRTATKAYNVT |
| SKRSFIEDLLFNKVT | IASFRLFARTRSMWS | |
| FIEDLLFNKVTLADA | RLFARTRSMWSFNPE | |
| LLFNKVTLADAGFIK | RTRSMWSFNPETNIL | |
| AARDLICAQKFNGLT | MWSFNPETNILLNVP | |
| LICAQKFNGLTVLPP | | |
| QKFNGLTVLPPLLTD | | |
| GLTVLPPLLTDEMIA | | |
| IGVTQNVLYENQKLI | | |
| QNVLYENQKLIANQF | | |
| YENQKLIANQFNSAI | | |
| TASALGKLQDVVNQN | | |
| LGKLQDVVNQNAQAL | | |
| QDVVNQNAQALNTLV | | |
| MTSCCSCLKGCCSCG | | |

### Comparative Example 1—MHC Binding Predictions

In an alternative approach to panel construction, performed for illustrative purposes only, a list of predicted MHC binding epitopes were generated by using the TepiTool software from the internet Epitope Database (IEDB.org). Predicted MHC class I and class II-binding peptides were predicted from the spike protein for the 27 most common HLA class I allelles and the 26 most common HLA class II alleles (appendix 4 for raw TepiTool results). Once duplicate peptides were removed, a list of 117 9mers and 137 15mers were generated spanning the spike, envelope and nucleocapsid proteins (appendix 4a).

This list was then examined for homology using the BLAST search tool as described above. 29 peptides were identified as having high homology (>=9aa matches) to human coronaviruses (appendix 4b), and 90 peptides (appendix 4c) had homology when the lower homology criteria was used.

### APPENDIX 4

a) Predicted MHC Class I and Class II Binding Peptides from SARS-CoV-2 Genes

| | Peptide | Peptide | SEQ ID | | Peptide | Peptide | SEQ ID |
|---|---|---|---|---|---|---|---|
| Sequence | start | end | NO: | Sequence | start | end | NO: |
| SPRRARSVA | 680 | 688 | 487 | GNFKNLREFVFKNID | 184 | 198 | 614 |
| LTDEMIAQY | 865 | 873 | 488 | YLQPRTFLLKYNENG | 269 | 283 | 615 |
| YEQYIKWPW | 1206 | 1214 | 489 | PTNFTISVTTEILPV | 715 | 729 | 616 |
| RISNCVADY | 357 | 365 | 490 | VFLHVTYVPAQEKNF | 1061 | 1075 | 617 |
| YNYLYRLFR | 449 | 457 | 491 | SFPQSAPHGVVFLHV | 1051 | 1065 | 618 |
| MTSCCSCLK | 1237 | 1245 | 492 | CTFEYVSQPFLMDLE | 166 | 180 | 619 |
| NSASFSTFK | 370 | 378 | 493 | SVLYNSASFSTFKCY | 366 | 380 | 620 |
| FIAGLIAIV | 1220 | 1228 | 494 | FQFCNDPFLGVYYH | 133 | 147 | 621 |
| | | | | K | | | |
| VYSTGSNVF | 635 | 643 | 495 | CSNLLLQYGSFCTQL | 749 | 763 | 622 |
| ETKCTLKSF | 298 | 306 | 496 | QYIKWPWYIWLGFIA | 1208 | 1222 | 623 |
| NYNYLYRLF | 448 | 456 | 497 | PWYIWLGFIAGLIAI | 1213 | 1227 | 624 |
| YFPLQSYGF | 489 | 497 | 498 | LREFVFKNIDGYFKI | 189 | 203 | 625 |
| VYYPDKVFR | 36 | 44 | 499 | YNYLYRLFRKSNLKP | 449 | 463 | 626 |
| KQGNFKNLR | 182 | 190 | 500 | IKDFGGFNFSQILPD | 794 | 808 | 627 |
| YQDVNCTEV | 612 | 620 | 501 | DLCFTNVYADSFVIR | 389 | 403 | 628 |
| LPFFSNVTW | 56 | 64 | 502 | ESNKKFLPFQQFGR | 554 | 568 | 629 |
| | | | | D | | | |
| TPGDSSSGW | 250 | 258 | 503 | TAGAAAYYVGYLQP | 259 | 273 | 630 |
| | | | | R | | | |
| WPWYIWLGF | 1212 | 1220 | 504 | FNCYFPLQSYGFQPT | 486 | 500 | 631 |
| FTISVTTEI | 718 | 726 | 505 | ENQKLIANQFNSAIG | 918 | 932 | 632 |
| NTQEVFAQV | 777 | 785 | 506 | DEMIAQYTSALLAGT | 867 | 881 | 633 |
| KIYSKHTPI | 202 | 210 | 507 | PSKPSKRSFIEDLLF | 809 | 823 | 634 |
| FAMQMAYRF | 898 | 906 | 508 | AGLIAIVMVTIMLCC | 1222 | 1236 | 635 |
| TTRTQLPPA | 19 | 27 | 509 | NIIRGWIFGTTLDSK | 99 | 113 | 636 |
| ATRFASVYA | 344 | 352 | 510 | KVGGNYNYLYRLFR | 444 | 458 | 637 |
| | | | | K | | | |
| LAIPTNFTI | 712 | 720 | 511 | VYYPDKVFRSSVLHS | 36 | 50 | 638 |
| PYRVVVLSF | 507 | 515 | 512 | GTGVLTESNKKFLPF | 548 | 562 | 639 |
| AENSVAYSN | 701 | 709 | 513 | NDGVYFASTEKSNII | 87 | 101 | 640 |
| VLNDILSRL | 976 | 984 | 514 | TRFQTLLALHRSYLT | 236 | 250 | 641 |
| GTHWFVTQR | 1099 | 1107 | 515 | RLFRKSNLKPFERDI | 454 | 468 | 642 |
| KSWMESEFR | 150 | 158 | 516 | LDSFKEELDKYFKNH | 1145 | 1159 | 643 |
| QIYKTPPIK | 787 | 795 | 517 | LQSLQTYVTQQLIRA | 1001 | 1015 | 644 |
| VLPFNDGVY | 83 | 91 | 518 | FGAISSVLNDILSRL | 970 | 984 | 645 |
| LAGTITSGW | 878 | 886 | 519 | QKFNGLTVLPPLLTD | 853 | 867 | 646 |
| YLQPRTFLL | 269 | 277 | 520 | FVTQRNFYEPQIITT | 1103 | 1117 | 647 |
| YTNSFTRGV | 28 | 36 | 521 | IKVCEFQFCNDPFLG | 128 | 142 | 648 |
| KQIYKTPPI | 786 | 794 | 522 | EHVNNSYECDIPIGA | 654 | 668 | 649 |
| LGAENSVAY | 699 | 707 | 523 | CNGVEGFNCYFPLQ | 480 | 494 | 650 |
| | | | | S | | | |
| ASFSTFKCY | 372 | 380 | 524 | DPLQPELDSFKEELD | 1139 | 1153 | 651 |
| SSTASALGK | 939 | 947 | 525 | AAEIRASANLAATKM | 1015 | 1029 | 652 |
| QELGKYEQY | 1201 | 1209 | 526 | SLLIVNNATNVVIKV | 116 | 130 | 653 |
| IYQTSNFRV | 312 | 320 | 527 | TQLNRALTGIAVEQD | 761 | 775 | 654 |
| FLHVTYVPA | 1062 | 1070 | 528 | TNTSNQVAVLYQDV | 602 | 616 | 655 |
| | | | | N | | | |
| SVYAWNRKR | 349 | 357 | 529 | ASANLAATKMSECVL | 1020 | 1034 | 656 |
| NASVVNIQK | 1173 | 1181 | 530 | FGAGAALQIPFAMQ | 888 | 902 | 657 |
| | | | | M | | | |
| EVFNATRFA | 340 | 348 | 531 | QYTSALLAGTITSGW | 872 | 886 | 658 |
| FSTFKCYGV | 374 | 382 | 532 | TYVTQQLIRAAEIRA | 1006 | 1020 | 659 |
| RFDNPVLPF | 78 | 86 | 533 | TWRVYSTGSNVFQT | 632 | 646 | 660 |
| | | | | R | | | |
| KSFTVEKGI | 304 | 312 | 534 | GDISGINASVVNIQK | 1167 | 1181 | 661 |
| FPQSAPHGV | 1052 | 1060 | 535 | FNFNGLTGTGVLTES | 541 | 555 | 662 |
| VGGNYNYLY | 445 | 453 | 536 | EDLLFNKVTLADAGF | 819 | 833 | 663 |
| YYVGYLQPR | 265 | 273 | 537 | DSSSGWTAGAAAYY | 253 | 267 | 664 |
| | | | | V | | | |
| TNSFTRGVY | 29 | 37 | 538 | VVNQNAQALNTLVK | 951 | 965 | 665 |
| | | | | Q | | | |
| TLADAGFIK | 827 | 835 | 539 | AKNLNESLIDLQELG | 1190 | 1204 | 666 |
| WFLHVTYV | 1060 | 1068 | 540 | LDKVEAEVQIDRLIT | 984 | 998 | 667 |
| LPFNDGVYF | 84 | 92 | 541 | ITSGWTFGAGAALQI | 882 | 896 | 668 |
| NSFTRGVYY | 30 | 38 | 542 | DLPQGFSALEPLVDL | 215 | 229 | 669 |
| LVKQLSSNF | 962 | 970 | 543 | ALTGIAVEQDKNTQE | 766 | 780 | 670 |
| ITPCSFGGV | 587 | 595 | 544 | INASVVNIQKEIDRL | 1172 | 1186 | 671 |
| KIADYNYKL | 417 | 425 | 545 | NCTEVPVAIHADQLT | 616 | 630 | 672 |
| RARSVASQS | 683 | 691 | 546 | NVYADSFVIRGDEVR | 394 | 408 | 673 |
| LPDDFTGCV | 425 | 433 | 547 | PVAIHADQLTPTWRV | 621 | 635 | 674 |
| PFAMQMAYR | 897 | 905 | 548 | DIPIGAGICASYQTQ | 663 | 677 | 675 |
| ITDAVDCAL | 285 | 293 | 549 | LDITPCSFGGVSVIT | 585 | 599 | 676 |
| GTITSGWTF | 880 | 888 | 550 | CSFGGVSVITPGTNT | 590 | 604 | 677 |
| TLKSFTVEK | 302 | 310 | 551 | VKQLSSNFGAISSVL | 963 | 977 | 678 |
| QTNSPRRAR | 677 | 685 | 552 | NPVLPFNDGVYFAST | 81 | 95 | 679 |
| RQIAPGQTG | 408 | 416 | 553 | SFELLHAPATVCGPK | 514 | 528 | 680 |
| FVSNGTHWF | 1095 | 1103 | 554 | QIPFAMQMAYRENGI | 895 | 909 | 681 |
| LPPAYTNSF | 24 | 32 | 555 | LTVLPPLLTDEMIAQ | 858 | 872 | 682 |
| LPPLLTDEM | 861 | 869 | 556 | AEVQIDRLITGRLQS | 989 | 1003 | 683 |
| HLMSFPQSA | 1048 | 1056 | 557 | DGYFKIYSKHTPINL | 198 | 212 | 684 |
| SKRVDFCGK | 1037 | 1045 | 558 | INLVRDLPQGFSALE | 210 | 224 | 685 |
| FQTRAGCLI | 643 | 651 | 559 | SFVIRGDEVRQIAPG | 399 | 413 | 686 |
| GWTAGAAAY | 257 | 265 | 560 | ISNCVADYSVLYNSA | 358 | 372 | 687 |
| KCYGVSPTK | 378 | 386 | 561 | FYEPQIITTDNTFVS | 1109 | 1123 | 688 |
| SVLNDILSR | 975 | 983 | 562 | IITTDNTFVSGNCDV | 1114 | 1128 | 689 |
| ENGTITDAV | 281 | 289 | 563 | KVFRSSVLHSTQDLF | 41 | 55 | 690 |
| YRLFRKSNL | 453 | 461 | 564 | APAICHDGKAHFPRE | 1078 | 1092 | 691 |
| IPTNFTISV | 714 | 722 | 565 | SFTRGVYYPDKVFRS | 31 | 45 | 692 |
| DVNCTEVPV | 614 | 622 | 566 | SVLNDILSRLDKVEA | 975 | 989 | 693 |
| ITSGWTFGA | 882 | 890 | 567 | GVTQNVLYENQKLIA | 910 | 924 | 694 |
| NATRFASVY | 343 | 351 | 568 | VSQPFLMDLEGKQG | 171 | 185 | 695 |
| | | | | N | | | |
| LIAIVMVTI | 1224 | 1232 | 569 | GFNFSQILPDPSKPS | 799 | 813 | 696 |
| STECSNLLL | 746 | 754 | 570 | LQYGSFCTQLNRALT | 754 | 768 | 697 |
| QIAPGQTGK | 409 | 417 | 571 | QTSNFRVQPTESIVR | 314 | 328 | 698 |
| EILPVSMTK | 725 | 733 | 572 | DPFLGVYYHKNNKS | 138 | 152 | 699 |
| | | | | W | | | |
| GQTGKIADY | 413 | 421 | 573 | EGVFVSNGTHWFVT | 1092 | 1106 | 700 |
| | | | | Q | | | |
| FPNITNLCP | 329 | 337 | 574 | IQDSLSSTASALGKL | 934 | 948 | 701 |
| FIKQYGDCL | 833 | 841 | 575 | WFHAIHVSGTNGTK | 64 | 78 | 702 |
| | | | | R | | | |
| LITGRLQSL | 996 | 1004 | 576 | AGICASYQTQTNSPR | 668 | 682 | 703 |
| TAGAAAYYV | 259 | 267 | 577 | GNCDWVIGIVNNTVY | 1124 | 1138 | 704 |
| YGFQPTNGV | 495 | 503 | 578 | KPFERDISTEIYQAG | 462 | 476 | 705 |
| KNFTTAPAI | 1073 | 1081 | 579 | QPTESIVRFPNITNL | 321 | 335 | 706 |
| FIEDLLFNK | 817 | 825 | 580 | NGTHWFVTQRNFYE | 1098 | 1112 | 707 |
| | | | | P | | | |
| VYADSFVIR | 395 | 403 | 581 | MQMAYRFNGIGVTQ | 900 | 914 | 708 |
| | | | | N | | | |
| GVLTESNKK | 550 | 558 | 582 | RFNGIGVTQNVLYEN | 905 | 919 | 709 |
| STEKSNIIR | 94 | 102 | 583 | EELDKYFKNHTSPDV | 1150 | 1164 | 710 |
| YNSASFSTF | 369 | 377 | 584 | SWMESEFRVYSSAN | 151 | 165 | 711 |
| | | | | N | | | |
| VLSFELLHA | 512 | 520 | 585 | FSNVTWFHAIHVSGT | 59 | 73 | 712 |
| FTNVYADSF | 392 | 400 | 586 | GTTLDSKTQSLLIVN | 107 | 121 | 713 |
| DEDDSEPVL | 1257 | 1265 | 587 | PRRARSVASQSIIAY | 681 | 695 | 714 |
| DCLGDIAAR | 839 | 847 | 588 | STGSNVFQTRAGCLI | 637 | 651 | 715 |
| LEILDITPC | 582 | 590 | 589 | LLALHRSYLTPGDSS | 241 | 255 | 716 |
| AYSNNSIAI | 706 | 714 | 590 | AQALNTLVKQLSSNF | 956 | 970 | 717 |
| RLDKVEAEV | 983 | 991 | 591 | SQSIIAYTMSLGAEN | 689 | 703 | 718 |
| NLCPFGEVF | 334 | 342 | 592 | FRVYSSANNCTFEYV | 157 | 171 | 719 |
| FQPTNGVGY | 497 | 505 | 593 | TRFASVYAWNRKRIS | 345 | 359 | 720 |
| FVSGNCDVV | 1121 | 1129 | 594 | VYAWNRKRISNCVA | 350 | 364 | 721 |
| | | | | D | | | |
| PWYIWLGFI | 1213 | 1221 | 595 | CGKGYHLMSFPQSA | 1043 | 1057 | 722 |
| | | | | P | | | |
| RAAEIRASA | 1014 | 1022 | 596 | DDSEPVLKGVKLHYT | 1259 | 1273 | 723 |
| KLNDLCFTN | 386 | 394 | 597 | DRLITGRLQSLQTYV | 994 | 1008 | 724 |
| ASVYAWNRK | 348 | 356 | 598 | TFLLKYNENGTITDA | 274 | 288 | 725 |
| LEPLVDLPI | 223 | 231 | 599 | GKLQDVVNQNAQAL | 946 | 960 | 726 |
| | | | | N | | | |
| SLSSTASAL | 937 | 945 | 600 | AENSVAYSNNSIAIP | 701 | 715 | 727 |
| FPLQSYGFQ | 490 | 498 | 601 | RLNEVAKNLNESLID | 1185 | 1199 | 728 |
| NIDGYFKIY | 196 | 204 | 602 | STNLVKNKCVNFNFN | 530 | 544 | 729 |
| QTYVTQQLI | 1005 | 1013 | 603 | CVIAWNSNNLDSKV | 432 | 446 | 730 |
| | | | | G | | | |
| GYQPYRVVVLSFEL | 504 | 518 | 604 | LVDLPIGINITRFQT | 226 | 240 | 731 |
| L | | | | | | | |
| RVVVLSFELLHAPAT | 509 | 523 | 605 | SMTKTSVDCTMYICG | 730 | 744 | 732 |
| EVFNATRFASVYAW | 340 | 354 | 606 | QFGRDIADTTDAVRD | 564 | 578 | 733 |
| N | | | | | | | |
| IGINITRFQTLLALH | 231 | 245 | 607 | EKGIYQTSNFRVQPT | 309 | 323 | 734 |
| MFVFLVLLPLVSSQC | 1 | 15 | 608 | AYTMSLGAENSVAY | 694 | 708 | 735 |
| | | | | S | | | |
| LHSTQDLFLPFFSNV | 48 | 62 | 609 | KNKCVNFNFNGLTG | 535 | 549 | 736 |
| | | | | T | | | |
| KRSFIEDLLFNKVTL | 814 | 828 | 610 | LLPLVSSQCVNLTTR | 7 | 21 | 737 |
| LFLPFFSNVTWFHAI | 54 | 68 | 611 | EVFAQVKQIYKTPPI | 780 | 794 | 738 |
| APHGVVFLHVTYVP | 1056 | 1070 | 612 | KNFTTAPAICHDGKA | 1073 | 1087 | 739 |
| A | | | | | | | |
| AYYVGYLQPRTFLLK | 264 | 278 | 613 | LCPFGEVFNATRFAS | 335 | 349 | 740 |

b) MHC Binding Peptides with High Homology to Endemic Human Coronaviruses

| | | |
|---|---|---|
| FIAGLIAIV | PSKPSKRSFIEDLLF | QIPFAMQMAYRENGI |
| IAIPTNFTI | LDSFKEELDKYFKNH | AEVQIDRLITGRLQS |
| FIEDLLFNK | FGAISSVLNDILSRL | SVLNDILSRLDKVEA |
| KRSFIEDLLFNKVTL | QKFNGLTVLPPLLTD | LQYGSFCTQLNRALT |
| APHGVVFLHVTYVPA | DPLQPELDSFKEELD | EELDKYFKNHTSPDV |
| PTNFTISVTTEILPV | TYVTQQLIRAAEIRA | CGKGYHLMSFPQSAP |
| CSNLLLQYGSFCTQL | EDLLFNKVTLADAGF | DRLITGRLQSLQTYV |
| QYIKWPWYIWLGFIA | VVNQNAQALNTLVKQ | GKLQDVVNQNAQALN |
| PWYIWLGFIAGLIAI | LDKVEAEVQIDRLIT | RLNEVAKNLNESLID |
| ENQKLIANQFNSAIG | VKQLSSNFGAISSVL | |

c) MHC Binding Peptides with Homology to Endemic Human Coronaviruses

| | | |
|---|---|---|
| YEQYIKWPW | PSKPSKRSFIEDLLF | FYEPQIITTDNTFVS |
| WPWYIWLGF | NIIRGWIFGTTLDSK | IITTDNTFVSGNCDV |
| LAIPTNFTI | GTGVLTESNKKFLPF | KVFRSSVLHSTQDLF |
| EVFNATRFA | TRFQTLLALHRSYLT | SVLNDILSRLDKVEA |
| FVSNGTHWF | LDSFKEELDKYFKNH | GVTQNVLYENQKLIA |
| LITGRLQSL | FGAISSVLNDILSRL | GFNFSQILPDPSKPS |
| FIEDLLFNK | QKFNGLTVLPPLLTD | LQYGSFCTQLNRALT |
| GYQPYRVWLSFELL | EHVNNSYECDIPIGA | DPFLGVYYHKNNKSW |
| EVFNATRFASVYAWN | CNGVEGFNCYFPLQS | EGVFVSNGTHWFVTQ |
| IGINITRFQTLLALH | DPLQPELDSFKEELD | IQDSLSSTASALGKL |
| MFVFLVLLPLVSSQC | AAEIRASANLAATKM | KPFERDISTEIYQAG |
| LHSTQDLFLPFFSNV | TNTSNQVAVLYQDVN | NGTHWFVTQRNFYEP |
| KRSFIEDLLFNKVTL | ASANLAATKMSECVL | MQMAYRFNGIGVTQN |
| LFLPFFSNVTWFHAI | QYTSALLAGTITSGW | RFNGIGVTQNVLYEN |
| APHGVVFLHVTYVPA | TYVTQQLIRAAEIRA | EELDKYFKNHTSPDV |
| AYYVGYLQPRTFLLK | FNFNGLTGTGVLTES | GTTLDSKTQSLLIVN |
| GNFKNLREFVFKNID | EDLLFNKVTLADAGF | LLALHRSYLTPGDSS |
| YLQPRTFLLKYNENG | DSSSGWTAGAAAYYV | AQALNTLVKQLSSNF |
| PTNFTISVTTEILPV | WVNQNAQALNTLVKQ | SQSIIAYTMSLGAEN |
| VFLHVTYVPAQEKNF | LDKVEAEVQIDRLIT | CGKGYHLMSFPQSAP |
| SFPQSAPHGVVFLHV | ITSGWTFGAGAALQI | DRLITGRLQSLQTYV |
| CTFEYVSQPFLMDLE | DLPQGFSALEPLVDL | TFLLKYNENGTITDA |
| SVLYNSASFSTFKCY | INASVVNIQKEIDRL | GKLQDVVNQNAQALN |
| CSNLLLQYGSFCTQL | NVYADSFVIRGDEVR | RLNEVAKNLNESLID |
| QYIKWPWYIWLGFIA | LDITPCSFGGVSVIT | STNLVKNKCVNFNFN |
| PWYIWLGFIAGLIAI | CSFGGVSVITPGTNT | LVDLPIGINITRFQT |
| YNYLYRLFRKSNLKP | VKQLSSNFGAISSVL | SMTKTSVDCTMYICG |
| IKDFGGFNFSQILPD | NPVLPFNDGVYFAST | AYTMSLGAENSVAYS |
| FNCYFPLQSYGFQPT | QIPFAMQMAYRENGI | KNKCVNFNFNGLTGT |
| ENQKLIANQFNSAIG | AEVQIDRLITGRLQS | LCPFGEVFNATRFAS |

### Example 2—Use of Optimised Pools of Fragments Derived from SARS-CoV-2 Proteins

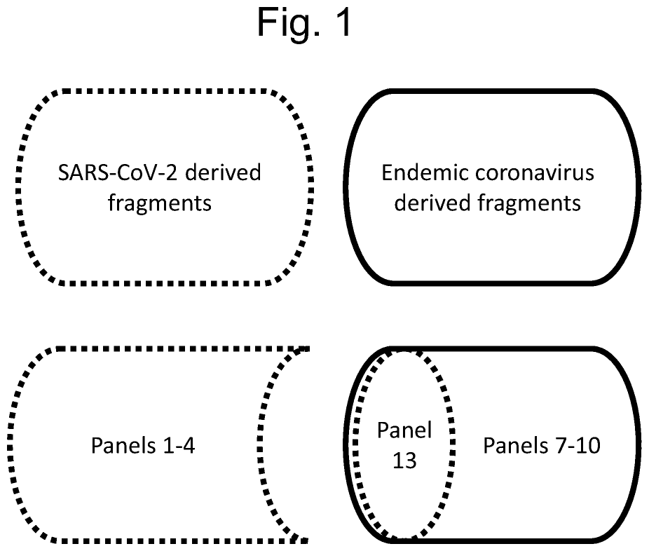

ELISpot assays were performed using PBMC samples obtained from healthy donors. Various fragment pools were separately contacted with the PBMC samples in order to perform the ELISpot:
“P1-4” comprising panel 1, 2, 3 or 4 respectively. Each of panels 1 to 4 is a fragment pool in which the fragments form a protein fragment library encompassing the sequence of a SARS-CoV-2 protein. The fragments are 15 amino acids in length and overlap by 11 amino acids. Fragments having a homolog that is an amino acid sequence that has at least 60% sequence identity to the fragment and is expressed by one or more of the endemic common cold coronaviruses are excluded from the protein fragment library. For panel 1, the SARS-CoV-2 protein is SARS-CoV-2 S1 spike domain (S1). For panel 2, the SARS-CoV-2 protein is SARS-CoV-2 S2 spike domain (S2). For panel 3, the SARS-CoV-2 protein is SARS-CoV-2 nucleocapsid protein (N). For panel 4, the SARS-CoV-2 protein is SARS-CoV-2 membrane protein (M).
“P13” comprising the fragments excluded from P1-4. The fragments in P13 each have a homolog that is an amino acid sequence that has at least 60% sequence identity to the fragment and is expressed by one or more of the endemic common cold coronaviruses (HKU1, OC43, 229E and NL63). The fragments comprised in P13 are set out in Table 3 below.
“P7-10” comprising one of panel 7, 8, 9 or 10 respectively. Each of panels 7 to 10 is a fragment pool in which the fragments form a protein fragment library encompassing the sequence of spike glycoprotein from a different endemic human coronavirus (P7=HKU1, P8=229E, P9=NL63, P10=OC43). The fragments are 15 amino acids in length and overlap by 11 amino acids.
   P1-4, P13 and P7-10 are represented graphically in FIG. 1.

TABLE 3
fragments comprised in panel 13 (P13)

| ProtEin | |
|---|---|
| S1 | TDAVRDPQTLEILDI |
| S1 | RDPQTLEILDITPCS |
| S2 | PSKPSKRSFIEDLLF |
| S2 | SKRSFIEDLLFNKVT |
| S2 | FIEDLLENKVTLADA |
| S2 | LICAQKFNGLTVLPP |
| S2 | IGVTQNVLYENQKLI |
| S2 | QNVLYENQKLIANOF |
| S2 | YENQKLIÅNGFNSAI |
| S2 | TASALGKLQDVVNQN |
| S2 | LGKLQDVVNQNADAL |
| S2 | QDVVNQNAQALNTIV |
| S2 | NQNAQALNTLVKQLS |
| S2 | NFGAISSVLNDILSR |
| S2 | LSRLDKVEAEVQIDR |
| S2 | DKVEAEVQIDRLITG |
| S2 | AEVQIDRLITGRLQS |
| S2 | IDRLITGRLQSLQTY |
| S2 | KEELDKYFKNHTSPD |
| S2 | KYEQYIKWPWYIWLG |
| N | GDGKMKDLSPRWYFY |
| N | MKDLSPRWYFYYLGT |
| N | SPRWYFYYLGTGPEA |
| N | YFYYLGTGPEAGLPY |
| N | KPRQKRTATKAYNVT |
| M | FLYIIKLIFLWLLWP |
| M | RTRSMWSFNPETNIL |
| M | MWSFNPETNILLNVP |

### Results

12% (53/449) were reactive to one of P1, P3 and P4.
76% (219/289) responded to Spike from at least one of the endemic strains, P7-10.
10% (47/449) responded to P13. For those subjects responding, the mean adjusted spot count was 16.5 (sd 13.6), the median was 11, and the range was from 6 to 64.
   In order to assess the value of P13 in distinguishing SARS-CoV-2 specific immune responses from cross-reactive immune responses primed by endemic coronaviruses, P13 reactive samples were allocated into the following groups:

| | P13 | P 1-4 | P 7-10 | | |
|---|---|---|---|---|---|
| | reactive | reactive | reactive | N | Interpretation |
| Group 1 | Yes | Yes | Yes | N = 15 | P13 responses cannot be |
| | | | | | attributed to covid19 |
| | | | | | exposure. However |
| | | | | | these cases were picked |
| | | | | | up by P1-4 anyway. |
| | | | | | All subjects in this group |
| | | | | | reactive to P7-10 have |
| | | | | | counts of less than 10 |
| Group 2 | Yes | No | Yes | N = 20 | P13 responses may be |
| | | | | | attributed to prior |
| | | | | | exposure to endemic |
| | | | | | coronaviruses. |
| | | | | | P13 sequences originated |
| | | | | | from covid-19 genome |
| | | | | | therefore exposure to |
| | | | | | covid19 cannot be |
| | | | | | excluded, but the |
| | | | | | presence of reactivity to |
| | | | | | P7-10 (and the fact that |
| | | | | | this is a clean cohort of |
| | | | | | presumed covid-19- |
| | | | | | naïve individuals) points |
| | | | | | to pre-existing non- |
| | | | | | covid19 immunity. |
| Group 3 | Yes | Yes | No | N = 3 | The counts for all these |
| | | | | | subjects for panels 1 to 4 |
| | | | | | range from 7 to 55. |
| | | | | | P13 responses might be |
| | | | | | attributable to covid19 |
| | | | | | exposure. |
| Group 4 | Yes | No | No | N = 6 | |
| Group 5 | Yes | Yes | Not | N = 1 | |
| | | | tested | | |
| Group 6 | Yes | No | Not | N = 2 | |
| | | | tested | | |

Based on this dataset, it seems that in most cases P13 responses could be attributed to a prior exposure to endemic strains of coronaviruses (group 2). When individuals react (i.e. raise a T-cell immune response) to endemic strains, only a small proportion also react to SARS Cov-2 (i.e. Panel 13). Cross-reactivity between CCCs and SARS-CoV-2 is not, therefore, common in the population. However, it is possible that such responses provide some protection against COVID-19. P13 may have utility in screening for pre-existing cross-reactive immune responses for SARS-CoV-2 primed by prior exposure to one or more endemic coronaviruses.

P1-4 are optimised for high specificity for SARS-CoV-2. These pools exclude fragments that are potentially cross-reactive with homologs found in endemic coronaviruses. P1-4 may have utility in screening for SARS-CoV-2 specific immune responses.

Summary of Immune Reactive Responses to SARS Cov-2 Peptide Pools and Spike from CCCs Peptide Pools

| P13 | P1-4 | P 7-10 Reactive | | | |
|---|---|---|---|---|---|
| Reactive | Reactive | Yes | No | N/A | Total |
| Yes | Yes | 15 | 3 | 1 | 19 |
| | No | 20 | 6 | 2 | 28 |
| | Total | 35 | 9 | 3 | 47 |
| No | Yes | 21 | 11 | 7 | 39 |
| | No | 163 | 150 | 50 | 363 |
| | Total | 184 | 61 | 57 | 402 |

1. A method for producing a pool of fragments derived from a microbial protein, comprising:
(a) identifying fragments of the microbial protein that are comprised in a protein fragment library encompassing at least 80% of the sequence of the microbial protein;
(b) determining for each fragment identified in step (a) whether or not a homolog exists, wherein the homolog is an amino acid sequence that has at least 60% sequence identity to the fragment and is expressed by one or more microbes in the same family as the microbe from which the microbial protein is derived; and
(c) preparing a pool of fragments in which:
(i) each fragment is a fragment identified in step (a) for which step (b) determines the existence of a homolog; or
(ii) each fragment is a fragment identified in step (a) for which step (b) does not determine the existence of a homolog, and the fragments form a protein fragment library encompassing at least 80% of the sequence of the microbial protein.

2. A pool of fragments derived from a microbial protein, wherein:
(I) each fragment is comprised in a protein fragment library encompassing at least 80% of the sequence of the microbial protein, and has a homolog that is an amino acid sequence that has at least 60% sequence identity to the fragment and is expressed by one or more microbes in the same family as the microbe from which the microbial protein is derived; or
(II) the fragments form a protein fragment library encompassing at least 80% of the sequence of the microbial protein, and each fragment does not have a homolog that is an amino acid sequence that has at least 60% sequence identity to the fragment and is expressed by one or more microbes in the same family as the microbe from which the microbial protein is derived.

3. The pool of claim 2, produced according to the method of claim 1.

4. The method of claim 1, or the pool of claim 2 or 3, wherein the pool comprises fragments whose sequences overlap, optionally wherein the sequences overlap by 11 amino acids.

5. The method of claim 1 or 4, or the pool of any one of claims 2 to 4, wherein the fragments are 15 amino acids in length.

6. The method of claim 1, 4 or 5, or the pool of any one of claims 2 to 5, wherein the microbe from which the microbial protein is derived is an emerging pathogen.

7. The method of any one of claims 1 and 4 to 6, or the pool of any one of claims 2 to 6, wherein one or more of the microbes expressing the homolog is endemic within a population.

8. The method of any one of claims 1 and 4 to 7, or the pool of any one of claims 2 to 7, wherein the microbe from which the microbial protein is derived and the microbe expressing the homolog are each capable of infecting the same species.

9. The method or pool of claim 8, wherein the species is human.

10. The method of any one of claims 1 and 4 to 9, or the pool of any one of claims 2 to 9, wherein the family is Coronaviridae.

11. The method of any one of claims 1 and 4 to 10, or the pool of any one of claims 2 to 10, wherein the microbe from which the microbial protein is derived is a coronavirus.

12. The method or pool of claim 11, wherein the coronavirus is SARS-CoV-2.

13. The method of any one of claims 1 and 4 to 12, or the pool of any one of claims 2 to 12, wherein one or more of the microbes expressing the homolog is a coronavirus.

14. The method or pool of claim 13, wherein one or more of the microbes expressing the homolog is an endemic human coronavirus.

15. The method or pool of claim 14, wherein one or more of the microbes expressing the homolog is selected from HKU1, OC43, 229E and NL63.

16. The method of any one of claims 1 and 4 to 15, or the pool of any one of claims 2 to 15, wherein the microbial protein is selected from SARS-CoV-2 S1 spike domain, SARS-CoV-2 S2 spike domain, SARS-CoV-2 nucleocapsid protein, SARS-CoV-2 membrane protein, and SARS-CoV-2 envelope protein.

17. A consolidated pool of fragments which comprises two or more pools as defined in any one of claims 2 to 16, wherein each of the two or more pools comprises fragments derived from a different microbial protein, optionally wherein the microbial protein is selected from SARS-CoV-2 S1 spike domain, SARS-CoV-2 S2 spike domain, SARS-CoV-2 nucleocapsid protein, SARS-CoV-2 membrane protein, and SARS-CoV-2 envelope protein.

18. The consolidated pool of claim 17, wherein the pool comprises or consists of the fragments set out in Table 3.

19. A method for determining the presence or absence of immune cells targeting a microbe, the method comprising contacting a sample comprising immune cells with one or more pools as defined in any one of claims 2 to 18, and detecting in vitro the presence or absence of an immune response to the one or more pools.

20. The method of claim 19, wherein the sample is contacted with each of the one or more pools in a separate reaction.

21. The method of claim 19 or 20, wherein the one or more pools comprise:
(a) one or more pools as defined in claim 2(I); and/or
(b) one or more pools as defined in claim 2(II); and/or
(c) one or more pools as defined in claim 17 or 18.

22. The method of any one of claims 19 to 21, wherein each of the one or more pools comprises fragments derived from a different microbial protein.

23. The method of any one of claims 19 to 22, wherein the method further comprises contacting the sample with a pool of fragments derived from a protein from the microbe and detecting in vitro the presence or absence of an immune response to the pool, wherein the fragments in the pool form a protein fragment library encompassing at least 80% of the sequence of the protein.

24. The method of any one of claims 19 to 23, wherein the method further comprises, in a separate reaction, contacting the sample with a pool of fragments derived from a protein from a microbe in the same family as the microbe from which the microbial protein is derived and detecting in vitro the presence or absence of an immune response to the pool, wherein the fragments in the pool form a protein fragment library encompassing at least 80% of the sequence of the protein.


SEQUENCE LISTING (744 sequences)

SEQ ID NO: 1 - protein, 15 aa, SARS-CoV-2
MFVFLVLLPLVSSQC

SEQ ID NO: 2 - protein, 15 aa, SARS-CoV-2
LVLLPLVSSQCVNLT

SEQ ID NO: 3 - protein, 15 aa, SARS-CoV-2
PLVSSQCVNLTTRTQ

SEQ ID NO: 4 - protein, 15 aa, SARS-CoV-2
SQCVNLTTRTQLPPA

SEQ ID NO: 5 - protein, 15 aa, SARS-CoV-2
NLTTRTQLPPAYTNS

SEQ ID NO: 6 - protein, 15 aa, SARS-CoV-2
RTQLPPAYTNSFTRG

SEQ ID NO: 7 - protein, 15 aa, SARS-CoV-2
PPAYTNSFTRGVYYP

SEQ ID NO: 8 - protein, 15 aa, SARS-CoV-2
TNSFTRGVYYPDKVF

SEQ ID NO: 9 - protein, 15 aa, SARS-CoV-2
TRGVYYPDKVFRSSV

SEQ ID NO: 10 - protein, 15 aa, SARS-CoV-2
YYPDKVFRSSVLHST

SEQ ID NO: 11 - protein, 15 aa, SARS-CoV-2
KVFRSSVLHSTQDLF

SEQ ID NO: 12 - protein, 15 aa, SARS-CoV-2
SSVLHSTQDLFLPFF

SEQ ID NO: 13 - protein, 15 aa, SARS-CoV-2
HSTQDLFLPFFSNVT

SEQ ID NO: 14 - protein, 15 aa, SARS-CoV-2
DLFLPFFSNVTWFHA

SEQ ID NO: 15 - protein, 15 aa, SARS-CoV-2
PFFSNVTWFHAIHVS

SEQ ID NO: 16 - protein, 15 aa, SARS-CoV-2
NVTWFHAIHVSGTNG

SEQ ID NO: 17 - protein, 15 aa, SARS-CoV-2
FHAIHVSGTNGTKRF

SEQ ID NO: 18 - protein, 15 aa, SARS-CoV-2
HVSGTNGTKRFDNPV

SEQ ID NO: 19 - protein, 15 aa, SARS-CoV-2
TNGTKRFDNPVLPFN

SEQ ID NO: 20 - protein, 15 aa, SARS-CoV-2
KRFDNPVLPFNDGVY

SEQ ID NO: 21 - protein, 15 aa, SARS-CoV-2
NPVLPFNDGVYFAST

SEQ ID NO: 22 - protein, 15 aa, SARS-CoV-2
PFNDGVYFASTEKSN

SEQ ID NO: 23 - protein, 15 aa, SARS-CoV-2
GVYFASTEKSNIIRG

SEQ ID NO: 24 - protein, 15 aa, SARS-CoV-2
ASTEKSNIIRGWIFG

SEQ ID NO: 25 - protein, 15 aa, SARS-CoV-2
KSNIIRGWIFGTTLD

SEQ ID NO: 26 - protein, 15 aa, SARS-CoV-2
IRGWIFGTTLDSKTQ

SEQ ID NO: 27 - protein, 15 aa, SARS-CoV-2
IFGTTLDSKTQSLLI

SEQ ID NO: 28 - protein, 15 aa, SARS-CoV-2
TLDSKTQSLLIVNNA

SEQ ID NO: 29 - protein, 15 aa, SARS-CoV-2
KTQSLLIVNNATNVV

SEQ ID NO: 30 - protein, 15 aa, SARS-CoV-2
LLIVNNATNVVIKVC

SEQ ID NO: 31 - protein, 15 aa, SARS-CoV-2
NNATNVVIKVCEFQF

SEQ ID NO: 32 - protein, 15 aa, SARS-CoV-2
NVVIKVCEFQFCNDP

SEQ ID NO: 33 - protein, 15 aa, SARS-CoV-2
KVCEFQFCNDPFLGV

SEQ ID NO: 34 - protein, 15 aa, SARS-CoV-2
FQFCNDPFLGVYYHK

SEQ ID NO: 35 - protein, 15 aa, SARS-CoV-2
NDPFLGVYYHKNNKS

SEQ ID NO: 36 - protein, 15 aa, SARS-CoV-2
LGVYYHKNNKSWMES

SEQ ID NO: 37 - protein, 15 aa, SARS-CoV-2
YHKNNKSWMESEFRV

SEQ ID NO: 38 - protein, 15 aa, SARS-CoV-2
NKSWMESEFRVYSSA

SEQ ID NO: 39 - protein, 15 aa, SARS-CoV-2
MESEFRVYSSANNCT

SEQ ID NO: 40 - protein, 15 aa, SARS-CoV-2
FRVYSSANNCTFEYV

SEQ ID NO: 41 - protein, 15 aa, SARS-CoV-2
SSANNCTFEYVSQPF

SEQ ID NO: 42 - protein, 15 aa, SARS-CoV-2
NCTFEYVSQPFLMDL

SEQ ID NO: 43 - protein, 15 aa, SARS-CoV-2
EYVSQPFLMDLEGKQ

SEQ ID NO: 44 - protein, 15 aa, SARS-CoV-2
QPFLMDLEGKQGNFK

SEQ ID NO: 45 - protein, 15 aa, SARS-CoV-2
MDLEGKQGNFKNLRE

SEQ ID NO: 46 - protein, 15 aa, SARS-CoV-2
GKQGNFKNLREFVFK

SEQ ID NO: 47 - protein, 15 aa, SARS-CoV-2
NFKNLREFVFKNIDG

SEQ ID NO: 48 - protein, 15 aa, SARS-CoV-2
LREFVFKNIDGYFKI

SEQ ID NO: 49 - protein, 15 aa, SARS-CoV-2
VFKNIDGYFKIYSKH

SEQ ID NO: 50 - protein, 15 aa, SARS-CoV-2
IDGYFKIYSKHTPIN

SEQ ID NO: 51 - protein, 15 aa, SARS-CoV-2
FKIYSKHTPINLVRD

SEQ ID NO: 52 - protein, 15 aa, SARS-CoV-2
SKHTPINLVRDLPQG

SEQ ID NO: 53 - protein, 15 aa, SARS-CoV-2
PINLVRDLPQGFSAL

SEQ ID NO: 54 - protein, 15 aa, SARS-CoV-2
VRDLPQGFSALEPLV

SEQ ID NO: 55 - protein, 15 aa, SARS-CoV-2
PQGFSALEPLVDLPI

SEQ ID NO: 56 - protein, 15 aa, SARS-CoV-2
SALEPLVDLPIGINI

SEQ ID NO: 57 - protein, 15 aa, SARS-CoV-2
PLVDLPIGINITRFQ

SEQ ID NO: 58 - protein, 15 aa, SARS-CoV-2
LPIGINITRFQTLLA

SEQ ID NO: 59 - protein, 15 aa, SARS-CoV-2
INITRFQTLLALHRS

SEQ ID NO: 60 - protein, 15 aa, SARS-CoV-2
RFQTLLALHRSYLTP

SEQ ID NO: 61 - protein, 15 aa, SARS-CoV-2
LLALHRSYLTPGDSS

SEQ ID NO: 62 - protein, 15 aa, SARS-CoV-2
HRSYLTPGDSSSGWT

SEQ ID NO: 63 - protein, 15 aa, SARS-CoV-2
LTPGDSSSGWTAGAA

SEQ ID NO: 64 - protein, 15 aa, SARS-CoV-2
DSSSGWTAGAAAYYV

SEQ ID NO: 65 - protein, 15 aa, SARS-CoV-2
GWTAGAAAYYVGYLQ

SEQ ID NO: 66 - protein, 15 aa, SARS-CoV-2
GAAAYYVGYLQPRTF

SEQ ID NO: 67 - protein, 15 aa, SARS-CoV-2
YYVGYLQPRTFLLKY

SEQ ID NO: 68 - protein, 15 aa, SARS-CoV-2
YLQPRTFLLKYNENG

SEQ ID NO: 69 - protein, 15 aa, SARS-CoV-2
RTFLLKYNENGTITD

SEQ ID NO: 70 - protein, 15 aa, SARS-CoV-2
LKYNENGTITDAVDC

SEQ ID NO: 71 - protein, 15 aa, SARS-CoV-2
ENGTITDAVDCALDP

SEQ ID NO: 72 - protein, 15 aa, SARS-CoV-2
ITDAVDCALDPLSET

SEQ ID NO: 73 - protein, 15 aa, SARS-CoV-2
VDCALDPLSETKCTL

SEQ ID NO: 74 - protein, 15 aa, SARS-CoV-2
LDPLSETKCTLKSFT

SEQ ID NO: 75 - protein, 15 aa, SARS-CoV-2
SETKCTLKSFTVEKG

SEQ ID NO: 76 - protein, 15 aa, SARS-CoV-2
CTLKSFTVEKGIYQT

SEQ ID NO: 77 - protein, 15 aa, SARS-CoV-2
SFTVEKGIYQTSNFR

SEQ ID NO: 78 - protein, 15 aa, SARS-CoV-2
EKGIYQTSNFRVQPT

SEQ ID NO: 79 - protein, 15 aa, SARS-CoV-2
YQTSNFRVQPTESIV

SEQ ID NO: 80 - protein, 15 aa, SARS-CoV-2
NFRVQPTESIVRFPN

SEQ ID NO: 81 - protein, 15 aa, SARS-CoV-2
QPTESIVRFPNITNL

SEQ ID NO: 82 - protein, 15 aa, SARS-CoV-2
SIVRFPNITNLCPFG

SEQ ID NO: 83 - protein, 15 aa, SARS-CoV-2
FPNITNLCPFGEVFN

SEQ ID NO: 84 - protein, 15 aa, SARS-CoV-2
TNLCPFGEVFNATRF

SEQ ID NO: 85 - protein, 15 aa, SARS-CoV-2
PFGEVFNATRFASVY

SEQ ID NO: 86 - protein, 15 aa, SARS-CoV-2
VFNATRFASVYAWNR

SEQ ID NO: 87 - protein, 15 aa, SARS-CoV-2
TRFASVYAWNRKRIS

SEQ ID NO: 88 - protein, 15 aa, SARS-CoV-2
SVYAWNRKRISNCVA

SEQ ID NO: 89 - protein, 15 aa, SARS-CoV-2
WNRKRISNCVADYSV

SEQ ID NO: 90 - protein, 15 aa, SARS-CoV-2
RISNCVADYSVLYNS

SEQ ID NO: 91 - protein, 15 aa, SARS-CoV-2
CVADYSVLYNSASFS

SEQ ID NO: 92 - protein, 15 aa, SARS-CoV-2
YSVLYNSASFSTFKC

SEQ ID NO: 93 - protein, 15 aa, SARS-CoV-2
YNSASFSTFKCYGVS

SEQ ID NO: 94 - protein, 15 aa, SARS-CoV-2
SFSTFKCYGVSPTKL

SEQ ID NO: 95 - protein, 15 aa, SARS-CoV-2
FKCYGVSPTKLNDLC

SEQ ID NO: 96 - protein, 15 aa, SARS-CoV-2
GVSPTKLNDLCFTNV

SEQ ID NO: 97 - protein, 15 aa, SARS-CoV-2
TKLNDLCFTNVYADS

SEQ ID NO: 98 - protein, 15 aa, SARS-CoV-2
DLCFTNVYADSFVIR

SEQ ID NO: 99 - protein, 15 aa, SARS-CoV-2
TNVYADSFVIRGDEV

SEQ ID NO: 100 - protein, 15 aa, SARS-CoV-2
ADSFVIRGDEVRQIA

SEQ ID NO: 101 - protein, 15 aa, SARS-CoV-2
VIRGDEVRQIAPGQT

SEQ ID NO: 102 - protein, 15 aa, SARS-CoV-2
DEVRQIAPGQTGKIA

SEQ ID NO: 103 - protein, 15 aa, SARS-CoV-2
QIAPGQTGKIADYNY

SEQ ID NO: 104 - protein, 15 aa, SARS-CoV-2
GQTGKIADYNYKLPD

SEQ ID NO: 105 - protein, 15 aa, SARS-CoV-2
KIADYNYKLPDDFTG

SEQ ID NO: 106 - protein, 15 aa, SARS-CoV-2
YNYKLPDDFTGCVIA

SEQ ID NO: 107 - protein, 15 aa, SARS-CoV-2
LPDDFTGCVIAWNSN

SEQ ID NO: 108 - protein, 15 aa, SARS-CoV-2
FTGCVIAWNSNNLDS

SEQ ID NO: 109 - protein, 15 aa, SARS-CoV-2
VIAWNSNNLDSKVGG

SEQ ID NO: 110 - protein, 15 aa, SARS-CoV-2
NSNNLDSKVGGNYNY

SEQ ID NO: 111 - protein, 15 aa, SARS-CoV-2
LDSKVGGNYNYLYRL

SEQ ID NO: 112 - protein, 15 aa, SARS-CoV-2
VGGNYNYLYRLFRKS

SEQ ID NO: 113 - protein, 15 aa, SARS-CoV-2
YNYLYRLFRKSNLKP

SEQ ID NO: 114 - protein, 15 aa, SARS-CoV-2
YRLFRKSNLKPFERD

SEQ ID NO: 115 - protein, 15 aa, SARS-CoV-2
RKSNLKPFERDISTE

SEQ ID NO: 116 - protein, 15 aa, SARS-CoV-2
LKPFERDISTEIYQA

SEQ ID NO: 117 - protein, 15 aa, SARS-CoV-2
ERDISTEIYQAGSTP

SEQ ID NO: 118 - protein, 15 aa, SARS-CoV-2
STEIYQAGSTPCNGV

SEQ ID NO: 119 - protein, 15 aa, SARS-CoV-2
YQAGSTPCNGVEGFN

SEQ ID NO: 120 - protein, 15 aa, SARS-CoV-2
STPCNGVEGFNCYFP

SEQ ID NO: 121 - protein, 15 aa, SARS-CoV-2
NGVEGFNCYFPLQSY

SEQ ID NO: 122 - protein, 15 aa, SARS-CoV-2
GFNCYFPLQSYGFQP

SEQ ID NO: 123 - protein, 15 aa, SARS-CoV-2
YFPLQSYGFQPTNGV

SEQ ID NO: 124 - protein, 15 aa, SARS-CoV-2
QSYGFQPTNGVGYQP

SEQ ID NO: 125 - protein, 15 aa, SARS-CoV-2
FQPTNGVGYQPYRVV

SEQ ID NO: 126 - protein, 15 aa, SARS-CoV-2
NGVGYQPYRVVVLSF

SEQ ID NO: 127 - protein, 15 aa, SARS-CoV-2
YQPYRVVVLSFELLH

SEQ ID NO: 128 - protein, 15 aa, SARS-CoV-2
RVVVLSFELLHAPAT

SEQ ID NO: 129 - protein, 15 aa, SARS-CoV-2
LSFELLHAPATVCGP

SEQ ID NO: 130 - protein, 15 aa, SARS-CoV-2
LLHAPATVCGPKKST

SEQ ID NO: 131 - protein, 15 aa, SARS-CoV-2
PATVCGPKKSTNLVK

SEQ ID NO: 132 - protein, 15 aa, SARS-CoV-2
CGPKKSTNLVKNKCV

SEQ ID NO: 133 - protein, 15 aa, SARS-CoV-2
KSTNLVKNKCVNFNF

SEQ ID NO: 134 - protein, 15 aa, SARS-CoV-2
LVKNKCVNFNFNGLT

SEQ ID NO: 135 - protein, 15 aa, SARS-CoV-2
KCVNFNFNGLTGTGV

SEQ ID NO: 136 - protein, 15 aa, SARS-CoV-2
FNFNGLTGTGVLTES

SEQ ID NO: 137 - protein, 15 aa, SARS-CoV-2
GLTGTGVLTESNKKF

SEQ ID NO: 138 - protein, 15 aa, SARS-CoV-2
TGVLTESNKKFLPFQ

SEQ ID NO: 139 - protein, 15 aa, SARS-CoV-2
TESNKKFLPFQQFGR

SEQ ID NO: 140 - protein, 15 aa, SARS-CoV-2
KKFLPFQQFGRDIAD

SEQ ID NO: 141 - protein, 15 aa, SARS-CoV-2
PFQQFGRDIADTTDA

SEQ ID NO: 142 - protein, 15 aa, SARS-CoV-2
FGRDIADTTDAVRDP

SEQ ID NO: 143 - protein, 15 aa, SARS-CoV-2
IADTTDAVRDPQTLE

SEQ ID NO: 144 - protein, 15 aa, SARS-CoV-2
TDAVRDPQTLEILDI

SEQ ID NO: 145 - protein, 15 aa, SARS-CoV-2
RDPQTLEILDITPCS

SEQ ID NO: 146 - protein, 15 aa, SARS-CoV-2
TLEILDITPCSFGGV

SEQ ID NO: 147 - protein, 15 aa, SARS-CoV-2
LDITPCSFGGVSVIT

SEQ ID NO: 148 - protein, 15 aa, SARS-CoV-2
PCSFGGVSVITPGTN

SEQ ID NO: 149 - protein, 15 aa, SARS-CoV-2
GGVSVITPGTNTSNQ

SEQ ID NO: 150 - protein, 15 aa, SARS-CoV-2
VITPGTNTSNQVAVL

SEQ ID NO: 151 - protein, 15 aa, SARS-CoV-2
GTNTSNQVAVLYQDV

SEQ ID NO: 152 - protein, 15 aa, SARS-CoV-2
SNQVAVLYQDVNCTE

SEQ ID NO: 153 - protein, 15 aa, SARS-CoV-2
AVLYQDVNCTEVPVA

SEQ ID NO: 154 - protein, 15 aa, SARS-CoV-2
QDVNCTEVPVAIHAD

SEQ ID NO: 155 - protein, 15 aa, SARS-CoV-2
CTEVPVAIHADQLTP

SEQ ID NO: 156 - protein, 15 aa, SARS-CoV-2
PVAIHADQLTPTWRV

SEQ ID NO: 157 - protein, 15 aa, SARS-CoV-2
HADQLTPTWRVYSTG

SEQ ID NO: 158 - protein, 15 aa, SARS-CoV-2
LTPTWRVYSTGSNVF

SEQ ID NO: 159 - protein, 15 aa, SARS-CoV-2
WRVYSTGSNVFQTRA

SEQ ID NO: 160 - protein, 15 aa, SARS-CoV-2
STGSNVFQTRAGCLI

SEQ ID NO: 161 - protein, 15 aa, SARS-CoV-2
NVFQTRAGCLIGAEH

SEQ ID NO: 162 - protein, 15 aa, SARS-CoV-2
TRAGCLIGAEHVNNS

SEQ ID NO: 163 - protein, 15 aa, SARS-CoV-2
CLIGAEHVNNSYECD

SEQ ID NO: 164 - protein, 15 aa, SARS-CoV-2
AEHVNNSYECDIPIG

SEQ ID NO: 165 - protein, 15 aa, SARS-CoV-2
NNSYECDIPIGAGIC

SEQ ID NO: 166 - protein, 15 aa, SARS-CoV-2
ECDIPIGAGICASYQ

SEQ ID NO: 167 - protein, 15 aa, SARS-CoV-2
PIGAGICASYQTQTN

SEQ ID NO: 168 - protein, 15 aa, SARS-CoV-2
GICASYQTQTNSPRR

SEQ ID NO: 169 - protein, 15 aa, SARS-CoV-2
SYQTQTNSPRRARSV

SEQ ID NO: 170 - protein, 15 aa, SARS-CoV-2
QTNSPRRARSVASQS

SEQ ID NO: 171 - protein, 15 aa, SARS-CoV-2
PRRARSVASQSIIAY

SEQ ID NO: 172 - protein, 15 aa, SARS-CoV-2
RSVASQSIIAYTMSL

SEQ ID NO: 173 - protein, 15 aa, SARS-CoV-2
SQSIIAYTMSLGAEN

SEQ ID NO: 174 - protein, 15 aa, SARS-CoV-2
IAYTMSLGAENSVAY

SEQ ID NO: 175 - protein, 15 aa, SARS-CoV-2
MSLGAENSVAYSNNS

SEQ ID NO: 176 - protein, 15 aa, SARS-CoV-2
AENSVAYSNNSIAIP

SEQ ID NO: 177 - protein, 15 aa, SARS-CoV-2
VAYSNNSIAIPTNFT

SEQ ID NO: 178 - protein, 15 aa, SARS-CoV-2
NNSIAIPTNFTISVT

SEQ ID NO: 179 - protein, 15 aa, SARS-CoV-2
AIPTNFTISVTTEIL

SEQ ID NO: 180 - protein, 15 aa, SARS-CoV-2
NFTISVTTEILPVSM

SEQ ID NO: 181 - protein, 15 aa, SARS-CoV-2
SVTTEILPVSMTKTS

SEQ ID NO: 182 - protein, 15 aa, SARS-CoV-2
EILPVSMTKTSVDCT

SEQ ID NO: 183 - protein, 15 aa, SARS-CoV-2
VSMTKTSVDCTMYIC

SEQ ID NO: 184 - protein, 15 aa, SARS-CoV-2
KTSVDCTMYICGDST

SEQ ID NO: 185 - protein, 15 aa, SARS-CoV-2
DCTMYICGDSTECSN

SEQ ID NO: 186 - protein, 15 aa, SARS-CoV-2
YICGDSTECSNLLLQ

SEQ ID NO: 187 - protein, 15 aa, SARS-CoV-2
DSTECSNLLLQYGSF

SEQ ID NO: 188 - protein, 15 aa, SARS-CoV-2
CSNLLLQYGSFCTQL

SEQ ID NO: 189 - protein, 15 aa, SARS-CoV-2
LLQYGSFCTQLNRAL

SEQ ID NO: 190 - protein, 15 aa, SARS-CoV-2
GSFCTQLNRALTGIA

SEQ ID NO: 191 - protein, 15 aa, SARS-CoV-2
TQLNRALTGIAVEQD

SEQ ID NO: 192 - protein, 15 aa, SARS-CoV-2
RALTGIAVEQDKNTQ

SEQ ID NO: 193 - protein, 15 aa, SARS-CoV-2
GIAVEQDKNTQEVFA

SEQ ID NO: 194 - protein, 15 aa, SARS-CoV-2
EQDKNTQEVFAQVKQ

SEQ ID NO: 195 - protein, 15 aa, SARS-CoV-2
NTQEVFAQVKQIYKT

SEQ ID NO: 196 - protein, 15 aa, SARS-CoV-2
VFAQVKQIYKTPPIK

SEQ ID NO: 197 - protein, 15 aa, SARS-CoV-2
VKQIYKTPPIKDFGG

SEQ ID NO: 198 - protein, 15 aa, SARS-CoV-2
YKTPPIKDFGGFNFS

SEQ ID NO: 199 - protein, 15 aa, SARS-CoV-2
PIKDFGGFNFSQILP

SEQ ID NO: 200 - protein, 15 aa, SARS-CoV-2
FGGFNFSQILPDPSK

SEQ ID NO: 201 - protein, 15 aa, SARS-CoV-2
NFSQILPDPSKPSKR

SEQ ID NO: 202 - protein, 15 aa, SARS-CoV-2
ILPDPSKPSKRSFIE

SEQ ID NO: 203 - protein, 15 aa, SARS-CoV-2
PSKPSKRSFIEDLLF

SEQ ID NO: 204 - protein, 15 aa, SARS-CoV-2
SKRSFIEDLLFNKVT

SEQ ID NO: 205 - protein, 15 aa, SARS-CoV-2
FIEDLLFNKVTLADA

SEQ ID NO: 206 - protein, 15 aa, SARS-CoV-2
LLFNKVTLADAGFIK

SEQ ID NO: 207 - protein, 15 aa, SARS-CoV-2
KVTLADAGFIKQYGD

SEQ ID NO: 208 - protein, 15 aa, SARS-CoV-2
ADAGFIKQYGDCLGD

SEQ ID NO: 209 - protein, 15 aa, SARS-CoV-2
FIKQYGDCLGDIAAR

SEQ ID NO: 210 - protein, 15 aa, SARS-CoV-2
YGDCLGDIAARDLIC

SEQ ID NO: 211 - protein, 15 aa, SARS-CoV-2
LGDIAARDLICAQKF

SEQ ID NO: 212 - protein, 15 aa, SARS-CoV-2
AARDLICAQKFNGLT

SEQ ID NO: 213 - protein, 15 aa, SARS-CoV-2
LICAQKFNGLTVLPP

SEQ ID NO: 214 - protein, 15 aa, SARS-CoV-2
QKFNGLTVLPPLLTD

SEQ ID NO: 215 - protein, 15 aa, SARS-CoV-2
GLTVLPPLLTDEMIA

SEQ ID NO: 216 - protein, 15 aa, SARS-CoV-2
LPPLLTDEMIAQYTS

SEQ ID NO: 217 - protein, 15 aa, SARS-CoV-2
LTDEMIAQYTSALLA

SEQ ID NO: 218 - protein, 15 aa, SARS-CoV-2
MIAQYTSALLAGTIT

SEQ ID NO: 219 - protein, 15 aa, SARS-CoV-2
YTSALLAGTITSGWT

SEQ ID NO: 220 - protein, 15 aa, SARS-CoV-2
LLAGTITSGWTFGAG

SEQ ID NO: 221 - protein, 15 aa, SARS-CoV-2
TITSGWTFGAGAALQ

SEQ ID NO: 222 - protein, 15 aa, SARS-CoV-2
GWTFGAGAALQIPFA

SEQ ID NO: 223 - protein, 15 aa, SARS-CoV-2
GAGAALQIPFAMQMA

SEQ ID NO: 224 - protein, 15 aa, SARS-CoV-2
ALQIPFAMQMAYRFN

SEQ ID NO: 225 - protein, 15 aa, SARS-CoV-2
PFAMQMAYRFNGIGV

SEQ ID NO: 226 - protein, 15 aa, SARS-CoV-2
QMAYRFNGIGVTQNV

SEQ ID NO: 227 - protein, 15 aa, SARS-CoV-2
RFNGIGVTQNVLYEN

SEQ ID NO: 228 - protein, 15 aa, SARS-CoV-2
IGVTQNVLYENQKLI

SEQ ID NO: 229 - protein, 15 aa, SARS-CoV-2
QNVLYENQKLIANQF

SEQ ID NO: 230 - protein, 15 aa, SARS-CoV-2
YENQKLIANQFNSAI

SEQ ID NO: 231 - protein, 15 aa, SARS-CoV-2
KLIANQFNSAIGKIQ

SEQ ID NO: 232 - protein, 15 aa, SARS-CoV-2
NQFNSAIGKIQDSLS

SEQ ID NO: 233 - protein, 15 aa, SARS-CoV-2
SAIGKIQDSLSSTAS

SEQ ID NO: 234 - protein, 15 aa, SARS-CoV-2
KIQDSLSSTASALGK

SEQ ID NO: 235 - protein, 15 aa, SARS-CoV-2
SLSSTASALGKLQDV

SEQ ID NO: 236 - protein, 15 aa, SARS-CoV-2
TASALGKLQDVVNQN

SEQ ID NO: 237 - protein, 15 aa, SARS-CoV-2
LGKLQDVVNQNAQAL

SEQ ID NO: 238 - protein, 15 aa, SARS-CoV-2
QDVVNQNAQALNTLV

SEQ ID NO: 239 - protein, 15 aa, SARS-CoV-2
NQNAQALNTLVKQLS

SEQ ID NO: 240 - protein, 15 aa, SARS-CoV-2
QALNTLVKQLSSNFG

SEQ ID NO: 241 - protein, 15 aa, SARS-CoV-2
TLVKQLSSNFGAISS

SEQ ID NO: 242 - protein, 15 aa, SARS-CoV-2
QLSSNFGAISSVLND

SEQ ID NO: 243 - protein, 15 aa, SARS-CoV-2
NFGAISSVLNDILSR

SEQ ID NO: 244 - protein, 15 aa, SARS-CoV-2
ISSVLNDILSRLDKV

SEQ ID NO: 245 - protein, 15 aa, SARS-CoV-2
LNDILSRLDKVEAEV

SEQ ID NO: 246 - protein, 15 aa, SARS-CoV-2
LSRLDKVEAEVQIDR

SEQ ID NO: 247 - protein, 15 aa, SARS-CoV-2
DKVEAEVQIDRLITG

SEQ ID NO: 248 - protein, 15 aa, SARS-CoV-2
AEVQIDRLITGRLQS

SEQ ID NO: 249 - protein, 15 aa, SARS-CoV-2
IDRLITGRLQSLQTY

SEQ ID NO: 250 - protein, 15 aa, SARS-CoV-2
ITGRLQSLQTYVTQQ

SEQ ID NO: 251 - protein, 15 aa, SARS-CoV-2
LQSLQTYVTQQLIRA

SEQ ID NO: 252 - protein, 15 aa, SARS-CoV-2
QTYVTQQLIRAAEIR

SEQ ID NO: 253 - protein, 15 aa, SARS-CoV-2
TQQLIRAAEIRASAN

SEQ ID NO: 254 - protein, 15 aa, SARS-CoV-2
IRAAEIRASANLAAT

SEQ ID NO: 255 - protein, 15 aa, SARS-CoV-2
EIRASANLAATKMSE

SEQ ID NO: 256 - protein, 15 aa, SARS-CoV-2
SANLAATKMSECVLG

SEQ ID NO: 257 - protein, 15 aa, SARS-CoV-2
AATKMSECVLGQSKR

SEQ ID NO: 258 - protein, 15 aa, SARS-CoV-2
MSECVLGQSKRVDFC

SEQ ID NO: 259 - protein, 15 aa, SARS-CoV-2
VLGQSKRVDFCGKGY

SEQ ID NO: 260 - protein, 15 aa, SARS-CoV-2
SKRVDFCGKGYHLMS

SEQ ID NO: 261 - protein, 15 aa, SARS-CoV-2
DFCGKGYHLMSFPQS

SEQ ID NO: 262 - protein, 15 aa, SARS-CoV-2
KGYHLMSFPQSAPHG

SEQ ID NO: 263 - protein, 15 aa, SARS-CoV-2
LMSFPQSAPHGVVFL

SEQ ID NO: 264 - protein, 15 aa, SARS-CoV-2
PQSAPHGVVFLHVTY

SEQ ID NO: 265 - protein, 15 aa, SARS-CoV-2
PHGVVFLHVTYVPAQ

SEQ ID NO: 266 - protein, 15 aa, SARS-CoV-2
VFLHVTYVPAQEKNF

SEQ ID NO: 267 - protein, 15 aa, SARS-CoV-2
VTYVPAQEKNFTTAP

SEQ ID NO: 268 - protein, 15 aa, SARS-CoV-2
PAQEKNFTTAPAICH

SEQ ID NO: 269 - protein, 15 aa, SARS-CoV-2
KNFTTAPAICHDGKA

SEQ ID NO: 270 - protein, 15 aa, SARS-CoV-2
TAPAICHDGKAHFPR

SEQ ID NO: 271 - protein, 15 aa, SARS-CoV-2
ICHDGKAHFPREGVF

SEQ ID NO: 272 - protein, 15 aa, SARS-CoV-2
GKAHFPREGVFVSNG

SEQ ID NO: 273 - protein, 15 aa, SARS-CoV-2
FPREGVFVSNGTHWF

SEQ ID NO: 274 - protein, 15 aa, SARS-CoV-2
GVFVSNGTHWFVTQR

SEQ ID NO: 275 - protein, 15 aa, SARS-CoV-2
SNGTHWFVTQRNFYE

SEQ ID NO: 276 - protein, 15 aa, SARS-CoV-2
HWFVTQRNFYEPQII

SEQ ID NO: 277 - protein, 15 aa, SARS-CoV-2
TQRNFYEPQIITTDN

SEQ ID NO: 278 - protein, 15 aa, SARS-CoV-2
FYEPQIITTDNTFVS

SEQ ID NO: 279 - protein, 15 aa, SARS-CoV-2
QIITTDNTFVSGNCD

SEQ ID NO: 280 - protein, 15 aa, SARS-CoV-2
TDNTFVSGNCDVVIG

SEQ ID NO: 281 - protein, 15 aa, SARS-CoV-2
FVSGNCDVVIGIVNN

SEQ ID NO: 282 - protein, 15 aa, SARS-CoV-2
NCDVVIGIVNNTVYD

SEQ ID NO: 283 - protein, 15 aa, SARS-CoV-2
VIGIVNNTVYDPLQP

SEQ ID NO: 284 - protein, 15 aa, SARS-CoV-2
VNNTVYDPLQPELDS

SEQ ID NO: 285 - protein, 15 aa, SARS-CoV-2
VYDPLQPELDSFKEE

SEQ ID NO: 286 - protein, 15 aa, SARS-CoV-2
LQPELDSFKEELDKY

SEQ ID NO: 287 - protein, 15 aa, SARS-CoV-2
LDSFKEELDKYFKNH

SEQ ID NO: 288 - protein, 15 aa, SARS-CoV-2
KEELDKYFKNHTSPD

SEQ ID NO: 289 - protein, 15 aa, SARS-CoV-2
DKYFKNHTSPDVDLG

SEQ ID NO: 290 - protein, 15 aa, SARS-CoV-2
KNHTSPDVDLGDISG

SEQ ID NO: 291 - protein, 15 aa, SARS-CoV-2
SPDVDLGDISGINAS

SEQ ID NO: 292 - protein, 15 aa, SARS-CoV-2
DLGDISGINASVVNI

SEQ ID NO: 293 - protein, 15 aa, SARS-CoV-2
ISGINASVVNIQKEI

SEQ ID NO: 294 - protein, 15 aa, SARS-CoV-2
NASVVNIQKEIDRLN

SEQ ID NO: 295 - protein, 15 aa, SARS-CoV-2
VNIQKEIDRLNEVAK

SEQ ID NO: 296 - protein, 15 aa, SARS-CoV-2
KEIDRLNEVAKNLNE

SEQ ID NO: 297 - protein, 15 aa, SARS-CoV-2
RLNEVAKNLNESLID

SEQ ID NO: 298 - protein, 15 aa, SARS-CoV-2
VAKNLNESLIDLQEL

SEQ ID NO: 299 - protein, 15 aa, SARS-CoV-2
LNESLIDLQELGKYE

SEQ ID NO: 300 - protein, 15 aa, SARS-CoV-2
LIDLQELGKYEQYIK

SEQ ID NO: 301 - protein, 15 aa, SARS-CoV-2
QELGKYEQYIKWPWY

SEQ ID NO: 302 - protein, 15 aa, SARS-CoV-2
KYEQYIKWPWYIWLG

SEQ ID NO: 303 - protein, 15 aa, SARS-CoV-2
YIKWPWYIWLGFIAG

SEQ ID NO: 304 - protein, 15 aa, SARS-CoV-2
PWYIWLGFIAGLIAI

SEQ ID NO: 305 - protein, 15 aa, SARS-CoV-2
WLGFIAGLIAIVMVT

SEQ ID NO: 306 - protein, 15 aa, SARS-CoV-2
IAGLIAIVMVTIMLC

SEQ ID NO: 307 - protein, 15 aa, SARS-CoV-2
IAIVMVTIMLCCMTS

SEQ ID NO: 308 - protein, 15 aa, SARS-CoV-2
MVTIMLCCMTSCCSC

SEQ ID NO: 309 - protein, 15 aa, SARS-CoV-2
MLCCMTSCCSCLKGC

SEQ ID NO: 310 - protein, 15 aa, SARS-CoV-2
MTSCCSCLKGCCSCG

SEQ ID NO: 311 - protein, 15 aa, SARS-CoV-2
CSCLKGCCSCGSCCK

SEQ ID NO: 312 - protein, 15 aa, SARS-CoV-2
KGCCSCGSCCKFDED

SEQ ID NO: 313 - protein, 15 aa, SARS-CoV-2
SCGSCCKFDEDDSEP

SEQ ID NO: 314 - protein, 15 aa, SARS-CoV-2
CCKFDEDDSEPVLKG

SEQ ID NO: 315 - protein, 15 aa, SARS-CoV-2
DEDDSEPVLKGVKLH

SEQ ID NO: 316 - protein, 15 aa, SARS-CoV-2
MADSNGTITVEELKK

SEQ ID NO: 317 - protein, 15 aa, SARS-CoV-2
NGTITVEELKKLLEQ

SEQ ID NO: 318 - protein, 15 aa, SARS-CoV-2
TVEELKKLLEQWNLV

SEQ ID NO: 319 - protein, 15 aa, SARS-CoV-2
LKKLLEQWNLVIGFL

SEQ ID NO: 320 - protein, 15 aa, SARS-CoV-2
LEQWNLVIGFLFLTW

SEQ ID NO: 321 - protein, 15 aa, SARS-CoV-2
NLVIGFLFLTWICLL

SEQ ID NO: 322 - protein, 15 aa, SARS-CoV-2
GFLFLTWICLLQFAY

SEQ ID NO: 323 - protein, 15 aa, SARS-CoV-2
LTWICLLQFAYANRN

SEQ ID NO: 324 - protein, 15 aa, SARS-CoV-2
CLLQFAYANRNRFLY

SEQ ID NO: 325 - protein, 15 aa, SARS-CoV-2
FAYANRNRFLYIIKL

SEQ ID NO: 326 - protein, 15 aa, SARS-CoV-2
NRNRFLYIIKLIFLW

SEQ ID NO: 327 - protein, 15 aa, SARS-CoV-2
FLYIIKLIFLWLLWP

SEQ ID NO: 328 - protein, 15 aa, SARS-CoV-2
IKLIFLWLLWPVTLA

SEQ ID NO: 329 - protein, 15 aa, SARS-CoV-2
FLWLLWPVTLACFVL

SEQ ID NO: 330 - protein, 15 aa, SARS-CoV-2
LWPVTLACFVLAAVY

SEQ ID NO: 331 - protein, 15 aa, SARS-CoV-2
TLACFVLAAVYRINW

SEQ ID NO: 332 - protein, 15 aa, SARS-CoV-2
FVLAAVYRINWITGG

SEQ ID NO: 333 - protein, 15 aa, SARS-CoV-2
AVYRINWITGGIAIA

SEQ ID NO: 334 - protein, 15 aa, SARS-CoV-2
INWITGGIAIAMACL

SEQ ID NO: 335 - protein, 15 aa, SARS-CoV-2
TGGIAIAMACLVGLM

SEQ ID NO: 336 - protein, 15 aa, SARS-CoV-2
AIAMACLVGLMWLSY

SEQ ID NO: 337 - protein, 15 aa, SARS-CoV-2
ACLVGLMWLSYFIAS

SEQ ID NO: 338 - protein, 15 aa, SARS-CoV-2
GLMWLSYFIASFRLF

SEQ ID NO: 339 - protein, 15 aa, SARS-CoV-2
LSYFIASFRLFARTR

SEQ ID NO: 340 - protein, 15 aa, SARS-CoV-2
IASFRLFARTRSMWS

SEQ ID NO: 341 - protein, 15 aa, SARS-CoV-2
RLFARTRSMWSFNPE

SEQ ID NO: 342 - protein, 15 aa, SARS-CoV-2
RTRSMWSFNPETNIL

SEQ ID NO: 343 - protein, 15 aa, SARS-CoV-2
MWSFNPETNILLNVP

SEQ ID NO: 344 - protein, 15 aa, SARS-CoV-2
NPETNILLNVPLHGT

SEQ ID NO: 345 - protein, 15 aa, SARS-CoV-2
NILLNVPLHGTILTR

SEQ ID NO: 346 - protein, 15 aa, SARS-CoV-2
NVPLHGTILTRPLLE

SEQ ID NO: 347 - protein, 15 aa, SARS-CoV-2
HGTILTRPLLESELV

SEQ ID NO: 348 - protein, 15 aa, SARS-CoV-2
LTRPLLESELVIGAV

SEQ ID NO: 349 - protein, 15 aa, SARS-CoV-2
LLESELVIGAVILRG

SEQ ID NO: 350 - protein, 15 aa, SARS-CoV-2
ELVIGAVILRGHLRI

SEQ ID NO: 351 - protein, 15 aa, SARS-CoV-2
GAVILRGHLRIAGHH

SEQ ID NO: 352 - protein, 15 aa, SARS-CoV-2
LRGHLRIAGHHLGRC

SEQ ID NO: 353 - protein, 15 aa, SARS-CoV-2
LRIAGHHLGRCDIKD

SEQ ID NO: 354 - protein, 15 aa, SARS-CoV-2
GHHLGRCDIKDLPKE

SEQ ID NO: 355 - protein, 15 aa, SARS-CoV-2
GRCDIKDLPKEITVA

SEQ ID NO: 356 - protein, 15 aa, SARS-CoV-2
IKDLPKEITVATSRT

SEQ ID NO: 357 - protein, 15 aa, SARS-CoV-2
PKEITVATSRTLSYY

SEQ ID NO: 358 - protein, 15 aa, SARS-CoV-2
TVATSRTLSYYKLGA

SEQ ID NO: 359 - protein, 15 aa, SARS-CoV-2
SRTLSYYKLGASQRV

SEQ ID NO: 360 - protein, 15 aa, SARS-CoV-2
SYYKLGASQRVAGDS

SEQ ID NO: 361 - protein, 15 aa, SARS-CoV-2
LGASQRVAGDSGFAA

SEQ ID NO: 362 - protein, 15 aa, SARS-CoV-2
QRVAGDSGFAAYSRY

SEQ ID NO: 363 - protein, 15 aa, SARS-CoV-2
GDSGFAAYSRYRIGN

SEQ ID NO: 364 - protein, 15 aa, SARS-CoV-2
FAAYSRYRIGNYKLN

SEQ ID NO: 365 - protein, 15 aa, SARS-CoV-2
SRYRIGNYKLNTDHS

SEQ ID NO: 366 - protein, 15 aa, SARS-CoV-2
IGNYKLNTDHSSSSD

SEQ ID NO: 367 - protein, 15 aa, SARS-CoV-2
KLNTDHSSSSDNIAL

SEQ ID NO: 368 - protein, 15 aa, SARS-CoV-2
TDHSSSSDNIALLVQ

SEQ ID NO: 369 - protein, 15 aa, SARS-CoV-2
MSDNGPQNQRNAPRI

SEQ ID NO: 370 - protein, 15 aa, SARS-CoV-2
GPQNQRNAPRITFGG

SEQ ID NO: 371 - protein, 15 aa, SARS-CoV-2
QRNAPRITFGGPSDS

SEQ ID NO: 372 - protein, 15 aa, SARS-CoV-2
PRITFGGPSDSTGSN

SEQ ID NO: 373 - protein, 15 aa, SARS-CoV-2
FGGPSDSTGSNQNGE

SEQ ID NO: 374 - protein, 15 aa, SARS-CoV-2
SDSTGSNQNGERSGA

SEQ ID NO: 375 - protein, 15 aa, SARS-CoV-2
GSNQNGERSGARSKQ

SEQ ID NO: 376 - protein, 15 aa, SARS-CoV-2
NGERSGARSKQRRPQ

SEQ ID NO: 377 - protein, 15 aa, SARS-CoV-2
SGARSKQRRPQGLPN

SEQ ID NO: 378 - protein, 15 aa, SARS-CoV-2
SKQRRPQGLPNNTAS

SEQ ID NO: 379 - protein, 15 aa, SARS-CoV-2
RPQGLPNNTASWFTA

SEQ ID NO: 380 - protein, 15 aa, SARS-CoV-2
LPNNTASWFTALTQH

SEQ ID NO: 381 - protein, 15 aa, SARS-CoV-2
TASWFTALTQHGKED

SEQ ID NO: 382 - protein, 15 aa, SARS-CoV-2
FTALTQHGKEDLKFP

SEQ ID NO: 383 - protein, 15 aa, SARS-CoV-2
TQHGKEDLKFPRGQG

SEQ ID NO: 384 - protein, 15 aa, SARS-CoV-2
KEDLKFPRGQGVPIN

SEQ ID NO: 385 - protein, 15 aa, SARS-CoV-2
KFPRGQGVPINTNSS

SEQ ID NO: 386 - protein, 15 aa, SARS-CoV-2
GQGVPINTNSSPDDQ

SEQ ID NO: 387 - protein, 15 aa, SARS-CoV-2
PINTNSSPDDQIGYY

SEQ ID NO: 388 - protein, 15 aa, SARS-CoV-2
NSSPDDQIGYYRRAT

SEQ ID NO: 389 - protein, 15 aa, SARS-CoV-2
DDQIGYYRRATRRIR

SEQ ID NO: 390 - protein, 15 aa, SARS-CoV-2
GYYRRATRRIRGGDG

SEQ ID NO: 391 - protein, 15 aa, SARS-CoV-2
RATRRIRGGDGKMKD

SEQ ID NO: 392 - protein, 15 aa, SARS-CoV-2
RIRGGDGKMKDLSPR

SEQ ID NO: 393 - protein, 15 aa, SARS-CoV-2
GDGKMKDLSPRWYFY

SEQ ID NO: 394 - protein, 15 aa, SARS-CoV-2
MKDLSPRWYFYYLGT

SEQ ID NO: 395 - protein, 15 aa, SARS-CoV-2
SPRWYFYYLGTGPEA

SEQ ID NO: 396 - protein, 15 aa, SARS-CoV-2
YFYYLGTGPEAGLPY

SEQ ID NO: 397 - protein, 15 aa, SARS-CoV-2
LGTGPEAGLPYGANK

SEQ ID NO: 398 - protein, 15 aa, SARS-CoV-2
PEAGLPYGANKDGII

SEQ ID NO: 399 - protein, 15 aa, SARS-CoV-2
LPYGANKDGIIWVAT

SEQ ID NO: 400 - protein, 15 aa, SARS-CoV-2
ANKDGIIWVATEGAL

SEQ ID NO: 401 - protein, 15 aa, SARS-CoV-2
GIIWVATEGALNTPK

SEQ ID NO: 402 - protein, 15 aa, SARS-CoV-2
VATEGALNTPKDHIG

SEQ ID NO: 403 - protein, 15 aa, SARS-CoV-2
GALNTPKDHIGTRNP

SEQ ID NO: 404 - protein, 15 aa, SARS-CoV-2
TPKDHIGTRNPANNA

SEQ ID NO: 405 - protein, 15 aa, SARS-CoV-2
HIGTRNPANNAAIVL

SEQ ID NO: 406 - protein, 15 aa, SARS-CoV-2
RNPANNAAIVLQLPQ

SEQ ID NO: 407 - protein, 15 aa, SARS-CoV-2
NNAAIVLQLPQGTTL

SEQ ID NO: 408 - protein, 15 aa, SARS-CoV-2
IVLQLPQGTTLPKGF

SEQ ID NO: 409 - protein, 15 aa, SARS-CoV-2
LPQGTTLPKGFYAEG

SEQ ID NO: 410 - protein, 15 aa, SARS-CoV-2
TTLPKGFYAEGSRGG

SEQ ID NO: 411 - protein, 15 aa, SARS-CoV-2
KGFYAEGSRGGSQAS

SEQ ID NO: 412 - protein, 15 aa, SARS-CoV-2
AEGSRGGSQASSRSS

SEQ ID NO: 413 - protein, 15 aa, SARS-CoV-2
RGGSQASSRSSSRSR

SEQ ID NO: 414 - protein, 15 aa, SARS-CoV-2
QASSRSSSRSRNSSR

SEQ ID NO: 415 - protein, 15 aa, SARS-CoV-2
RSSSRSRNSSRNSTP

SEQ ID NO: 416 - protein, 15 aa, SARS-CoV-2
RSRNSSRNSTPGSSR

SEQ ID NO: 417 - protein, 15 aa, SARS-CoV-2
SSRNSTPGSSRGTSP

SEQ ID NO: 418 - protein, 15 aa, SARS-CoV-2
STPGSSRGTSPARMA

SEQ ID NO: 419 - protein, 15 aa, SARS-CoV-2
SSRGTSPARMAGNGG

SEQ ID NO: 420 - protein, 15 aa, SARS-CoV-2
TSPARMAGNGGDAAL

SEQ ID NO: 421 - protein, 15 aa, SARS-CoV-2
RMAGNGGDAALALLL

SEQ ID NO: 422 - protein, 15 aa, SARS-CoV-2
NGGDAALALLLLDRL

SEQ ID NO: 423 - protein, 15 aa, SARS-CoV-2
AALALLLLDRLNQLE

SEQ ID NO: 424 - protein, 15 aa, SARS-CoV-2
LLLLDRLNQLESKMS

SEQ ID NO: 425 - protein, 15 aa, SARS-CoV-2
DRLNQLESKMSGKGQ

SEQ ID NO: 426 - protein, 15 aa, SARS-CoV-2
QLESKMSGKGQQQQG

SEQ ID NO: 427 - protein, 15 aa, SARS-CoV-2
KMSGKGQQQQGQTVT

SEQ ID NO: 428 - protein, 15 aa, SARS-CoV-2
KGQQQQGQTVTKKSA

SEQ ID NO: 429 - protein, 15 aa, SARS-CoV-2
QQGQTVTKKSAAEAS

SEQ ID NO: 430 - protein, 15 aa, SARS-CoV-2
TVTKKSAAEASKKPR

SEQ ID NO: 431 - protein, 15 aa, SARS-CoV-2
KSAAEASKKPRQKRT

SEQ ID NO: 432 - protein, 15 aa, SARS-CoV-2
EASKKPRQKRTATKA

SEQ ID NO: 433 - protein, 15 aa, SARS-CoV-2
KPRQKRTATKAYNVT

SEQ ID NO: 434 - protein, 15 aa, SARS-CoV-2
KRTATKAYNVTQAFG

SEQ ID NO: 435 - protein, 15 aa, SARS-CoV-2
TKAYNVTQAFGRRGP

SEQ ID NO: 436 - protein, 15 aa, SARS-CoV-2
NVTQAFGRRGPEQTQ

SEQ ID NO: 437 - protein, 15 aa, SARS-CoV-2
AFGRRGPEQTQGNFG

SEQ ID NO: 438 - protein, 15 aa, SARS-CoV-2
RGPEQTQGNFGDQEL

SEQ ID NO: 439 - protein, 15 aa, SARS-CoV-2
QTQGNFGDQELIRQG

SEQ ID NO: 440 - protein, 15 aa, SARS-CoV-2
NFGDQELIRQGTDYK

SEQ ID NO: 441 - protein, 15 aa, SARS-CoV-2
QELIRQGTDYKHWPQ

SEQ ID NO: 442 - protein, 15 aa, SARS-CoV-2
RQGTDYKHWPQIAQF

SEQ ID NO: 443 - protein, 15 aa, SARS-CoV-2
DYKHWPQIAQFAPSA

SEQ ID NO: 444 - protein, 15 aa, SARS-CoV-2
WPQIAQFAPSASAFF

SEQ ID NO: 445 - protein, 15 aa, SARS-CoV-2
AQFAPSASAFFGMSR

SEQ ID NO: 446 - protein, 15 aa, SARS-CoV-2
PSASAFFGMSRIGME

SEQ ID NO: 447 - protein, 15 aa, SARS-CoV-2
AFFGMSRIGMEVTPS

SEQ ID NO: 448 - protein, 15 aa, SARS-CoV-2
MSRIGMEVTPSGTWL

SEQ ID NO: 449 - protein, 15 aa, SARS-CoV-2
GMEVTPSGTWLTYTG

SEQ ID NO: 450 - protein, 15 aa, SARS-CoV-2
TPSGTWLTYTGAIKL

SEQ ID NO: 451 - protein, 15 aa, SARS-CoV-2
TWLTYTGAIKLDDKD

SEQ ID NO: 452 - protein, 15 aa, SARS-CoV-2
YTGAIKLDDKDPNFK

SEQ ID NO: 453 - protein, 15 aa, SARS-CoV-2
IKLDDKDPNFKDQVI

SEQ ID NO: 454 - protein, 15 aa, SARS-CoV-2
DKDPNFKDQVILLNK

SEQ ID NO: 455 - protein, 15 aa, SARS-CoV-2
NFKDQVILLNKHIDA

SEQ ID NO: 456 - protein, 15 aa, SARS-CoV-2
QVILLNKHIDAYKTF

SEQ ID NO: 457 - protein, 15 aa, SARS-CoV-2
LNKHIDAYKTFPPTE

SEQ ID NO: 458 - protein, 15 aa, SARS-CoV-2
IDAYKTFPPTEPKKD

SEQ ID NO: 459 - protein, 15 aa, SARS-CoV-2
KTFPPTEPKKDKKKK

SEQ ID NO: 460 - protein, 15 aa, SARS-CoV-2
PTEPKKDKKKKADET

SEQ ID NO: 461 - protein, 15 aa, SARS-CoV-2
KKDKKKKADETQALP

SEQ ID NO: 462 - protein, 15 aa, SARS-CoV-2
KKKADETQALPQRQK

SEQ ID NO: 463 - protein, 15 aa, SARS-CoV-2
DETQALPQRQKKQQT

SEQ ID NO: 464 - protein, 15 aa, SARS-CoV-2
ALPQRQKKQQTVTLL

SEQ ID NO: 465 - protein, 15 aa, SARS-CoV-2
RQKKQQTVTLLPAAD

SEQ ID NO: 466 - protein, 15 aa, SARS-CoV-2
QQTVTLLPAADLDDF

SEQ ID NO: 467 - protein, 15 aa, SARS-CoV-2
TLLPAADLDDFSKQL

SEQ ID NO: 468 - protein, 15 aa, SARS-CoV-2
AADLDDFSKQLQQSM

SEQ ID NO: 469 - protein, 15 aa, SARS-CoV-2
DDFSKQLQQSMSSAD

SEQ ID NO: 470 - protein, 15 aa, SARS-CoV-2
KQLQQSMSSADSTQA

SEQ ID NO: 471 - protein, 15 aa, SARS-CoV-2
MYSFVSEETGTLIVN

SEQ ID NO: 472 - protein, 15 aa, SARS-CoV-2
VSEETGTLIVNSVLL

SEQ ID NO: 473 - protein, 15 aa, SARS-CoV-2
TGTLIVNSVLLFLAF

SEQ ID NO: 474 - protein, 15 aa, SARS-CoV-2
IVNSVLLFLAFVVFL

SEQ ID NO: 475 - protein, 15 aa, SARS-CoV-2
VLLFLAFVVFLLVTL

SEQ ID NO: 476 - protein, 15 aa, SARS-CoV-2
LAFVVFLLVTLAILT

SEQ ID NO: 477 - protein, 15 aa, SARS-CoV-2
VFLLVTLAILTALRL

SEQ ID NO: 478 - protein, 15 aa, SARS-CoV-2
VTLAILTALRLCAYC

SEQ ID NO: 479 - protein, 15 aa, SARS-CoV-2
ILTALRLCAYCCNIV

SEQ ID NO: 480 - protein, 15 aa, SARS-CoV-2
LRLCAYCCNIVNVSL

SEQ ID NO: 481 - protein, 15 aa, SARS-CoV-2
AYCCNIVNVSLVKPS

SEQ ID NO: 482 - protein, 15 aa, SARS-CoV-2
NIVNVSLVKPSFYVY

SEQ ID NO: 483 - protein, 15 aa, SARS-CoV-2
VSLVKPSFYVYSRVK

SEQ ID NO: 484 - protein, 15 aa, SARS-CoV-2
KPSFYVYSRVKNLNS

SEQ ID NO: 485 - protein, 15 aa, SARS-CoV-2
YVYSRVKNLNSSRVP

SEQ ID NO: 486 - protein, 15 aa, SARS-CoV-2
RVKNLNSSRVPDLLV

SEQ ID NO: 487 - protein, 9 aa, SARS-CoV-2
SPRRARSVA

SEQ ID NO: 488 - protein, 9 aa, SARS-CoV-2
LTDEMIAQY

SEQ ID NO: 489 - protein, 9 aa, SARS-CoV-2
YEQYIKWPW

SEQ ID NO: 490 - protein, 9 aa, SARS-CoV-2
RISNCVADY

SEQ ID NO: 491 - protein, 9 aa, SARS-CoV-2
YNYLYRLFR

SEQ ID NO: 492 - protein, 9 aa, SARS-CoV-2
MTSCCSCLK

SEQ ID NO: 493 - protein, 9 aa, SARS-CoV-2
NSASFSTFK

SEQ ID NO: 494 - protein, 9 aa, SARS-CoV-2
FIAGLIAIV

SEQ ID NO: 495 - protein, 9 aa, SARS-CoV-2
VYSTGSNVF

SEQ ID NO: 496 - protein, 9 aa, SARS-CoV-2
ETKCTLKSF

SEQ ID NO: 497 - protein, 9 aa, SARS-CoV-2
NYNYLYRLF

SEQ ID NO: 498 - protein, 9 aa, SARS-CoV-2
YFPLQSYGF

SEQ ID NO: 499 - protein, 9 aa, SARS-CoV-2
VYYPDKVFR

SEQ ID NO: 500 - protein, 9 aa, SARS-CoV-2
KQGNFKNLR

SEQ ID NO: 501 - protein, 9 aa, SARS-CoV-2
YQDVNCTEV

SEQ ID NO: 502 - protein, 9 aa, SARS-CoV-2
LPFFSNVTW

SEQ ID NO: 503 - protein, 9 aa, SARS-CoV-2
TPGDSSSGW

SEQ ID NO: 504 - protein, 9 aa, SARS-CoV-2
WPWYIWLGF

SEQ ID NO: 505 - protein, 9 aa, SARS-CoV-2
FTISVTTEI

SEQ ID NO: 506 - protein, 9 aa, SARS-CoV-2
NTQEVFAQV

SEQ ID NO: 507 - protein, 9 aa, SARS-CoV-2
KIYSKHTPI

SEQ ID NO: 508 - protein, 9 aa, SARS-CoV-2
FAMQMAYRF

SEQ ID NO: 509 - protein, 9 aa, SARS-CoV-2
TTRTQLPPA

SEQ ID NO: 510 - protein, 9 aa, SARS-CoV-2
ATRFASVYA

SEQ ID NO: 511 - protein, 9 aa, SARS-CoV-2
IAIPTNFTI

SEQ ID NO: 512 - protein, 9 aa, SARS-CoV-2
PYRVVVLSF

SEQ ID NO: 513 - protein, 9 aa, SARS-CoV-2
AENSVAYSN

SEQ ID NO: 514 - protein, 9 aa, SARS-CoV-2
VLNDILSRL

SEQ ID NO: 515 - protein, 9 aa, SARS-CoV-2
GTHWFVTQR

SEQ ID NO: 516 - protein, 9 aa, SARS-CoV-2
KSWMESEFR

SEQ ID NO: 517 - protein, 9 aa, SARS-CoV-2
QIYKTPPIK

SEQ ID NO: 518 - protein, 9 aa, SARS-CoV-2
VLPFNDGVY

SEQ ID NO: 519 - protein, 9 aa, SARS-CoV-2
LAGTITSGW

SEQ ID NO: 520 - protein, 9 aa, SARS-CoV-2
YLQPRTFLL

SEQ ID NO: 521 - protein, 9 aa, SARS-CoV-2
YTNSFTRGV

SEQ ID NO: 522 - protein, 9 aa, SARS-CoV-2
KQIYKTPPI

SEQ ID NO: 523 - protein, 9 aa, SARS-CoV-2
LGAENSVAY

SEQ ID NO: 524 - protein, 9 aa, SARS-CoV-2
ASFSTFKCY

SEQ ID NO: 525 - protein, 9 aa, SARS-CoV-2
SSTASALGK

SEQ ID NO: 526 - protein, 9 aa, SARS-CoV-2
QELGKYEQY

SEQ ID NO: 527 - protein, 9 aa, SARS-CoV-2
IYQTSNFRV

SEQ ID NO: 528 - protein, 9 aa, SARS-CoV-2
FLHVTYVPA

SEQ ID NO: 529 - protein, 9 aa, SARS-CoV-2
SVYAWNRKR

SEQ ID NO: 530 - protein, 9 aa, SARS-CoV-2
NASVVNIQK

SEQ ID NO: 531 - protein, 9 aa, SARS-CoV-2
EVFNATRFA

SEQ ID NO: 532 - protein, 9 aa, SARS-CoV-2
FSTFKCYGV

SEQ ID NO: 533 - protein, 9 aa, SARS-CoV-2
RFDNPVLPF

SEQ ID NO: 534 - protein, 9 aa, SARS-CoV-2
KSFTVEKGI

SEQ ID NO: 535 - protein, 9 aa, SARS-CoV-2
FPQSAPHGV

SEQ ID NO: 536 - protein, 9 aa, SARS-CoV-2
VGGNYNYLY

SEQ ID NO: 537 - protein, 9 aa, SARS-CoV-2
YYVGYLQPR

SEQ ID NO: 538 - protein, 9 aa, SARS-CoV-2
TNSFTRGVY

SEQ ID NO: 539 - protein, 9 aa, SARS-CoV-2
TLADAGFIK

SEQ ID NO: 540 - protein, 9 aa, SARS-CoV-2
VVFLHVTYV

SEQ ID NO: 541 - protein, 9 aa, SARS-CoV-2
LPFNDGVYF

SEQ ID NO: 542 - protein, 9 aa, SARS-CoV-2
NSFTRGVYY

SEQ ID NO: 543 - protein, 9 aa, SARS-CoV-2
LVKQLSSNF

SEQ ID NO: 544 - protein, 9 aa, SARS-CoV-2
ITPCSFGGV

SEQ ID NO: 545 - protein, 9 aa, SARS-CoV-2
KIADYNYKL

SEQ ID NO: 546 - protein, 9 aa, SARS-CoV-2
RARSVASQS

SEQ ID NO: 547 - protein, 9 aa, SARS-CoV-2
LPDDFTGCV

SEQ ID NO: 548 - protein, 9 aa, SARS-CoV-2
PFAMQMAYR

SEQ ID NO: 549 - protein, 9 aa, SARS-CoV-2
ITDAVDCAL

SEQ ID NO: 550 - protein, 9 aa, SARS-CoV-2
GTITSGWTF

SEQ ID NO: 551 - protein, 9 aa, SARS-CoV-2
TLKSFTVEK

SEQ ID NO: 552 - protein, 9 aa, SARS-CoV-2
QTNSPRRAR

SEQ ID NO: 553 - protein, 9 aa, SARS-CoV-2
RQIAPGQTG

SEQ ID NO: 554 - protein, 9 aa, SARS-CoV-2
FVSNGTHWF

SEQ ID NO: 555 - protein, 9 aa, SARS-CoV-2
LPPAYTNSF

SEQ ID NO: 556 - protein, 9 aa, SARS-CoV-2
LPPLLTDEM

SEQ ID NO: 557 - protein, 9 aa, SARS-CoV-2
HLMSFPQSA

SEQ ID NO: 558 - protein, 9 aa, SARS-CoV-2
SKRVDFCGK

SEQ ID NO: 559 - protein, 9 aa, SARS-CoV-2
FQTRAGCLI

SEQ ID NO: 560 - protein, 9 aa, SARS-CoV-2
GWTAGAAAY

SEQ ID NO: 561 - protein, 9 aa, SARS-CoV-2
KCYGVSPTK

SEQ ID NO: 562 - protein, 9 aa, SARS-CoV-2
SVLNDILSR

SEQ ID NO: 563 - protein, 9 aa, SARS-CoV-2
ENGTITDAV

SEQ ID NO: 564 - protein, 9 aa, SARS-CoV-2
YRLFRKSNL

SEQ ID NO: 565 - protein, 9 aa, SARS-CoV-2
IPTNFTISV

SEQ ID NO: 566 - protein, 9 aa, SARS-CoV-2
DVNCTEVPV

SEQ ID NO: 567 - protein, 9 aa, SARS-CoV-2
ITSGWTFGA

SEQ ID NO: 568 - protein, 9 aa, SARS-CoV-2
NATRFASVY

SEQ ID NO: 569 - protein, 9 aa, SARS-CoV-2
LIAIVMVTI

SEQ ID NO: 570 - protein, 9 aa, SARS-CoV-2
STECSNLLL

SEQ ID NO: 571 - protein, 9 aa, SARS-CoV-2
QIAPGQTGK

SEQ ID NO: 572 - protein, 9 aa, SARS-CoV-2
EILPVSMTK

SEQ ID NO: 573 - protein, 9 aa, SARS-CoV-2
GQTGKIADY

SEQ ID NO: 574 - protein, 9 aa, SARS-CoV-2
FPNITNLCP

SEQ ID NO: 575 - protein, 9 aa, SARS-CoV-2
FIKQYGDCL

SEQ ID NO: 576 - protein, 9 aa, SARS-CoV-2
LITGRLQSL

SEQ ID NO: 577 - protein, 9 aa, SARS-CoV-2
TAGAAAYYV

SEQ ID NO: 578 - protein, 9 aa, SARS-CoV-2
YGFQPTNGV

SEQ ID NO: 579 - protein, 9 aa, SARS-CoV-2
KNFTTAPAI

SEQ ID NO: 580 - protein, 9 aa, SARS-CoV-2
FIEDLLFNK

SEQ ID NO: 581 - protein, 9 aa, SARS-CoV-2
VYADSFVIR

SEQ ID NO: 582 - protein, 9 aa, SARS-CoV-2
GVLTESNKK

SEQ ID NO: 583 - protein, 9 aa, SARS-CoV-2
STEKSNIIR

SEQ ID NO: 584 - protein, 9 aa, SARS-CoV-2
YNSASFSTF

SEQ ID NO: 585 - protein, 9 aa, SARS-CoV-2
VLSFELLHA

SEQ ID NO: 586 - protein, 9 aa, SARS-CoV-2
FTNVYADSF

SEQ ID NO: 587 - protein, 9 aa, SARS-CoV-2
DEDDSEPVL

SEQ ID NO: 588 - protein, 9 aa, SARS-CoV-2
DCLGDIAAR

SEQ ID NO: 589 - protein, 9 aa, SARS-CoV-2
LEILDITPC

SEQ ID NO: 590 - protein, 9 aa, SARS-CoV-2
AYSNNSIAI

SEQ ID NO: 591 - protein, 9 aa, SARS-CoV-2
RLDKVEAEV

SEQ ID NO: 592 - protein, 9 aa, SARS-CoV-2
NLCPFGEVF

SEQ ID NO: 593 - protein, 9 aa, SARS-CoV-2
FQPTNGVGY

SEQ ID NO: 594 - protein, 9 aa, SARS-CoV-2
FVSGNCDVV

SEQ ID NO: 595 - protein, 9 aa, SARS-CoV-2
PWYIWLGFI

SEQ ID NO: 596 - protein, 9 aa, SARS-CoV-2
RAAEIRASA

SEQ ID NO: 597 - protein, 9 aa, SARS-CoV-2
KLNDLCFTN

SEQ ID NO: 598 - protein, 9 aa, SARS-CoV-2
ASVYAWNRK

SEQ ID NO: 599 - protein, 9 aa, SARS-CoV-2
LEPLVDLPI

SEQ ID NO: 600 - protein, 9 aa, SARS-CoV-2
SLSSTASAL

SEQ ID NO: 601 - protein, 9 aa, SARS-CoV-2
FPLQSYGFQ

SEQ ID NO: 602 - protein, 9 aa, SARS-CoV-2
NIDGYFKIY

SEQ ID NO: 603 - protein, 9 aa, SARS-CoV-2
QTYVTQQLI

SEQ ID NO: 604 - protein, 15 aa, SARS-CoV-2
GYQPYRVVVLSFELL

SEQ ID NO: 605 - protein, 15 aa, SARS-CoV-2
RVVVLSFELLHAPAT

SEQ ID NO: 606 - protein, 15 aa, SARS-CoV-2
EVFNATRFASVYAWN

SEQ ID NO: 607 - protein, 15 aa, SARS-CoV-2
IGINITRFQTLLALH

SEQ ID NO: 608 - protein, 15 aa, SARS-CoV-2
MFVFLVLLPLVSSQC

SEQ ID NO: 609 - protein, 15 aa, SARS-CoV-2
LHSTQDLFLPFFSNV

SEQ ID NO: 610 - protein, 15 aa, SARS-CoV-2
KRSFIEDLLFNKVTL

SEQ ID NO: 611 - protein, 15 aa, SARS-CoV-2
LFLPFFSNVTWFHAI

SEQ ID NO: 612 - protein, 15 aa, SARS-CoV-2
APHGVVFLHVTYVPA

SEQ ID NO: 613 - protein, 15 aa, SARS-CoV-2
AYYVGYLQPRTFLLK

SEQ ID NO: 614 - protein, 15 aa, SARS-CoV-2
GNFKNLREFVFKNID

SEQ ID NO: 615 - protein, 15 aa, SARS-CoV-2
YLQPRTFLLKYNENG

SEQ ID NO: 616 - protein, 15 aa, SARS-CoV-2
PTNFTISVTTEILPV

SEQ ID NO: 617 - protein, 15 aa, SARS-CoV-2
VFLHVTYVPAQEKNF

SEQ ID NO: 618 - protein, 15 aa, SARS-CoV-2
SFPQSAPHGVVFLHV

SEQ ID NO: 619 - protein, 15 aa, SARS-CoV-2
CTFEYVSQPFLMDLE

SEQ ID NO: 620 - protein, 15 aa, SARS-CoV-2
SVLYNSASFSTFKCY

SEQ ID NO: 621 - protein, 15 aa, SARS-CoV-2
FQFCNDPFLGVYYHK

SEQ ID NO: 622 - protein, 15 aa, SARS-CoV-2
CSNLLLQYGSFCTQL

SEQ ID NO: 623 - protein, 15 aa, SARS-CoV-2
QYIKWPWYIWLGFIA

SEQ ID NO: 624 - protein, 15 aa, SARS-CoV-2
PWYIWLGFIAGLIAI

SEQ ID NO: 625 - protein, 15 aa, SARS-CoV-2
LREFVFKNIDGYFKI

SEQ ID NO: 626 - protein, 15 aa, SARS-CoV-2
YNYLYRLFRKSNLKP

SEQ ID NO: 627 - protein, 15 aa, SARS-CoV-2
IKDFGGFNFSQILPD

SEQ ID NO: 628 - protein, 15 aa, SARS-CoV-2
DLCFTNVYADSFVIR

SEQ ID NO: 629 - protein, 15 aa, SARS-CoV-2
ESNKKFLPFQQFGRD

SEQ ID NO: 630 - protein, 15 aa, SARS-CoV-2
TAGAAAYYVGYLQPR

SEQ ID NO: 631 - protein, 15 aa, SARS-CoV-2
FNCYFPLQSYGFQPT

SEQ ID NO: 632 - protein, 15 aa, SARS-CoV-2
ENQKLIANQFNSAIG

SEQ ID NO: 633 - protein, 15 aa, SARS-CoV-2
DEMIAQYTSALLAGT

SEQ ID NO: 634 - protein, 15 aa, SARS-CoV-2
PSKPSKRSFIEDLLF

SEQ ID NO: 635 - protein, 15 aa, SARS-CoV-2
AGLIAIVMVTIMLCC

SEQ ID NO: 636 - protein, 15 aa, SARS-CoV-2
NIIRGWIFGTTLDSK

SEQ ID NO: 637 - protein, 15 aa, SARS-CoV-2
KVGGNYNYLYRLFRK

SEQ ID NO: 638 - protein, 15 aa, SARS-CoV-2
VYYPDKVFRSSVLHS

SEQ ID NO: 639 - protein, 15 aa, SARS-CoV-2
GTGVLTESNKKFLPF

SEQ ID NO: 640 - protein, 15 aa, SARS-CoV-2
NDGVYFASTEKSNII

SEQ ID NO: 641 - protein, 15 aa, SARS-CoV-2
TRFQTLLALHRSYLT

SEQ ID NO: 642 - protein, 15 aa, SARS-CoV-2
RLFRKSNLKPFERDI

SEQ ID NO: 643 - protein, 15 aa, SARS-CoV-2
LDSFKEELDKYFKNH

SEQ ID NO: 644 - protein, 15 aa, SARS-CoV-2
LQSLQTYVTQQLIRA

SEQ ID NO: 645 - protein, 15 aa, SARS-CoV-2
FGAISSVLNDILSRL

SEQ ID NO: 646 - protein, 15 aa, SARS-CoV-2
QKFNGLTVLPPLLTD

SEQ ID NO: 647 - protein, 15 aa, SARS-CoV-2
FVTQRNFYEPQIITT

SEQ ID NO: 648 - protein, 15 aa, SARS-CoV-2
IKVCEFQFCNDPFLG

SEQ ID NO: 649 - protein, 15 aa, SARS-CoV-2
EHVNNSYECDIPIGA

SEQ ID NO: 650 - protein, 15 aa, SARS-CoV-2
CNGVEGFNCYFPLQS

SEQ ID NO: 651 - protein, 15 aa, SARS-CoV-2
DPLQPELDSFKEELD

SEQ ID NO: 652 - protein, 15 aa, SARS-CoV-2
AAEIRASANLAATKM

SEQ ID NO: 653 - protein, 15 aa, SARS-CoV-2
SLLIVNNATNVVIKV

SEQ ID NO: 654 - protein, 15 aa, SARS-CoV-2
TQLNRALTGIAVEQD

SEQ ID NO: 655 - protein, 15 aa, SARS-CoV-2
TNTSNQVAVLYQDVN

SEQ ID NO: 656 - protein, 15 aa, SARS-CoV-2
ASANLAATKMSECVL

SEQ ID NO: 657 - protein, 15 aa, SARS-CoV-2
FGAGAALQIPFAMQM

SEQ ID NO: 658 - protein, 15 aa, SARS-CoV-2
QYTSALLAGTITSGW

SEQ ID NO: 659 - protein, 15 aa, SARS-CoV-2
TYVTQQLIRAAEIRA

SEQ ID NO: 660 - protein, 15 aa, SARS-CoV-2
TWRVYSTGSNVFQTR

SEQ ID NO: 661 - protein, 15 aa, SARS-CoV-2
GDISGINASVVNIQK

SEQ ID NO: 662 - protein, 15 aa, SARS-CoV-2
FNFNGLTGTGVLTES

SEQ ID NO: 663 - protein, 15 aa, SARS-CoV-2
EDLLFNKVTLADAGF

SEQ ID NO: 664 - protein, 15 aa, SARS-CoV-2
DSSSGWTAGAAAYYV

SEQ ID NO: 665 - protein, 15 aa, SARS-CoV-2
VVNQNAQALNTLVKQ

SEQ ID NO: 666 - protein, 15 aa, SARS-CoV-2
AKNLNESLIDLQELG

SEQ ID NO: 667 - protein, 15 aa, SARS-CoV-2
LDKVEAEVQIDRLIT

SEQ ID NO: 668 - protein, 15 aa, SARS-CoV-2
ITSGWTFGAGAALQI

SEQ ID NO: 669 - protein, 15 aa, SARS-CoV-2
DLPQGFSALEPLVDL

SEQ ID NO: 670 - protein, 15 aa, SARS-CoV-2
ALTGIAVEQDKNTQE

SEQ ID NO: 671 - protein, 15 aa, SARS-CoV-2
INASVVNIQKEIDRL

SEQ ID NO: 672 - protein, 15 aa, SARS-CoV-2
NCTEVPVAIHADQLT

SEQ ID NO: 673 - protein, 15 aa, SARS-CoV-2
NVYADSFVIRGDEVR

SEQ ID NO: 674 - protein, 15 aa, SARS-CoV-2
PVAIHADQLTPTWRV

SEQ ID NO: 675 - protein, 15 aa, SARS-CoV-2
DIPIGAGICASYQTQ

SEQ ID NO: 676 - protein, 15 aa, SARS-CoV-2
LDITPCSFGGVSVIT

SEQ ID NO: 677 - protein, 15 aa, SARS-CoV-2
CSFGGVSVITPGTNT

SEQ ID NO: 678 - protein, 15 aa, SARS-CoV-2
VKQLSSNFGAISSVL

SEQ ID NO: 679 - protein, 15 aa, SARS-CoV-2
NPVLPFNDGVYFAST

SEQ ID NO: 680 - protein, 15 aa, SARS-CoV-2
SFELLHAPATVCGPK

SEQ ID NO: 681 - protein, 15 aa, SARS-CoV-2
QIPFAMQMAYRFNGI

SEQ ID NO: 682 - protein, 15 aa, SARS-CoV-2
LTVLPPLLTDEMIAQ

SEQ ID NO: 683 - protein, 15 aa, SARS-CoV-2
AEVQIDRLITGRLQS

SEQ ID NO: 684 - protein, 15 aa, SARS-CoV-2
DGYFKIYSKHTPINL

SEQ ID NO: 685 - protein, 15 aa, SARS-CoV-2
INLVRDLPQGFSALE

SEQ ID NO: 686 - protein, 15 aa, SARS-CoV-2
SFVIRGDEVRQIAPG

SEQ ID NO: 687 - protein, 15 aa, SARS-CoV-2
ISNCVADYSVLYNSA

SEQ ID NO: 688 - protein, 15 aa, SARS-CoV-2
FYEPQIITTDNTFVS

SEQ ID NO: 689 - protein, 15 aa, SARS-CoV-2
IITTDNTFVSGNCDV

SEQ ID NO: 690 - protein, 15 aa, SARS-CoV-2
KVFRSSVLHSTQDLF

SEQ ID NO: 691 - protein, 15 aa, SARS-CoV-2
APAICHDGKAHFPRE

SEQ ID NO: 692 - protein, 15 aa, SARS-CoV-2
SFTRGVYYPDKVFRS

SEQ ID NO: 693 - protein, 15 aa, SARS-CoV-2
SVLNDILSRLDKVEA

SEQ ID NO: 694 - protein, 15 aa, SARS-CoV-2
GVTQNVLYENQKLIA

SEQ ID NO: 695 - protein, 15 aa, SARS-CoV-2
VSQPFLMDLEGKQGN

SEQ ID NO: 696 - protein, 15 aa, SARS-CoV-2
GFNFSQILPDPSKPS

SEQ ID NO: 697 - protein, 15 aa, SARS-CoV-2
LQYGSFCTQLNRALT

SEQ ID NO: 698 - protein, 15 aa, SARS-CoV-2
QTSNFRVQPTESIVR

SEQ ID NO: 699 - protein, 15 aa, SARS-CoV-2
DPFLGVYYHKNNKSW

SEQ ID NO: 700 - protein, 15 aa, SARS-CoV-2
EGVFVSNGTHWFVTQ

SEQ ID NO: 701 - protein, 15 aa, SARS-CoV-2
IQDSLSSTASALGKL

SEQ ID NO: 702 - protein, 15 aa, SARS-CoV-2
WFHAIHVSGTNGTKR

SEQ ID NO: 703 - protein, 15 aa, SARS-CoV-2
AGICASYQTQTNSPR

SEQ ID NO: 704 - protein, 15 aa, SARS-CoV-2
GNCDVVIGIVNNTVY

SEQ ID NO: 705 - protein, 15 aa, SARS-CoV-2
KPFERDISTEIYQAG

SEQ ID NO: 706 - protein, 15 aa, SARS-CoV-2
QPTESIVRFPNITNL

SEQ ID NO: 707 - protein, 15 aa, SARS-CoV-2
NGTHWFVTQRNFYEP

SEQ ID NO: 708 - protein, 15 aa, SARS-CoV-2
MQMAYRFNGIGVTQN

SEQ ID NO: 709 - protein, 15 aa, SARS-CoV-2
RFNGIGVTQNVLYEN

SEQ ID NO: 710 - protein, 15 aa, SARS-CoV-2
EELDKYFKNHTSPDV

SEQ ID NO: 711 - protein, 15 aa, SARS-CoV-2
SWMESEFRVYSSANN

SEQ ID NO: 712 - protein, 15 aa, SARS-CoV-2
FSNVTWFHAIHVSGT

SEQ ID NO: 713 - protein, 15 aa, SARS-CoV-2
GTTLDSKTQSLLIVN

SEQ ID NO: 714 - protein, 15 aa, SARS-CoV-2
PRRARSVASQSIIAY

SEQ ID NO: 715 - protein, 15 aa, SARS-CoV-2
STGSNVFQTRAGCLI

SEQ ID NO: 716 - protein, 15 aa, SARS-CoV-2
LLALHRSYLTPGDSS

SEQ ID NO: 717 - protein, 15 aa, SARS-CoV-2
AQALNTLVKQLSSNF

SEQ ID NO: 718 - protein, 15 aa, SARS-CoV-2
SQSIIAYTMSLGAEN

SEQ ID NO: 719 - protein, 15 aa, SARS-CoV-2
FRVYSSANNCTFEYV

SEQ ID NO: 720 - protein, 15 aa, SARS-CoV-2
TRFASVYAWNRKRIS

SEQ ID NO: 721 - protein, 15 aa, SARS-CoV-2
VYAWNRKRISNCVAD

SEQ ID NO: 722 - protein, 15 aa, SARS-CoV-2
CGKGYHLMSFPQSAP

SEQ ID NO: 723 - protein, 15 aa, SARS-CoV-2
DDSEPVLKGVKLHYT

SEQ ID NO: 724 - protein, 15 aa, SARS-CoV-2
DRLITGRLQSLQTYV

SEQ ID NO: 725 - protein, 15 aa, SARS-CoV-2
TFLLKYNENGTITDA

SEQ ID NO: 726 - protein, 15 aa, SARS-CoV-2
GKLQDVVNQNAQALN

SEQ ID NO: 727 - protein, 15 aa, SARS-CoV-2
AENSVAYSNNSIAIP

SEQ ID NO: 728 - protein, 15 aa, SARS-CoV-2
RLNEVAKNLNESLID

SEQ ID NO: 729 - protein, 15 aa, SARS-CoV-2
STNLVKNKCVNFNFN

SEQ ID NO: 730 - protein, 15 aa, SARS-CoV-2
CVIAWNSNNLDSKVG

SEQ ID NO: 731 - protein, 15 aa, SARS-CoV-2
LVDLPIGINITRFQT

SEQ ID NO: 732 - protein, 15 aa, SARS-CoV-2
SMTKTSVDCTMYICG

SEQ ID NO: 733 - protein, 15 aa, SARS-CoV-2
QFGRDIADTTDAVRD

SEQ ID NO: 734 - protein, 15 aa, SARS-CoV-2
EKGIYQTSNFRVQPT

SEQ ID NO: 735 - protein, 15 aa, SARS-CoV-2
AYTMSLGAENSVAYS

SEQ ID NO: 736 - protein, 15 aa, SARS-CoV-2
KNKCVNFNFNGLTGT

SEQ ID NO: 737 - protein, 15 aa, SARS-CoV-2
LLPLVSSQCVNLTTR

SEQ ID NO: 738 - protein, 15 aa, SARS-CoV-2
EVFAQVKQIYKTPPI

SEQ ID NO: 739 - protein, 15 aa, SARS-CoV-2
KNFTTAPAICHDGKA

SEQ ID NO: 740 - protein, 15 aa, SARS-CoV-2
LCPFGEVFNATRFAS

SEQ ID NO: 741 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 742 - protein, 222 aa, SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 743 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 744 - protein, 75 aa, SARS-CoV-2
MYSFVSEETGTLIVNSVLLFLAFVVFLLVTLAILTALRLCAYCCNIVNVSLVKPSFYVYSRVKNLNSSRVPDLLV